SEQUENCE LISTING (28 sequences)

SEQ ID NO: 1 - DNA, 100 bp, Homo sapiens
  source 1..100: mol_type = genomic DNA
actaccagacacaacagcagaaactctttcctcagctggccatcagttatgccttccatttcctggcagtcagcctcttggagttcttccagcactccta

SEQ ID NO: 2 - DNA, 100 bp, Homo sapiens
  source 1..100: mol_type = genomic DNA
ccagggaaggttcatatcacaaagacgaccctagcgtgcttgaatggggactacgaggtagaaccgggttacggacatgagaggaacagtttcttgaaaa

SEQ ID NO: 3 - DNA, 100 bp, Homo sapiens
  source 1..100: mol_type = genomic DNA
tactgaagaatggagagagaattgaaaaagtggagcattcagacttgtctttcagcaaggactggtctttctatctcttgtactacactgaattcacccc

SEQ ID NO: 4 - DNA, 100 bp, Homo sapiens
  source 1..100: mol_type = genomic DNA
ttacatctgccatcaggagctgaggaatgaaccagccaacaaccaaggcgaggagagtgctgagatcatccctttgaatatcatagagcaagagtcatct

SEQ ID NO: 5 - DNA, 100 bp, Homo sapiens
  source 1..100: mol_type = genomic DNA
agtttgagcaccccaacagctactatcacccggtcttcggcaaggccatcctggcccggtaccgcgccaatgcctctcgggaggccctgcggaccggctc

SEQ ID NO: 6 - DNA, 100 bp, Homo sapiens
  source 1..100: mol_type = genomic DNA
ttggcacaacaggaagctgttgaaggaggatgttcccatcttggtcagtcctatgcggatagagatgtctggaagccagaaccatgccaaatatgtgtct

SEQ ID NO: 7 - DNA, 100 bp, Homo sapiens
  source 1..100: mol_type = genomic DNA
ccgcccgcccgcccgcccgcgccatgaacgccaaggtcgtggtcgtgctggtcctcgtgctgaccgcgctctgcctcagcgacgggaagcccgtcagcct

SEQ ID NO: 8 - DNA, 100 bp, Homo sapiens
  source 1..100: mol_type = genomic DNA
gtaacaagtatatgaagaacagacctgctctttctctcaggggtatcctcaccttgtataatcttggaatcacacttctctccgcgtacatgctggcaga

SEQ ID NO: 9 - DNA, 100 bp, Homo sapiens
  source 1..100: mol_type = genomic DNA
gaacgggaagcttgtcatcaatggaaatcccatcaccatcttccaggagcgagatccctccaaaatcaagtggggcgatgctggcgctgagtacgtcgtg

SEQ ID NO: 10 - DNA, 100 bp, Homo sapiens
  source 1..100: mol_type = genomic DNA
ccagcagggtctgagaatgaaattcccactcatgttttgccaccgtgtgcaaatttacaggcacagaagctgaaatccaatgtgtcgggcaatacacacc

SEQ ID NO: 11 - DNA, 100 bp, Homo sapiens
  source 1..100: mol_type = genomic DNA
gcgaacctacatcatcagtatcctcttcaagtctatctttgaggtggccttcttgctgatccagtggtacatctatggattcagcttgagtgctgtttac

SEQ ID NO: 12 - DNA, 100 bp, Homo sapiens
  source 1..100: mol_type = genomic DNA
cagcttcagtcacctttccagtggtggcctctggagaaccagtgtctgttcctgacaacttgctgaaaatatgcaaggccaagccagtgatatttaaagg

SEQ ID NO: 13 - DNA, 100 bp, Homo sapiens
  source 1..100: mol_type = genomic DNA
tctcctgatggtgggccctctgtgctcttctcttccggtcggatctctctcctctctgacctggatacgctttggtttctcaacttctctaccccaaaga

SEQ ID NO: 14 - DNA, 100 bp, Homo sapiens
  source 1..100: mol_type = genomic DNA
actgccaacagtttcggctgcatttcttcacgcacctcggttcctcttcctgaagttcttgtgccctgctcttcagcaccatgggccttcttatacggaa

SEQ ID NO: 15 - DNA, 100 bp, Homo sapiens
  source 1..100: mol_type = genomic DNA
tgaaaccagaggactttgacaatgttcagtccctcctgacaaacagtatttacttacaagattcggaggtaacagtgaagggattcaggatatacggtgc

SEQ ID NO: 16 - DNA, 100 bp, Homo sapiens
  source 1..100: mol_type = genomic DNA
ccgcgcccgccgcgccatggcccgaagaccccggcacagcatatatagcagtgacgaggatgatgaggactttgagatgtgtgaccatgactatgatggg

SEQ ID NO: 17 - DNA, 100 bp, Homo sapiens
  source 1..100: mol_type = genomic DNA
acatacttctcagctgcaaatattatggagaattggggcacccacaggaatgaagagagaaagcagctccctaacttcaaaaccattttggtacctgaca

SEQ ID NO: 18 - DNA, 100 bp, Homo sapiens
  source 1..100: mol_type = genomic DNA
cacagcagaccatgtgttcacgggatgcccgcacaaaacagctgaggcagctactggagaaggtgcagaacatgtctcaatccatagaggtcttggacag

SEQ ID NO: 19 - DNA, 100 bp, Homo sapiens
  source 1..100: mol_type = genomic DNA
accgagggccacctggtccgggtggttgagaagtgtagcccagcagagaaggctggccttcaagatggagacagagttcttaggatcaatggtgtctttg

SEQ ID NO: 20 - DNA, 100 bp, Homo sapiens
  source 1..100: mol_type = genomic DNA
agccagccagagcccacaatacagcttcgagtcattacctcagaagatttgtttaatctgtggggatgaagcatcaggctgtcattatggtgtccttacc

SEQ ID NO: 21 - DNA, 100 bp, Homo sapiens
  source 1..100: mol_type = genomic DNA
catcggacaatttctggaggctgtggatcagaatacagccatcgtgggctctaccacaggctccaactattatgtgcgcatcctgagcaccatcgatcgg

SEQ ID NO: 22 - DNA, 100 bp, Homo sapiens
  source 1..100: mol_type = genomic DNA
ctggggaacatcagatcattcagtttcccagccaatcatggtgcagagaagacctggtcagagtttccatgtgaacagtgaggtcaattctgtactgtcc

SEQ ID NO: 23 - DNA, 100 bp, Homo sapiens
  source 1..100: mol_type = genomic DNA
acgatctcattgacagctgcattcaatcttttgatgcagatggaaacctgtgtcgaagtaaccaactgttgcaagtcatgctgaccatgcaccgaattgt

SEQ ID NO: 24 - DNA, 100 bp, Homo sapiens
  source 1..100: mol_type = genomic DNA
gtgtgaaccgtcgtgtgagtgtggtatgcctgatcacagatggattttgttataagcatcaatgtgacacttgcaggacactacaacgtgggacattgtt

SEQ ID NO: 25 - DNA, 100 bp, Homo sapiens
  source 1..100: mol_type = genomic DNA
atttctatgtgtaatttctgagccattgtactgtctgggctgggggggacactgtccaagggagtggcccctatgagtttatattttaaccactgcttca

SEQ ID NO: 26 - DNA, 100 bp, Homo sapiens
  source 1..100: mol_type = genomic DNA
tttcgacgacaccgttcgtggggtcccctggtgcttctatcctaataccatcgacgtccctccagaagaggagtgtgaattttagacacttctgcaggga

SEQ ID NO: 27 - DNA, 100 bp, Homo sapiens
  source 1..100: mol_type = genomic DNA
agggctttatctaccagggctcggccaagttcatcaagaacataccttggaattttgggcagtttgaagatgggatcctagacatctgcctgatccttct

SEQ ID NO: 28 - DNA, 100 bp, Homo sapiens
  source 1..100: mol_type = genomic DNA
gcagggtgcccatctcacaagcatcctgtctcacgaagaacaaatgtttgttaatcgtgtgggccatgattatcagtggataggcctcaatgacaagatg

(19) United States
(12) Patent Application Publication
(10) Pub. No.: US 2025/0154597 A1
(43) Pub. Date: May 15, 2025

(54) TRANSCRIPTIONAL REPROGRAMMING DIFFERENTIATES ACTIVE FROM INACTIVE ESR1 FUSIONS IN ENDOCRINE THERAPY-REFRACTORY METASTATIC BREAST CANCER

(71) Applicant: Baylor College of Medicine, Houston, TX (US)

(72) Inventors: Matthew J. Ellis, Houston, TX (US); Xuxu Gou, Houston, TX (US); Meenakshi Anurag, Cypress, TX (US); Jonathan T. Lei, Houston, TX (US); Charles E. Foulds, Houston, TX (US)

(73) Assignee: Baylor College of Medicine, Houston, TX (US)

(21) Appl. No.: 18/700,510

(22) PCT Filed: Oct. 11, 2022
(86) PCT No.: PCT/US2022/077924

§ 371 Date: Apr. 11, 2024

Related U.S. Application Data
(60) Provisional application No. 63/254,890, filed on Oct. 12, 2021.

(51) Int. Cl. C12Q 1/6886; C12Q 1/6874

(52) U.S. Cl. CPC C12Q 1/6886; C12Q 1/6874; C12Q 2600/156; C12Q 2600/158

(57) ABSTRACT

Methods of treatment, methods of detection, and kits associated with ERα+ cancer, such as ERα+ breast cancer, are disclosed herein. The methods and kits disclosed herein can assist physicians in relieving patient suffering by identifying cancer estrogen receptor status, and identifying appropriate therapeutic regimens in an individualized manner.

### CROSS-REFERENCE TO RELATED APPLICATIONS

This application claims priority to U.S. Provisional Patent Application Ser. No. 63/254,890, filed Oct. 12, 2021, which is incorporated by reference herein in its entirety.

### STATEMENT REGARDING FEDERALLY SPONSORED RESEARCH OR DEVELOPMENT

This invention was made with government support under CA186784 and CA233223 awarded by the National Institutes of Health, and W81XWH2110119 awarded by the Department of Defense. The government has certain rights in the invention.

### BACKGROUND

This disclosure relates to the field of cancer biology, genetics, medicine, and therapeutic treatment methods.

Despite recent advances, the challenge of cancer treatment remains to target specific treatment regimens to distinct tumor types with different pathogenesis, and ultimately personalize tumor treatment in order to maximize outcome. In particular, once a patient is diagnosed with cancer, such as breast cancer, there is a need for methods that allow the physician to predict the expected course of disease, including the likelihood of cancer recurrence, long-term survival of the patient and the like, and select the most appropriate treatment options accordingly. There exists a need for new and improved methods for diagnosis, prognosis, and informed treatment of cancer, including breast cancer, based on analysis of tumor mutations, gene expression, and epigenetics.

### SUMMARY

Methods for classifying and for evaluating prognosis and treatment of a subject with cancer are provided herein. Compositions and kits suitable for evaluating prognosis and treatment of a subject with cancer are also provided herein. Also provided herein are methods, compositions, and kits suitable for determining mutation and/or translocation status of the endogenous ESR1 locus in a patient.

In some embodiments, disclosed is a method of treating cancer, comprising: administering a non-endocrine therapy (ET) resistant therapeutic regimen to a patient after identification of ESR1 protein activity, wherein ESR1 protein activity is determined by measuring a biological sample from the patient for estrogen response gene expression, protein levels and/or protein activity, and/or epithelial to mesenchymal transition (EMT) gene expression, protein levels, and/or protein activity. In some embodiments, gene expression measuring comprises measuring of messenger RNA (mRNA) levels. In some embodiments, gene expression measuring comprises measuring of transcribed RNA (e.g., pre-mRNA and/or mRNA) levels. In some embodiments, gene expression measuring comprises measuring of translated protein product levels and/or activity.

In some embodiments, estrogen response gene expression and/or EMT gene expression is determined by measuring the level of expression for at least six genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, estrogen response gene expression and/or EMT gene expression is determined by measuring the level of expression for at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, estrogen response gene expression and/or EMT gene expression is determined by measuring the level of expression for at least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, estrogen response gene expression and EMT gene expression is determined by measuring the level of expression for at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, determination of estrogen response gene expression comprises measuring the level of expression of genes: ADCY1, GREB1, MYB, NPY1R, PGR, and TFF1. In some embodiments, determination of estrogen response gene expression and EMT gene expression comprises measuring the level of expression of genes: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, determination of estrogen response gene expression further comprises measuring expression levels of one or more internal controls. In some embodiments, one or more internal controls comprise B2M, GAPDH, PSMC4, and/or PUM1.

In some embodiments, the level of gene expression is increased relative to a control and is identified using a nucleotide quantification assay. In some embodiments a nucleotide quantification assay is a gene chip assay or RNA sequencing. In some embodiments, the nucleotide quantification assay comprises a labeled probe-based hybridization analysis assay. In some embodiments, the assay comprises one or more targeting probe/primers which comprises or consists of, or comprises or consists of a sequence complementary to SEQ ID NO: 1 to SEQ ID NO: 28. In some embodiments, the label comprises a chromogenic label, fluorescent label, epitope label, and/or hapten label. In some embodiments, the labeled probe-based hybridization analysis assay comprises a NanoString assay. In some embodiments, the measuring the level of expression comprises measuring expression of mRNA levels.

In some embodiments, the cancer is breast cancer. In some embodiments, the breast cancer is ERα+ metastatic breast cancer (MBC).

In some embodiments, a non-endocrine therapy (non-ET) resistant therapeutic regimen comprises a cyclin-dependent kinase 4/6 (CDK4/6) inhibitor. In some embodiments, a non-ET resistant therapeutic regimen comprises a CDK4/6 inhibitor, and one or more of a selective ER modulator (SERM), an aromatase inhibitor, and/or a selective ER down-regulator (SERD). In some embodiments, a CDK4/6 inhibitor is abemaciclib, palbociclib, or ribociclib.

In some embodiments, a non-ET resistant therapeutic regimen comprises at least one chemotherapeutic agent, wherein the chemotherapeutic agent is at least one of capecitabine, carboplatin, cyclophosphamide, daunorubicin, docetaxel, doxorubicin, epirubicin, fluorouracil, gemcitabine, eribulin, ixabepilone, methotrexate, mitomycin C, mitoxantrone, paclitaxel, thiotepa, vincristine, or vinorelbin.

In some embodiments, a reflexive diagnostic test is performed following ESR1 protein activity determination and prior to treatment regimen initiation. In some embodiments, a reflexive diagnostic test is selected from unbiased RNA-Seq, whole exome sequencing, ESR1-specific 3′ Rapid Amplification of cDNA ends (3′-RACE), and break-apart ESR1 fluorescence in situ hybridization (FISH).

In some embodiments, at least a portion of the endogenous ESR1 locus sequence is determined. In some embodiments, the endogenous ESR1 locus is determined to have a 3′ fusion (e.g., a 3′ fusion to a partner gene). In some embodiments, the endogenous ESR1 locus is determined to have a point mutation.

In some embodiments, ESR1 protein activity confers tumor cell resistance to non-combinatorial endocrine therapy (ET)s. In some embodiments, administration of a non-ET resistant therapeutic regimen occurs within 1 month after identification of ESR1 protein activity.

In some embodiments, a biological sample is a primary tumor tissue sample. In some embodiments, a biological sample is a metastatic tumor lesion sample.

Also disclosed herein is a method of treating metastatic breast cancer in a patient, comprising identification of ESR1 protein activity, and administration of a cancer therapy comprising at least one of chemotherapy, cryoablative therapy, hi-intensity ultrasound, photodynamic therapy, laser ablation, irreversible electroporation, ET, radiotherapy, surgery, immunotherapy, or a combination thereof, wherein, ESR1 protein activity is determined by measuring expression levels of at least six genes selected from ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B in a patient's biological sample.

In some embodiments, ESR1 protein activity is determined by measuring the level of expression for at least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ESR1 protein activity is determined by measuring the level of expression for at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ESR1 protein activity is determined by measuring the level of expression for at least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ESR1 protein activity is determined by measuring the level of expression for at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

In some embodiments, determination of ESR1 protein activity comprises measuring the level of expression of genes: ADCY1, GREB1, MYB, NPY1R, PGR, and TFF1. In some embodiments, determination of ESR1 protein activity comprises measuring the level of expression of genes: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, determination of ESR1 protein activity further comprises measuring expression levels of one or more internal controls. In some embodiments, one or more internal controls comprise B2M, GAPDH, PSMC4, and/or PUM1.

In some embodiments, a level of gene expression is increased relative to a control and is identified using a nucleotide quantification assay. In some embodiments, a nucleotide quantification assay is a gene chip assay or RNA sequencing. In some embodiments, the nucleotide quantification assay comprises a labeled probe-based hybridization analysis assay. In some embodiments, the assay comprises one or more targeting probe/primers which comprises or consists of, or comprises or consists of a sequence complementary to SEQ ID NO: 1 to SEQ ID NO: 28. In some embodiments, the labeled probe-based hybridization analysis assay comprises a NanoString assay. In some embodiments, the measuring the level of expression comprises measuring expression of mRNA levels.

In some embodiments, a metastatic breast cancer is ERα+.

In some embodiments, an anti-cancer therapeutic regimen comprises a CDK4/6 inhibitor. In some embodiments, an anti-cancer therapeutic regimen comprises a CDK4/6 inhibitor, and one or more of a SERM, an aromatase inhibitor, and/or a SERD. In some embodiments, a CDK4/6 inhibitor is abemaciclib, palbociclib, or ribociclib.

In some embodiments, an anti-cancer therapeutic regimen comprises at least one chemotherapeutic agent, wherein the chemotherapeutic agent is at least one of capecitabine, carboplatin, cyclophosphamide, daunorubicin, docetaxel, doxorubicin, epirubicin, fluorouracil, gemcitabine, eribulin, ixabepilone, methotrexate, mitomycin C, mitoxantrone, paclitaxel, thiotepa, vincristine, or vinorelbin. In some embodiments, for ER+ cancer (e.g., ER+ metastatic breast cancer), an anti-cancer therapeutic regimen comprises capecitabine.

In some embodiments, administering of an anti-cancer therapeutic regimen occurs within 1 month after identification of ESR1 protein activity.

In some embodiments, disclosed is a method of treating metastatic breast cancer comprising, administering an effective amount of a non-ET resistant therapeutic regimen to a patient determined to have a biological sample with increased tumor cell expression of at least six genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a patient is determined to have a biological sample with increased tumor cell expression of at least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a patient is determined to have a biological sample with increased tumor cell expression of at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a patient is determined to have a biological sample with increased tumor cell expression of at least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a patient is determined to have a biological sample with increased tumor cell expression of at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

In some embodiments, a patient is determined to have a biological sample with increased tumor cell expression of genes: ADCY1, GREB1, MYB, NPY1R, PGR, and TFF1. In some embodiments, a patient is determined to have a biological sample with increased tumor cell expression of: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, determination of increased tumor cell expression further comprises measuring expression levels of one or more internal controls. In some embodiments, one or more internal controls comprise B2M, GAPDH, PSMC4, and/or PUM1.

In some embodiments, a level of gene expression is increased relative to a control and is identified using a nucleotide quantification assay. In some embodiments, a nucleotide quantification assay is a gene chip assay or RNA sequencing. In some embodiments, the nucleotide quantification assay comprises a labeled probe-based hybridization analysis assay. In some embodiments, the assay comprises one or more targeting probe/primers which comprises or consists of, or comprises or consists of a sequence complementary to SEQ ID NO: 1 to SEQ ID NO: 28. In some embodiments, the labeled probe-based hybridization analysis assay comprises a NanoString assay. In some embodiments, the measuring the level of expression comprises measuring expression of mRNA levels.

Also disclosed herein is a method for treating a subject for breast cancer, the method comprising: (a) detecting cancer sample gene expression levels for at least six genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B; and (b) administering an effective amount of a breast cancer therapy to the subject.

In some embodiments, a subject is determined to have a cancer sample with increased expression of at least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a subject is determined to have a cancer sample with increased expression of at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a subject is determined to have a cancer sample with increased expression of least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a subject is determined to have a cancer sample with increased expression of at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

In some embodiments, a subject is determined to have a cancer sample with increased expression of at least genes: ADCY1, GREB1, MYB, NPY1R, PGR, and TFF1. In some embodiments, the subject is determined to have a cancer sample with increased expression of at least genes: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, the method further comprises measuring expression levels of one or more internal controls. In some embodiments, the one or more internal controls comprise B2M, GAPDH, PSMC4, and/or PUM1.

In some embodiments, a subject is determined to have a cancer sample wherein the level of target gene activity is increased relative to a control and is identified using a nucleotide quantification assay. In some embodiments, the nucleotide quantification assay is a gene chip assay or RNA sequencing. In some embodiments, the nucleotide quantification assay comprises a labeled probe-based hybridization analysis assay. In some embodiments, the assay comprises one or more targeting probe/primers which comprises or consists of, or comprises or consists of a sequence complementary to SEQ ID NO: 1 to SEQ ID NO: 28. In some embodiments, the labeled probe-based hybridization analysis assay comprises a NanoString assay. In some embodiments, the measuring of gene activity comprises measuring expression of mRNA levels.

In some embodiments, increased expression of cancer sample genes is representative of tumor cell resistance to non-combinatorial ETs.

Also disclosed herein is a method for treating cancer in a patient, the method comprising administering a cancer therapy comprising an effective amount of a CDK4/6 inhibitor to the patient after determining whether the patient has a mutant or translocated estrogen receptor alpha (ERα) protein, wherein ERα protein activity is determined by measuring a biological sample from the patient for estrogen response gene expression, protein levels, and/or protein activity, and/or epithelial to mesenchymal transition (EMT) gene expression, protein levels, and/or protein activity In some embodiments, gene expression measuring comprises measuring of messenger RNA (mRNA) levels. In some embodiments, gene expression measuring comprises measuring of pre-mRNA and/or mRNA levels. In some embodiments, gene expression measuring comprises measuring of translated protein product levels and/or activity.

In some embodiments, estrogen response gene expression and/or EMT gene expression is determined by measuring expression levels of least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, estrogen response gene expression and/or EMT gene expression is determined by measuring expression levels of least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, estrogen response gene expression and/or EMT gene expression is determined by measuring expression levels of least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, estrogen response gene expression and/or EMT gene expression is determined by measuring expression levels of least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

In some embodiments, determination of estrogen response gene expression comprises measuring of expression levels of genes: ADCY1, GREB1, MYB, NPY1R, PGR, and TFF1. In some embodiments, determination of estrogen response gene expression comprises measuring of expression levels of genes: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, the method further comprises measuring expression levels of one or more internal controls. In some embodiments, the one or more internal controls comprise B2M, GAPDH, PSMC4, and/or PUM1.

In some embodiments, determination of estrogen response gene expression comprises measuring of expression levels of target genes relative to a control, and is determined using a nucleotide quantification assay. In some embodiments, the nucleotide quantification assay is a gene chip assay or RNA sequencing. In some embodiments, the nucleotide quantification assay comprises a labeled probe-based hybridization analysis assay. In some embodiments, the assay comprises one or more targeting probe/primers which comprises or consists of, or comprises or consists of a sequence complementary to SEQ ID NO: 1 to SEQ ID NO: 28. In some embodiments, the labeled probe-based hybridization analysis assay comprises a NanoString assay. In some embodiments, the measuring of gene activity comprises measuring expression of mRNA levels.

In some embodiments, a cancer is ovarian and/or endometrial cancer. In certain embodiments, a cancer is not ovarian and/or endometrial cancer.

Also disclosed herein is a method of treating cancer in a patient, the method comprising administering an effective amount of an ET and a CDK4/6 inhibitor to the patient after determining the patient has a wild-type ERα protein.

In some embodiments, a wild-type ERα protein activity is determined by measuring expression levels of least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a wild-type ERα protein activity is determined by measuring expression levels of least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a wild-type ERα protein activity is determined by measuring expression levels of least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a wild-type ERα protein activity is determined by measuring expression levels of least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

In some embodiments, a wild-type ERα protein activity is determined by measuring expression levels of genes: ADCY1, GREB1, MYB, NPY1R, PGR, and TFF1. In some embodiments, a wild-type ERα protein activity is determined by measuring expression levels of genes: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, the method further comprises measuring expression levels of one or more internal controls. In some embodiments, the one or more internal controls comprise B2M, GAPDH, PSMC4, and/or PUM1.

In some embodiments, a wild-type ERα protein activity is determined by measuring expression levels of target genes relative to a control, and is determined using a nucleotide quantification assay. In some embodiments, the nucleotide quantification assay is a gene chip assay or RNA sequencing. In some embodiments, the nucleotide quantification assay comprises a labeled probe-based hybridization analysis assay. In some embodiments, the assay comprises one or more targeting probe/primers which comprises or consists of, or comprises or consists of a sequence complementary to SEQ ID NO: 1 to SEQ ID NO: 28. In some embodiments, the labeled probe-based hybridization analysis assay comprises a NanoString assay. In some embodiments, the measuring of gene activity comprises measuring expression of mRNA levels.

Also disclosed herein is a method for treating cancer in a patient, the method comprising administering an effective amount of a CDK4/6 inhibitor to the patient after determining the patient has a mutant or translocated ERα protein resulting in an upregulation of expression of at least six genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, an ERα protein mutation or translocation status is determined by identifying upregulation of expression of at least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, an ERα protein mutation or translocation status is determined by identifying upregulation of expression of at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, an ERα protein mutation or translocation status is determined by identifying upregulation of expression of at least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, an ERα protein mutation or translocation status is determined by identifying upregulation of expression of at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

Also disclosed herein is a method for treating cancer in a patient, the method comprising administering an effective amount of a CDK4/6 inhibitor to the patient after determining whether the patient has a mutant or translocated ERα protein, wherein ERα protein activity is determined by measuring a biological sample from the patient for estrogen response gene expression and/or epithelial to mesenchymal transition (EMT) gene expression, and (A) when the ESR1 gene locus is mutated the therapeutic regimen comprises CDK4/6 inhibitors; or (B) when the ESR1 gene locus is WT, the therapeutic regimen comprises CDK4/6 inhibitors and ET (that is one or more of a SERM, an aromatase inhibitor, and/or a SERD).

In some embodiments, estrogen response gene expression and/or EMT gene expression is determined by identifying upregulation of expression of at least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, estrogen response gene expression and/or EMT gene expression is determined by identifying upregulation of expression of at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, estrogen response gene expression and/or EMT gene expression is determined by identifying upregulation of expression of at least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, estrogen response gene expression and/or EMT gene expression is determined by identifying upregulation of expression of at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

Also disclosed herein is a kit comprising oligonucleotides capable of hybridizing to, and facilitating expression level determination of, eight genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a kit comprises oligonucleotides capable of hybridizing to, and facilitating expression level determination of, twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a kit comprises oligonucleotides capable of hybridizing to, and facilitating expression level determination of, sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a kit comprises oligonucleotides capable of hybridizing to, and facilitating expression level determination of, twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a kit comprises oligonucleotides capable of hybridizing to, and facilitating expression level determination of, eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

In some embodiments, a kit comprises oligonucleotides bound to a substrate.

Also disclosed herein is a composition comprising oligonucleotides hybridized to transcripts from at least eight genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a composition comprises oligonucleotides hybridized to transcripts from at least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a composition comprises oligonucleotides hybridized to transcripts from at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a composition comprises oligonucleotides hybridized to transcripts from at least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a composition comprises oligonucleotides hybridized to transcripts from at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

In some embodiments, a composition comprises oligonucleotides bound to a substrate.

Also disclosed herein is a method of detecting ET-resistant ER+ MBC, comprising measuring the level of expression for at least six genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ET-resistant ER+ MBC detection is determined by measuring expression levels of at least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ET-resistant ER+ MBC detection is determined by measuring expression levels of at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ET-resistant ER+ MBC detection is determined by measuring expression levels of at least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ET-resistant ER+ MBC detection is determined by measuring expression levels of at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

Also disclosed herein is a method of diagnosing ET-resistant ER+ MBC, comprising measuring the level of expression for at least six genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ET-resistant ER+ MBC diagnosis is determined by measuring expression levels of at least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ET-resistant ER+ MBC diagnosis is determined by measuring expression levels of at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ET-resistant ER+ MBC diagnosis is determined by measuring expression levels of at least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, ET-resistant ER+ MBC diagnosis is determined by measuring expression levels of at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, ET-resistant ER+ MBC diagnosis is determined by measuring expression levels of genes: ADCY1, GREB1, MYB, NPY1R, PGR, and TFF1. In some embodiments, ET-resistant ER+ MBC diagnosis is determined by measuring expression levels of genes: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, the method further comprises measuring expression levels of one or more internal controls. In some embodiments, the one or more internal controls comprise B2M, GAPDH, PSMC4, and/or PUM1. In some embodiments, measuring gene expression levels comprises measuring mRNA expression levels.

Also disclosed herein are methods of detecting active ESR1 gene fusions in tissue samples, comprising measuring the level of expression for at least six genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, active ESR1 gene fusion detection is determined by measuring expression levels of at least twelve genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, active ESR1 gene fusion detection is determined by measuring expression levels of at least sixteen genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, active ESR1 gene fusion detection is determined by measuring expression levels of at least twenty-four genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, active ESR1 gene fusion detection is determined by measuring expression levels of at least eighteen genes selected from: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, active ESR1 gene fusion detection is determined by measuring expression levels of genes: ADCY1, GREB1, MYB, NPY1R, PGR, and TFF1. In some embodiments, active ESR1 gene fusion detection is determined by measuring expression levels of genes: CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2. In some embodiments, a method further comprises measuring expression levels of one or more internal controls. In some embodiments, one or more internal controls comprise B2M, GAPDH, PSMC4, and/or PUM1. In some embodiments, the tissue is a breast tissue, ovarian tissue, endometrial tissue, cervical tissue, and/or metastatic tissue. In some embodiments, the active ESR1 gene fusion is a constitutively active ESR1 gene fusion. In some embodiments, measuring gene expression levels comprises measuring mRNA expression levels.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 A-B, depict in-frame ESR1-exon 6 (−e6) fusions identified in estrogen receptor positive (ER+), metastatic breast cancer (MBC) patients. (A) Circos plot depicting estrogen receptor 1 alpha (ERα; encoded by the ESR1 gene) fusion events identified from ER+ MBC patients. The ESR1 gene is connected to its 3′ partner genes with lines. (B) In-frame ESR1 fusions in ER+ MBC possess a common structure whereby the first 6 exons (two untranslated exons and four coding exons in grey, exons 3-6) of ESR1 fuse in-frame to 3′ sequences from partner genes. Key for domains in the wild type (WT) ERα protein: AF1: activation function 1 domain; DBD: DNA-binding domain; Hinge: domain connecting DBD and LBD; and LBD: ligand-binding domain. Pink boxes in partner proteins mediate protein-protein interactions, including WW binding motifs, SH3 binding motifs, a PDZ domain, a conserved motif 2 (CM2), a phospho-tyrosine interaction domain (PID), an interaction with Glycogen Synthase 1 region (GYS1), and a LXXLL motif found in coactivators. Green boxes represent known transcriptional activation domains (TADs). The brown box represents the FAM75 domain of unknown function. Blue domains have enzymatic activities, including substrate binding site (Sub), catalytic site, three manganese binding sites (Mn) and an S-adenosylmethionine-dependent methyltransferase domain (SAM). The grey box labeled NLS represents a nuclear localization signal.

FIG. 2 A-H, describe how ESR1 fusion proteins can drive ET-resistant growth and promote hormone-independent motility and invasion of ER+ breast cancer cells. (A) Immunoblotting of ERα and ESR1 fusion proteins with an N-terminal ERα antibody in lysates made from hormone-deprived stable T47D cells in the presence or absence of 100 nM fulvestrant. Asterisks indicate ER fusion proteins. GAPDH serves as a loading control. The dashed line indicates two separate blots that were conducted at the same time. The representative image is from three independent experiments. (B) Cell growth was assayed in hormone-deprived stable cells (mean±SEM, n=3). One-way ANOVA followed by Dunnett's multiple comparisons test was used to compare data of hormone-deprived ESR1 fusion expressing cells to yellow fluorescent protein (YFP) control cells in the vehicle (+DMSO) group. Two-way ANOVA followed by Bonferroni's test was used for multiple comparisons for each stable cell line after 100 nM fulvestrant treatment in the presence or absence of 10 nM estradiol (E2). *p<0.05, **p<0.01, ***p<0.001, and ****p<0.0001. (C) Cell motility was assayed using scratch wound assays in hormone-deprived stable T47D cells, treated with 50 ng/ml mitomycin C to block proliferation (mean±SEM, n=3). Cells are pseudo-colored orange to aid visualization. One-way ANOVA followed by Dunnett's multiple comparisons test was used to compare each stable T47D cell line to YFP control cells (ns: not significant). These results demonstrate that in-frame ESR1 fusions drive ET-resistant cell growth and promote hormone-independent cell motility. (D) Immunoblotting of ERα and ESR1 fusion proteins with an N-terminal ERα antibody, Snail or E-cad in hormone-deprived stably expressing MCF7 cells in the presence or absence of 100 nM fulvestrant. GAPDH serves as a loading control. Asterisks indicate ESR1 fusion proteins. The dashed line indicates two separate blots that were conducted at the same time. The representative image is from two independent experiments. (E) Cell growth was assayed in stably expressing T47D cell controls (YFP, ESR1-e6, or ESR1-WT) or eight ESR1 fusion proteins (mean±SEM, n=3). Two-way ANOVA test was used to compare each stable cell after 10 nM estradiol treatment (−E2 vs+E2, ⁺p<0.05, ⁺⁺p<0.01, ⁺⁺⁺p<0.001, and ⁺⁺⁺⁺p<0.0001). One-way ANOVA followed by Dunnett's multiple comparisons test was used to compare each ESR1 fusion expressing cell line to YFP control cells in the presence of E2 and fulvestrant (+E2, +Fulvestrant) (^{#}p<0.05, ^{##}p<0.01, ^{###}p<0.001 and ^{###}p<0.0001). (F) Cell growth was assayed in MCF7 cells stably expressing various ESR1 constructs (mean±SEM, n=3). One-way ANOVA followed by Dunnett's multiple comparisons test was used to compare each stable MCF7 cell line to YFP control cells. In the absence of estradiol (−E2), ****p<0.0001. In the presence of E2 and fulvestrant (+E2, +Fulvestrant), ^{#}p<0.05, ^{##}p<0.01, ^{###}p<0.001 and ^{####}p<0.0001. Two-way ANOVA was used to compare each stable cell line after 10 nM estradiol treatment (−E2 vs+E2, ⁺p<0.05, ⁺⁺p<0.01, ⁺⁺⁺p<0.001, and ⁺⁺⁺⁺p<0.0001). (G) Cell motility was assayed using scratch wound assays in hormone-deprived stably expressing MCF7 cells, treated with 200 ng/ml mitomycin C to block proliferation and quantified by the relative wound density (mean±SEM, n=3). One-way ANOVA followed by Dunnett's multiple comparisons test was used to compare each cell line to YFP control cells. Cells are pseudo-colored orange to aid visualization. (H) Active in-frame ESR1 fusions promote hormone-independent cell invasion. Cell invasion was assayed in hormone-deprived stably expressing T47D cells, in a similar manner to the scratch wound assay except that cells were first plated on a Matrigel-coated plate, as described in the Methods. Top panel, IncuCyte images were recorded. Bottom panel, relative wound densities (%) were calculated and plotted (mean±SEM, n=3). One-way ANOVA followed by Dunnett's multiple comparisons test was used to compare each cell line to YFP control cells. Wound regions are pseudo-colored green to aid visualization.

FIG. 3 A-I, describe how active ESR1 fusion proteins can upregulate expression of estrogen response and EMT genes. (A) Heatmap showing unsupervised hierarchical clustering of differently expressed genes in the T47D RNA-Seq data. Scale bar indicates row Z sores. (B and C) Active ESR1 fusions upregulate expression of two clusters of estrogen response (early and late) and epithelial to mesenchymal transition (EMT)-related genes as indicated. (D and E) Expression of estrogen response genes (GREB1, TFF1 and PGR) and EMT-related genes (VCAN and SNAI1) were measured by RT-qPCR in estrogen (E2)-deprived T47D cells treated with vehicle (+DMSO) or 100 nM fulvestrant (+Fulv) in the absence (−E2) or presence (+E2) of 10 nM E2 for 48 hours. Values were normalized to GAPDH mRNA and relative expression was calculated as fold change to YFP, −E2 (mean±SEM, n=3). One-way ANOVA followed by Dunnett's multiple comparisons test was used to compare each E2-deprived T47D cell line to YFP control cells (*p<0.05, **p<0.01, ***p<0.001, and ****p<0.0001). Two-way ANOVA followed by Bonferroni's test was used for multiple comparisons for each stable cell after 100 nM fulvestrant treatment. (F) Snail and E-cadherin proteins were measured by immunoblotting in E2-deprived cells treated with or without 100 nM fulvestrant (Fulv). GAPDH protein served as a loading control. The dashed line indicates two separate blots that were conducted at the same time. The representative image is from three independent experiments. (G) ESR1 fusion proteins localize to the nucleus. Immunofluorescence staining with anti-HA tag antibody (red) on hormone-deprived stably expressing T47D cells showed nuclear localization of all eight HA-tagged ESR1 fusion constructs. Nuclei were labeled with DAPI (blue). Representative images from two independent experiments are shown. Magnification 40×; Scale bar, 50 m. (H) Hormone-deprived T47D cells stably expressing different ESR1 constructs were treated with or without 100 nM E2 for 45 min. Cell lysates were then immunoprecipitated with an anti-HA antibody or mouse IgG control and then blotted with a N-terminal ERα antibody. Blotting with a C-terminal ERα antibody only detected ER-WT, and no co-immunoprecipitation with any ESR1 fusion protein, thereby demonstrating that ESR1 fusions do not form heterodimers with endogenously expressed ERα. Representative images are from two independent experiments. The dashed line indicates two separate blots that were conducted at the same time. (I) Whole cell lysates (1% inputs for co-IP) were detected by immunoblotting with N- and C-terminal ERα antibodies, along with GAPDH as the loading control.

FIG. 4 A-D, depict identification of active ESR1 fusions programming a unique, 24-gene transcriptional signature. (A) Workflow to identify the gene signature to predict active fusions (FC: fold change. FDR: false discovery rate. PDX: patient-derived xenograft). (B) Venn diagram showing overlap of upregulated genes by active ESR1 fusions compared to inactive fusions or control cells. Table below shows the top three Hallmark gene sets enriched in the candidate genes. (C) Scatter plot showing signature scores of active ESR1 fusions (ESR1-e6>YAP1, ESR1-e6>PCDH11X, ESR1-e6>SOX9, and ESR1-e6>ARNT2-e18) compared to inactive fusions (ESR1-e6>GYG1, ESR1-e6>PCMT1 and ESR1-e6>ARID1B) and control cells (YFP, ESR1-e6 and ESR1-WT) all minus E2 in the training set. A two-tailed t test was used to calculate statistical significance. (D) Receiver operating characteristic (ROC) curve showing performance of the 24-gene signature to classify activities of ESR1 fusions in the original training set. A cutoff of 0.3283 for mean signature score was defined.

FIG. 5 A-I, describe how a 24-gene transcriptional signature, termed “Mutant or Translocated Estrogen Receptor Alpha” (MOTERA) signature predicts activity of additional ESR1 fusions identified in ER+ MBC patients. (A) Seven additional ESR1-e6 fusions identified in Priestley et al. (27) are illustrated. These in-frame fusions possess a common structure as shown in FIG. 1B. Pink boxes represent protein-protein interactions, including the Per-Arnt-Sim (PAS) domain, PAC motif, LXXLL motif, Class A specific domain (CAD), Threonine-rich domain (Thr rich), Methionine-rich domain (Met rich), PDZ domain and PABPC1-interacting motif-2 (PAM2). Green boxes either represent transcriptional activation domains (TADs) or LIM zinc-binding (LIM) domains that provide coactivator function for LPP. The grey box represents a nuclear export signal (NES) in LPP. Red boxes represent the bHLH DNA binding domain and the RNA Recognition Motif (RRM). (B) Heatmap showing unsupervised hierarchical clustering for expression of the 24-gene signature in T47D cells expressing additional ESR1 fusions and LBD point mutations (Y537S and D538G). Scale bar indicates row Z scores. (C) Left panel: Scatter plot showing signature scores of ESR1 mutations (including fusions and LBD point mutations) and YFP control cells expressing endogenous ERα. Two-tailed t test was used to compare scores. The ESR1-GRIP1 fusion was the only active fusion that was below the score cutoff. Right panel: Confusion matrix to measure the performance of the signature to predict the activities of ESR1 fusions. Accuracy is the proportion of correctly predicted events in all cases. Sensitivity is the ability of the signature to predict an active fusion event to be active. Specificity is the ability of the signature to predict an inactive fusion event to be inactive. These results demonstrate that additional ESR1 fusions identified in ER+ MBC patients drive ET-resistant growth and promote hormone-independent motility of ER+ breast cancer cells. (D and E) Immunoblotting of ERα and ESR1 fusion proteins with an N-terminal ERα antibody in hormone-deprived stably expressing T47D and MCF7 cells in the absence or presence of 100 nM fulvestrant. Asterisks indicate ESR1 fusion proteins. GAPDH serves as a loading control. The dashed line indicates two separate blots that were conducted at the same time. The representative image is from 2-3 independent experiments. (F and G) Cell growth was assayed in hormone-deprived cells stably expressing an ESR1 construct (mean±SEM, n=3). One-way ANOVA followed by Dunnett's multiple comparisons test was used to compare data of hormone-deprived ESR1 fusion expressing cells to YFP control cells in vehicle control (+DMSO) group. Two-way ANOVA followed by Bonferroni's test was used for multiple comparisons for each stable cell line after 100 nM fulvestrant treatment in the presence or absence of 10 nM E2. *p<0.05, **p<0.01, ***p<0.001, ****p<0.0001. (H) Cell motility was assayed using scratch wound assays in hormone-deprived T47D cells stably expressing an ESR1 construct and treated with 50 ng/ml mitomycin C to block proliferation (mean±SEM, n=3). One-way ANOVA followed by Dunnett's multiple comparisons test was used to compare each stable T47D cell line to YFP control cells (ns: not significant). Cells are pseudo-colored orange to aid visualization. (I) Inactive ESR1-TCF12 fusion protein binds estrogen response element (ERE)-containing DNA like active ESR1 fusion proteins. Nuclear extracts of T47D cell lines expressing YFP, ESR1-e6>YAP1, ESR1-e6>SOX9, ESR1-e6>CLINT1, or ESR1-e6>TCF12 were prepared as described in the Methods section. All ESR1 fusion-expressing cells were treated with 100 nM fulvestrant (Fulv) overnight to reduce the endogenous ERα level and thus its competition for binding to the EREs. ERE DNA pulldowns were done as described in the Methods section. Top panel, Immunoblotting of ERα and ESR1 fusion proteins with an N-terminal ERα antibody reveals levels in 2% of starting inputs (left) and what was bound to 30% of the final 4×ERE DNA beads (right). Asterisks indicate ER fusion proteins. Bottom panel, Immunoblotting of DNA-PK catalytic subunit (DNA-PKcs) serves as a loading control. The dashed line indicates two separate blots that were conducted at the same time.

FIG. 6, depicts the growth of 20 ER+ patient-derived xenografts (PDX) tumors in xenografted mice in the absence and presence of E2. Volumes of 20 ER+ PDX tumors were measured in ovariectomized SCID/beige mice supplemented with or without 8 g/ml E2 in the drinking water (mean±SEM, n=7-16 per PDX line per arm). PDX tumors were categorized based on ESR1 status (mutations listed or wild-type, wt) and E2 dependency for growth (E2-independent, E2-suppressed, E2-partially dependent and E2-dependent).

FIG. 7 A-D, describe how the MOTERA signature predicts activity of ESR1 fusions/point mutations in ER+ PDX tumors and in MBC patients. (A) Heatmap showing the expression of the 24-gene signature in 20 ER+ PDX tumors. Scale bar indicates row Z scores. CALCR and KRT13 in the signature were missing in the PDX RNA-Seq data, so they were not included in the heatmap. (B) Left panel: Scatter plot showing mean signature scores of ESR1 mutations (including the ESR1-YAP1 fusion and LBD point mutations) and ESR1-WT expressing tumors. One-way ANOVA with Dunnett's multiple comparisons test was used to calculate statistical significance. Right panel: Confusion matrix to measure the performance of the signature to predict the presence of ESR1 mutations. Accuracy is the proportion of correctly predicted events in all cases. Sensitivity is the ability of the signature to predict an ESR1 mutation to be a mutant. Specificity is the ability of the signature to predict an ESR1-WT to be wild-type. (C) Scatter plot showing mean signature scores of MBC patient tumors expressing ESR1 mutations versus ESR1-WT in the MET500 cohort (9). Two-tailed t test was used to compare scores. (D) ROC curve for the 24-gene signature performance to differentiate ESR1 mutations from ESR1-WT in the MET500 cohort. The area under the curve (AUC) is the probability that the signature ranks a randomly chosen ESR1 mutation higher than a randomly chosen WT ESR1 (100% is the best test and the dashed diagonal line illustrates the performance of a random signature).

FIG. 8 A-B, depicts NanoString-based MOTERA signature prediction activity of ESR1-e6 fusion proteins expressed in T47D cells. (A) Active ESR1 fusions showed significantly higher MOTERA scores than YFP controls and inactive ESR1 fusions in T47D cells, detected by NanoString assay. (B) Heatmap depiction of expression of 24 genes in an exemplary MOTERA signature in T47D cells expressing listed ESR1 fusions (ESR1-e6>TCF12; ESR1-e6>PCMT1; ESR1-e6>YAP1; ESR1-e6>SOX9; and ESR1-e6>CLINT1).

FIG. 9 A-B, depicts NanoString-based MOTERA signature prediction activity of ESR1-e6 fusion proteins expressed in patient derived xenograft (PDX) tumors. (A) ESR1-e6>YAP1 fusion and ESR1-Y537S point mutations showed significantly higher MOTERA scores than WT ESR1 in PDX tumors, detected by NanoString assay. (B) Heatmap depiction of expression of 24 genes in an exemplary MOTERA signature in PDX tumors.

FIG. 10 A-D, depicts NanoString-based 6-gene signature prediction activity of ESR1-e6 fusion proteins expressed in T47D cells and PDX tumors. (A) Active ESR1 fusions showed significantly higher MOTERA scores than YFP controls and inactive ESR1 fusions in T47D cells, detected by NanoString assay. (B) Heatmap depiction of expression of a 6 gene signature (ADCY1, GREB1, MYB, NPY1R, PGR, and TFF1) in T47D cells expressing listed ESR1 fusions (ESR1-e6>YAP1; ESR1-e6>SOX9; ESR1-e6>CLINT1; ESR1-e6>PCMT1; and ESR1-e6>TCF12). (C) ESR1-e6>YAP1 fusion expressed in WHIM18 PDX and ESR1-Y537S point mutation expressed in WHIM20 PDX showed significantly higher MOTERA scores than WT ESR1 in WHIM9 PDX tumors, detected by NanoString assay. All mice in this experiment were ovariectomized to reduce the effect of E2. (D) Heatmap depiction of expression of a 6 gene signature (ADCY1, GREB1, MYB, NPY1R, PGR, and TFF1) enriched in WHIM 18 and WHIM20 (ESR1 variant expressing) versus WHIM9 (ESR1-WT expressing) PDX tumors.

### DETAILED DESCRIPTION

Use of the one or more compositions may be employed based on methods described herein. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

“Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, and/or duration of response in a patient or a group of patients susceptible to or diagnosed with a cancer.

In certain aspects, prognosis is an estimation of the likelihood of metastasis-free survival of said patient over a predetermined period of time, e.g., over a period of 5 years.

In further aspects, prognosis is an estimation of the likelihood of death due to disease of said patient over a predetermined period of time, e.g., over a period of 5 years.

The term “recurrence” refers to the detection of breast cancer in form of metastatic spread of tumor cells, local recurrence, contralateral recurrence or recurrence of breast cancer at any site of the body of the patient after breast cancer had been substantially undetectable or responsive to treatments.

As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, and/or duration of response in a patient or a group of patients susceptible to or diagnosed with a cancer.

By “gene” is meant any polynucleotide sequence or portion thereof with a functional role in encoding or transcribing a protein or regulating other gene expression. The gene may consist of all the nucleic acids responsible for encoding a functional protein or only a portion of the nucleic acids responsible for encoding or expressing a protein. The polynucleotide sequence may contain a genetic abnormality within exons, introns, initiation or termination regions, promoter sequences, other regulatory sequences or unique adjacent regions to the gene.

As used herein, “treatment” or “therapy” is an approach for obtaining beneficial or desired clinical results. This includes: reduce the number of cancer cells; reduce the tumor size; inhibit (i.e., slow to some extent and/or stop) cancer cell infiltration into peripheral organs; inhibit (i.e., slow to some extent and/or stop) tumor metastasis; inhibit, to some extent, tumor growth; and/or relieve to some extent one or more of the symptoms associated with the disorder, shrinking the size of the tumor, decreasing symptoms resulting from the disease, increasing the quality of life of those suffering from the disease, decreasing the dose of other medications required to treat the disease, delaying the progression of the disease, and/or prolonging survival of patients.

The term “therapeutically effective amount” refers to an amount of the drug that may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “overexpress”, “overexpression”, “overexpressed”, “up-regulate”, or “up-regulated” interchangeably refer to a biomarker that is transcribed or translated at a detectably greater level, usually in a cancer cell, in comparison to a non-cancer cell or cancer cell that is not associated with the worst or poorest prognosis. The term includes overexpression due to transcription, post transcriptional processing, translation, post-translational processing, cellular localization, and/or RNA and protein stability, as compared to a non-cancer cell or cancer cell that is not associated with the worst or poorest prognosis. Overexpression can be detected using conventional techniques for detecting mRNA (i.e., RT-PCR, PCR, hybridization) or proteins (i.e., ELISA, immunohistochemical techniques, mass spectrometry). Overexpression can be 10%, 20%, 30%, 40%, 50%, 60%, 70%, 80%, 90% or more in comparison to a normal cell or cancer cell that is not associated with the worst or poorest prognosis. In certain instances, overexpression is 1-fold, 2-fold, 3-fold, 4-fold 5, 6, 7, 8, 9, 10, or 15-fold or even higher levels of transcription or translation in comparison to a non-cancer cell or cancer cell that is not associated with the worst or poorest prognosis.

“Biological sample” includes sections of tissues such as biopsy and autopsy samples, and frozen sections taken for histologic purposes. Such samples include breast cancer tissues, cultured cells, e.g., primary cultures, explants, and transformed cells. A biological sample is typically obtained from a mammal, such as a primate, e.g., human.

A “biopsy” refers to the process of removing a tissue sample for diagnostic or prognostic evaluation, and to the tissue specimen itself. Any biopsy technique known in the art can be applied to the diagnostic and prognostic methods of the present disclosure. The biopsy technique applied will depend on the tissue type to be evaluated (e.g., breast), the size and type of the tumor, among other factors. Representative biopsy techniques include, but are not limited to, excisional biopsy, incisional biopsy, needle biopsy, and surgical biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy”, or a “fine-needle aspiration biopsy” which generally obtains a suspension of cells from within a target tissue. Biopsy techniques are discussed, for example, in Harrison's Principles of Internal Medicine, 2005. Obtaining a biopsy includes both direct and indirect methods, including obtaining the biopsy from the patient or obtaining the biopsy sample after it is removed from the patient.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present disclosure will become apparent from the following detailed description. It should be understood, however, that the detailed description and the specific examples, while indicating specific embodiments, are given by way of illustration only, since various changes and modifications within the spirit and scope of the immediate disclosure will become apparent to those skilled in the art from this detailed description.

### I. Estrogen Receptors and Other Nuclear Hormone Receptors

Intracellular receptors (IRs) form a class of structurally-related genetic regulators scientists have named “ligand-dependent transcription factors” (R. M. Evans, Science, 240:889, 1988). Steroid receptors are a recognized subset of the IRs, including androgen receptor (AR), progesterone receptor (PR), estrogen receptor (ER), glucocorticoid receptor (GR), and mineralocorticoid receptor (MR).

Estrogen, via binding the estrogen receptor (ER), plays a major role in regulating the growth and differentiation of normal breast epithelium (Pike et al. Epidemiologic Reviews (1993) 15(1):17-35; Henderson et al. Cancer Res. (1988) 48:246-253). It stimulates cell proliferation and regulates the expression of other genes, including the progesterone receptor (PR). PR then mediates the mitogenic effect of progesterone, further stimulating proliferation (Pike et al., 1993; Henderson et al., 1988). The molecular differences between estrogen receptor (“ER”) negative and ER positive tumors are significant in light of clinical observations which indicate that the nature and biological behavior of ER positive and ER negative tumors are distinct even in the absence of hormonal therapy. For example, ER negative cancers tend to recur sooner and show a different rate of recurrence in distant organ sites compared to ER positive tumors. Clinical observations and molecular profiling data suggest that tumors not expressing both ER and PR represent a different clinical entity in terms of chemotherapy responsiveness. (Colleoni et al., Annals of Oncology 11(8):1057 (2000)). Thus, ER negative and ER positive breast cancers are two distinct disease entities rather than phenotypic variations of the same disease.

The majority of breast cancers (˜70%) are initially diagnosed as estrogen receptor-alpha positive (ERα+) and are dependent on 17-3 estradiol (E2) for growth (1). Thus, endocrine therapies (ET) either induce estrogen deprivation, for example through aromatase inhibition (AI), or directly target the ERα ligand binding domain (LBD) with selective ER modulation (e.g., tamoxifen) or degradation (e.g., fulvestrant) (1). However acquired ET resistance is common, and is often associated with somatic mutations in the gene encoding ERα, ESR1. The most extensively studied examples are point mutations in the LBD that result in ERα proteins with ligand-independent activity. Common examples include Y537S and D538G (2,3). These mutations typically arise after patients have undergone extensive ET and can be present in up to 40% of patients with ERα+ metastatic breast cancer (MBC) (4,5).

Emerging evidence indicates that chromosomal translocations involving the ESR1 gene can also drive ET-resistance through the formation of chimeric transcription factors (TF) with constitutive activity (6,7). The first described example was an ESR1-e6>YAP1 fusion detected by whole genome sequencing and RNA sequencing (RNA-Seq) in samples from a patient with rapid onset ET resistance (6). The fusion protein was encoded by an inter-chromosomal translocation event that brought ESR1 exons 1 to 6 (ESR1-e6) on chromosome (chr) 6q into the YAP1 locus on chr11q thereby replacing the entire LBD with transactivation domain (TAD) sequences from this Hippo pathway transcriptional coactivator (CoA). A PDX established from the patient's tumor (WHIM18) also exhibited ET resistance. We subsequently identified another in-frame exon 6 fusion, ESR1-e6>PCDH11X in a male patient with ER+ MBC as a result of a chr6q>Xq translocation. This second example was a harbinger of complexity to come, since PCDH11X encodes a protocadherin without known transcriptional functions. Both fusions not only induced ET-resistant tumor growth but also increased lung metastasis in xenograft mice models (7). Another in-frame ESR1-e6 fusion, ESR1-e6>NOP2, was detected in a primary tumor but was found to be transcriptionally inactive (7). This example suggested that the mere presence of an ESR1-e6 fusion that generates a stable chimeric protein is insufficient evidence that an ET-resistance driver has been identified.

Multiple additional ESR1-e6 fusions have now been identified from ER+ MBC patients. In a study by Lee et al., three ESR1-e6 fusions (ESR1-e6>DAB2, ESR1-e6>GYG1, and ESR1-e6>SOX9) were shown to activate an estrogen response element (ERE)-driven luciferase reporter construct in transfected HEK293 cells (8). In the MET500 study (9), three in-frame fusions (ESR1-e6>ARNT2-e18, ESR1-e6>PCMT1, and ESR1-e6>ARID1B) were identified in samples from MBC patients and were provided to us for functional studies in a pre-publication personal communication. To further investigate the significance of in-frame ESR1-e6 fusion genes, each example was screened for ET-resistance induction in vitro, defined as E2-independent and fulvestrant-resistant growth and increased motility, in two ER+ breast cancer cell lines (T47D and MCF7). RNA-Seq was undertaken to understand the transcriptional reprogramming induced by active ESR1 fusions. A series of 66 biomarkers were found to be upregulated when compared to control samples and inactive fusion control samples. In some embodiments, a combination of a subset of these biomarkers is utilized to predict the presence of an active ESR1 fusion. As described herein, an exemplary 24-gene signature that was characteristic of the presence of an active ESR1 fusion as compared to an inactive fusion or wild-type (WT) ESR1 was determined. This signature was validated in ER+ PDXs and in clinical samples, activating LBD point mutations were discovered to also induce this gene expression signature. These data suggest that despite the remarkable diversity of mutations in the ESR1 gene, these somatic events converge on a common pathogenic transcriptional reprogramming mechanism to drive poor outcome and ET-resistance in MBC. As described herein, in some embodiments, this transcriptional reprogramming is utilized to direct treatment regimens and facilitate positive patient outcomes. In some embodiments, identified transcriptional reprogramming is indicative of active ESR1 fusions and/or ESR1 ligand-binding domain (LBD) point mutations.

### II. ER Antagonists and Inhibitors

The genes identified as ER-responsive or ET non-responsive were evaluated in the context of one or more ER antagonists. Certain embodiments concern treatment with one or more ER antagonists for cancer. Certain embodiments concern treatment with one or more non-endocrine therapies for cancer. A number of ER antagonists have been identified. ER antagonists/inhibitors include, but are not limited to, Aromatase Inhibitors (AIs) such as letrozole and anastrozole, Selective Estrogen Receptor Down Regulators (SERDs) such as fulvestrant, and Selective Estrogen Receptor Modulators (SERMs) such as tamoxifen.

### III. Biomarkers and Evaluating Levels of Biomarkers

Biomarkers for identifying effective treatment for human breast cancer patients are provided. It is contemplated that these biomarkers may be evaluated based on their gene products. In some embodiments, the gene product is an RNA transcript. In some embodiments, the gene product is a mRNA transcript. In other embodiments, the gene product is a protein expressed by a mRNA transcript. In other embodiments, the gene product is an evaluation of surrogate genes or gene targets.

In certain aspects a meta-analysis of expression or activity can be performed. In statistics, a meta-analysis combines the results of several studies that address a set of related research hypotheses. This is normally done by identification of a common measure of effect size, which is modeled using a form of meta-regression. Generally, three types of models can be distinguished in the literature on meta-analysis: simple regression, fixed effects meta-regression and random effects meta-regression. Resulting overall averages when controlling for study characteristics can be considered meta-effect sizes, which are more powerful estimates of the true effect size than those derived in a single study under a given single set of assumptions and conditions. A meta-gene expression value, in this context, is to be understood as being the median of the normalized expression of a marker gene or activity. Normalization of the expression of a marker gene is preferably achieved by dividing the expression level of the individual marker gene to be normalized by the respective individual median expression of this marker genes, wherein said median expression is preferably calculated from multiple measurements of the respective gene in a sufficiently large cohort of test individuals. The test cohort preferably comprises at least 3, 10, 100, 200, 1000 individuals or more including all values and ranges thereof. Dataset-specific bias can be removed or minimized allowing multiple datasets to be combined for meta-analyses (See Sims et al. BMC Medical Genomics (1:42), 1-14, 2008, which is incorporated herein by reference in its entirety).

Calculation of a meta-gene expression value is performed by: (i) determining the gene expression value of at least two, preferably more genes (ii) “normalizing” the gene expression value of each individual gene by dividing the expression value with a coefficient which is approximately the median expression value of the respective gene in a representative breast cancer cohort (iii) calculating the median of the group of normalized gene expression values.

A gene shall be understood to be specifically expressed in a certain cell type if the expression level of the gene in the cell type is at least about 2-fold, 5-fold, 10-fold, 100-fold, 1000-fold, or 10000-fold higher (or any range derivable therein) than in a reference cell type, or in a mixture of reference cell types. Reference cell types include but are not limited to, non-cancerous breast tissue cells or a heterogeneous population of breast cancers.

In certain algorithms a suitable threshold level is first determined for a marker gene. The suitable threshold level can be determined from measurements of marker gene expression in multiple individuals from a test cohort. The median expression of marker gene in said multiple expression measurements is taken as the suitable threshold value.

Comparison of multiple marker genes with a threshold level can be performed as follows: 1) The individual marker genes are compared to their respective threshold levels. 2) The number of marker genes, the expression level of which is above their respective threshold level, is determined. 3) If a marker genes is expressed above its respective threshold level, then the expression level of the marker gene is taken to be “above the threshold level”.

“A sufficiently large number”, in this context, means preferably 20%, 30%, 40%, 50%, 60%, 70%, 80%, 90%, or 95% of the marker genes used.

In certain aspects, the determination of expression levels is on a substrate that allows evaluation of RNA molecule levels from a given sample, such as a gene chip, for example but not limited to Affymetrix™ gene chip, NanoString nCounter™, Illumina BeadChip™, etc. In some embodiments, determination of expression levels comprises labeled probe-based hybridization analysis. In certain embodiments, the labeled probe-based hybridization analysis measures RNA levels. In certain embodiments, the labeled probe-based hybridization analysis measures mRNA levels. In certain embodiments, the label in a labeled probe comprises a chromogenic label, fluorescent label, epitope label, and/or hapten label. In certain embodiments, the labeled probe-based hybridization analysis comprises a NanoString assay.

In some embodiments, determination of expression levels is by RNA sequencing. In some embodiments, provided RNA sequencing technologies are commercially available, including but not limited to products from Illumina, Thermo Fisher Scientific, Oxford Nanopore, Agilent Technologies, Inc., BGI, PerkinElmer Inc., QIAGEN, Eurofins Scientific, F. Hoffmann-La Roche Ltd, Takara Bio Inc., GENEWIZ, Inc., Hamilton Company, Macrogen, Zymo Research, and Tecan Genomics, Inc.

In some embodiments, determination of expression levels is by targeted RNA quantification. In some embodiments, provided targeted RNA quantification technologies are commercially available, including but not limited to products from Thermo Fisher Scientific (e.g., QuantiGene RNA Assays), Qiagen (e.g., QIAseq Targeted RNA Panels), Illumina (e.g., Targeted RNA Sequencing), NanoString (e.g., nCounter), Advanced Cell Diagnostics (e.g., RNAscope), Molecular Instruments (e.g., HCR RNA-FISH), Genotix Biotechnologies (e.g., DaVinci Analyzer), and Biosearch Technologies (e.g., Stellaris RNA FISH), etc.

In other embodiments, the determination of expression levels is done by reverse transcription-quantitative kinetic real time PCR (RT-qPCR).

In other embodiments, the determination of expression levels is done by measuring protein and/or polypeptides instead of RNA, for example by utilizing methods such as immunoblotting, IP-MS/MS, ELISAs, flow cytometry, etc. In some embodiments, the determination of RNA expression levels is done by flow cytometry.

In certain aspects, methods can relate to a system for performing such methods, the system comprising (a) apparatus or device for storing data regarding expression levels of one or more ER-antagonist responsive genes of the patient; (b) apparatus or device for determining expression level of at least one marker gene; (c) apparatus or device for comparing expression level of the first marker gene with a predetermined first threshold value; (d) apparatus or device for determining expression level of at least one second or more marker gene; and (e) computing apparatus or device programmed to provide treatment with a ER antagonist and/or non-endocrine therapy if the data indicates altered expression levels of said first marker gene or activity as compared to the predetermined first threshold value and, alternatively or in concert, expression level of said second or more marker gene is above or below a predetermined second threshold level, wherein the predetermined threshold values are based on expression levels for genes unaltered after exposure to an endocrine therapy.

The person skilled in the art readily appreciates that an unfavorable or poor prognosis can be given or determined if expression level of a first marker gene with a predetermined first threshold value indicates a tumor that is likely to recur or not respond well to standard therapies, including a particular therapy of any kind, such as ET.

Expression patterns can also be compared by using one or more ratios between expression levels of different breast cancer biomarkers. Other suitable measures or indicators can also be employed for assessing the relationship or difference between different expression patterns.

The following biomarkers are provided for implementation with some embodiments discussed herein. All of them designate nucleic acid sequences for the particular gene identifier. Nucleic acid sequences related to these gene designation can be found in the GenBank® sequence databases. In some embodiments, biomarkers include one or more of acyl-CoA oxidase 2 (ACOX2), adenylate cyclase 1 (ADCY1), adrenoceptor alpha 2A (ADRA2A), AF4/FMR2 family member 3 (AFF3), archaelysin family metallopeptidase 1 (AMZ1), beaded filament structural protein 2 (BFSP2), bone morphogenetic protein receptor type 1B (BMPR1B), Homo sapiens chromosome 14 open reading frame 182 (C14orf182), calcitonin receptor (CALCR), coiled-coil domain containing 88A (CCDC88A), CD109 molecule (CD109), CD34 molecule (CD34), carbohydrate sulfotransferase 8 (CHST8), collagen type III alpha 1 chain (COL3A1), cancer/testis associated 62 (CT62), C—X—C motif chemokine ligand 12 (CXCL12), docking protein 7 (DOK7), DS cell adhesion molecule like 1 (DSCAML1), ELOVL fatty acid elongase 2 (ELOVL2), fms related receptor tyrosine kinase 4 (FLT4), formin 1 (FMN1), GATA binding protein 4 (GATA4), GDNF family receptor alpha 1 (GFRA1), gap junction protein alpha 1 (GJA1), growth regulating estrogen receptor binding 1 (GREB1), gremlin 2, DAN family BMP antagonist (GREM2), hes related family bHLH transcription factor with YRPW motif 2 (HEY2), interferon induced transmembrane protein 10 (IFITM10), insulin like growth factor 2 (IGF2), potassium voltage-gated channel subfamily H member 1 (KCNH1), keratin 13 (KRT13), microtubule associated protein tau (MAPT), MAM domain containing glycosylphosphatidylinositol anchor 1 (MDGA1), metallophosphoesterase domain containing 2 (MPPED2), MYB proto-oncogene, transcription factor (MYB), sodium/potassium transporting ATPase interacting 1 (NKAIN1), neuropeptide Y receptor Y1 (NPY1R), neurexophilin 3 (NXPH3), olfactomedin 1 (OLFM1), PDZ domain containing 1 (PDZK1), peptidoglycan recognition protein 2 (PGLYRP2), progesterone receptor (PGR), protein phosphatase 2 regulatory subunit B gamma (PPP2R2C), serine protease 56 (PRSS56), RAS guanyl releasing protein 1 (RASGRP1), RNA binding motif protein 24 (RBM24), regulating synaptic membrane exocytosis 4 (RIMS4), roundabout guidance receptor 3 (ROBO3), semaphorin 3A (SEMA3A), serpin family A member 6 (SERPINA6), serum/glucocorticoid regulated kinase 1 (SGK1), solute carrier family 47 member 1 (SLC47A1), SRY-box transcription factor 5 (SOX5), serine peptidase inhibitor Kazal type 13 (SPINK13), serine peptidase inhibitor Kazal type 4 (SPINK4), serine peptidase inhibitor Kazal type 5 (SPINK5), stanniocalcin 1 (STC1), stanniocalcin 2 (STC2), sushi domain containing 3 (SUSD3), synaptotagmin like 5 (SYTL5), trefoil factor 1 (TFF1), transglutaminase 2 (TGM2), UDP glycosyltransferase family 3 member A2 (UGT3A2), versican (VCAN), WT1 transcription factor (WT1), and zinc finger protein 385B (ZNF385B) genes.

In some embodiments, biomarkers include one or more of carbohydrate sulfotransferase 8 (CHST8), microtubule associated protein tau (MAPT), olfactomedin 1 (OLFM1), PDZ domain containing 1 (PDZK1), RAS guanyl releasing protein 1 (RASGRP1), metallophosphoesterase domain containing 2 (MPPED2), growth regulating estrogen receptor binding 1 (GREB1), MYB proto-oncogene transcription factor (MYB), GDNF family receptor alpha 1 (GFRA1), progesterone receptor (PGR), ELOVL fatty acid elongase 2 (ELOVL2), adenylate cyclase 1 (ADCY1), neuropeptide Y receptor Y1 (NPY1R), trefoil factor 1 (TFF1), acyl-CoA oxidase 2 (ACOX2), serum/glucocorticoid regulated kinase 1 (SGK1), stanniocalcin 2 (STC2), calcitonin receptor (CALCR), keratin 13 (KRT13), versican (VCAN), collagen type III alpha 1 chain (COL3A1), C—X—C motif chemokine ligand 12 (CXCL12), gap junction protein alpha 1 (GJA1), and transglutaminase 2 (TGM2) genes.

In some embodiments, biomarkers include one or more of adenylate cyclase 1 (ADCY1), growth regulating estrogen receptor binding 1 (GREB1), MYB proto-oncogene transcription factor (MYB), neuropeptide Y receptor Y1 (NPY1R), progesterone receptor (PGR), and trefoil factor 1 (TFF1) genes.

One or more of the biomarkers can be used to determine whether a human patient with breast cancer should be treated with one or more ER antagonists or inhibitors and/or non-endocrine therapies (with or without additional cancer therapy). The expression pattern of these biomarkers in breast cancer cells may be used to evaluate a patient to determine whether they are likely to respond to an ER antagonist/inhibitor or likely not to respond to an ER antagonist/inhibitor. The likeliness of a response for the patient may be considered with respect to an individual that lacks the particular expression pattern of the patient.

In some embodiments, expression levels of breast cancer biomarkers can be compared to reference expression levels using various methods. In some embodiments, reference levels can be determined using expression levels of a reference based on all types of breast cancer patients, or all types of breast cancer patients determined to be ER antagonist responsive. In some embodiments, reference levels can be based on an internal reference such as a gene that is expressed ubiquitously. In some embodiments, comparison can be performed using the fold change or the absolute difference between the expression levels to be compared. In some embodiments, one or more breast cancer biomarkers can be used in the comparison. It is contemplated that 1, 2, 3, 4, 5, 6, 7, 8, 9, 10, 11, 12, 13, 14, 15, 16, 17, 18, 19, 20, 21, 22, 23, and/or 24 biomarkers may be compared to each other and/or to a reference that is internal or external. It is contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, and/or 66 biomarkers may be compared to each other and/or to a reference that is internal or external. A person of ordinary skill in the art would know how to do such comparisons.

Comparisons or results from comparisons may reveal or be expressed as x-fold increase or decrease in expression relative to a standard or relative to another biomarker or relative to the same biomarker but in a different class of prognosis. In some embodiments, patients with a poor prognosis have a relatively high level of expression (overexpression) or relatively low level of expression (underexpression) when compared to patients with a better or favorable prognosis, or vice versa.

Fold increases or decreases may be, be at least, or be at most 1-, 2-, 3-, 4-, 5-, 6-, 7-8-, 9-, 10-, 11-, 12-, 13-, 14-, 15-, 16-, 17-, 18-, 19-, 20-, 25-, 30-, 35-, 40-, 45-, 50-, 55-, 60-65-, 70-, 75-, 80-, 85-, 90-, 95-, 100- or more, or any range derivable therein. Alternatively, differences in expression may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000% difference, or any range derivable therein.

Other ways to express relative expression levels are by normalized or relative numbers such as 0.001, 0.002, 0.003, 0.004, 0.005, 0.006, 0.007, 0.008, 0.009, 0.01, 0.02, 0.03. 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein.

In some embodiments, algorithms, such as the weighted voting programs, can be used to facilitate the evaluation of biomarker levels. In addition, in some embodiments, other clinical evidence can be combined with a biomarker-based test to reduce the risk of false evaluations. In some embodiments, other cytogenetic evaluations may be considered.

Any biological sample from the patient that contains breast cancer cells may be used to evaluate the expression pattern of any biomarker discussed herein. In some embodiments, a biological sample from a breast tumor is used. Evaluation of a biological sample may involve, though it need not involve, panning (enriching) for cancer cells or isolating the cancer cells.

### A. Nucleic Acids

Screening methods based on differentially expressed gene products are well known in the art. In accordance with one aspect of the present invention, the differential expression patterns of breast cancer biomarkers can be determined by measuring the levels of RNA transcripts of these genes, or genes whose expression is modulated by the these genes, in the patient's breast cancer cells. Suitable methods for this purpose include, but are not limited to, RNA sequencing, RT-PCR, Northern Blot, microarray, in situ hybridization, slot-blotting, nuclease protection assay, and oligonucleotide arrays.

In some embodiments, gene expression is determined from a biological sample obtained from neoplastic cells in fresh tumor biopsies, fixated tumor biopsies (e.g., those fixed and/or embedded using formalin, paraformaldehyde, paraffin, etc.), blood, tears, semen, saliva, urine, tissue, breast milk, lymph fluid, stool, sputum, cerebrospinal fluid, and supernatant from cell lysate.

In certain embodiments, gene expression is determined from a Formalin-Fixed Paraffin-Embedded (FFPE) biological sample.

In certain aspects, RNA isolated from cancer cells (e.g., breast cancer cells) can be amplified to cDNA or cRNA before detection and/or quantitation. In some embodiments, isolated RNA can be either total RNA or mRNA. In some embodiments, RNA amplification can be specific or non-specific. In some embodiments, suitable amplification methods for amplifying nucleic acid targets include, but are not limited to, RT-PCR, isothermal amplification, ligase chain reaction, and Qbeta replicase. In some embodiments, suitable hybridization-based amplification methods post target hybridization include, but are not limited to, branched DNA, hybridization chain reaction, rolling cycle amplification, and/or click chemistry-based amplification. In some embodiments, amplified nucleic acid products can be detected and/or quantitated through hybridization to labeled probes. In some embodiments, labeled probes can be further amplified with a general amplification method including, but not limited to, biotin-(strept)avidin, antibodies, and/or tyramide signal amplification. In some embodiments, a labeled probe is conjugated to a horseradish peroxidase molecule which reacts with a plurality of tyramide-fluorophore molecules, and/or a plurality of tyramide-hapten or tyramide-biotin molecules. In some embodiments, hapten molecules include, but are not limited to, biotin, digoxygenin, dinitrophenyl, trnitrophenyl, and/or a fluorophore. In some embodiments, labeled probes can be conjugated to a fluorophore for visualization (e.g., utilizing an epifluorescent microscope, etc.) and/or conjugated to one or more molecules suitable for a chromogenic reaction(s) (e.g., horseradish peroxidase, alkaline phosphatase, etc.) suitable for direct visualization (e.g., with compound light microscope, etc.). In some embodiments, detection may involve fluorescence resonance energy transfer (FRET) or some other kind of quantum dots.

In some embodiments, amplification primers or hybridization probes for a breast cancer biomarker can be prepared from the gene sequence or obtained through commercial sources, such as Affymetrix, NanoString, Illumina BeadChip, etc. In certain embodiments a gene sequence is identical or complementary to at least 8 contiguous nucleotides of the coding sequence.

In some embodiments, sequences suitable for making probes/primers for detection of their corresponding breast cancer biomarkers include those that are identical or complementary to all or part of genes (see e.g., Table 1) or SEQ ID NOs described herein. These sequences are all nucleic acid sequences of breast cancer biomarkers.

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In some embodiments, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene or SEQ ID NO described herein. Preferably, each probe/primer has relatively high sequence complexity and does not have any ambiguous residue (undetermined “n” residues). In some embodiments, probes/primers can hybridize to a target gene, including its RNA transcripts, such as mRNA transcripts and the like, under stringent or highly stringent conditions. In some embodiments, because each of the biomarkers has more than one human sequence, it is contemplated that probes and primers may be designed for use with each one of these sequences. For example, inosine is a nucleotide frequently used in probes or primers to hybridize to more than one sequence. It is contemplated that probes or primers may have inosine or other design implementations that accommodate recognition of more than one human sequence for a particular biomarker.

In some embodiments, a probe/primer targets an ACOX2 gene (Accession NM_003500.3). In some embodiments, an ACOX2 targeting probe/primer comprises or consists, or comprises or consists of a sequence complementary to, SEQ ID NO: 1.

(SEQ ID NO: 1)
ACTACCAGACACAACAGCAGAAACTCTTTCCTCAGCTGGCCATCAGTTA
TGCCTTCCATTTCCTGGCAGTCAGCCTCTTGGAGTTCTTCCAGCACTCC
TA.

In some embodiments, a probe/primer targets an ADCY1 gene (Accession NM_001281768.1). In some embodiments, an ADCY1 targeting probe/primer comprises or consists of, or comprises or consists of a sequence complementary to, SEQ ID NO: 2.

(SEQ ID NO: 2)
CCAGGGAAGGTTCATATCACAAAGACGACCCTAGCGTGCTTGAATGGGG
ACTACGAGGTAGAACCGGGTTACGGACATGAGAGGAACAGTTTCTTGAA
AA.

In some embodiments, a probe/primer targets a B2M gene (Accession NM_004048.2). In some embodiments, a probe/primer that targets B2M gene is a control (e.g., as a “housekeeping” control). In some embodiments, a B2M targeting probe/primer comprises or consists of, or comprises or consists of a sequence complementary to, SEQ ID NO: 3.

(SEQ ID NO: 3)
TACTGAAGAATGGAGAGAGAATTGAAAAAGTGGAGCATTCAGACTTGTC
TTTCAGCAAGGACTGGTCTTTCTATCTCTTGTACTACACTGAATTCACC
CC.

In some embodiments, a probe/primer targets a CALCR gene (Accession NM_001742.2). In some embodiments, a CALCR targeting probe/primer comprises or consists of, or comprises or consists of a sequence complementary to, SEQ ID NO: 4.

(SEQ ID NO: 4)
TTACATCTGCCATCAGGAGCTGAGGAATGAACCAGCCAACAACCAAGGC
GAGGAGAGTGCTGAGATCATCCCTTTGAATATCATAGAGCAAGAGTCAT
CT.

In some embodiments, a probe/primer targets a CHST8 gene (Accession NM_001127895.1). In some embodiments, a CHST8 targeting probe/primer comprises or consists of, or comprises or consists of a sequence complementary to, SEQ ID NO: 5.

(SEQ ID NO: 5)
AGTTTGAGCACCCCAACAGCTACTATCACCCGGTCTTCGGCAAGGCCAT
CCTGGCCCGGTACCGCGCCAATGCCTCTCGGGAGGCCCTGCGGACCGGC
TC.

In some embodiments, a probe/primer targets a COL3A1 gene (Accession NM_000090.3). In some embodiments, a COL3A1 targeting probe/primer comprises or consists of, or comprises or consists of a sequence complementary to, SEQ ID NO: 6.

(SEQ ID NO: 6)
TTGGCACAACAGGAAGCTGTTGAAGGAGGATGTTCCCATCTTGGTCAGT
CCTATGCGGATAGAGATGTCTGGAAGCCAGAACCATGCCAAATATGTGT
CT.

In some embodiments, a probe/primer targets a CXCL12 gene (Accession NM_199168.3). In some embodiments, a CXCL12 targeting probe/primer comprises or consists of, or comprises or consists of a sequence complementary to, SEQ ID NO: 7.

(SEQ ID NO: 7)
CCGCCCGCCCGCCCGCCCGCGCCATGAACGCCAAGGTCGTGGTCGTGCT
GGTCCTCGTGCTGACCGCGCTCTGCCTCAGCGACGGGAAGCCCGTCAGC
CT.

In some embodiments, a probe/primer targets an ELOVL2 gene (Accession NM_017770.3). In some embodiments, an ELOVL2 targeting probe/primer comprises or consists of, or comprises or consists of a sequence complementary to, SEQ ID NO: 8.

(SEQ ID NO: 8)
GTAACAAGTATATGAAGAACAGACCTGCTCTTTCTCTCAGGGGTATCCT
CACCTTGTATAATCTTGGAATCACACTTCTCTCCGCGTACATGCTGGCA
GA.

In some embodiments, a probe/primer targets a GAPDH gene (Accession NM_001256799.1). In some embodiments, a probe/primer that targets GAPDH gene is a control (e.g., as a “housekeeping” control). In some embodiments, a GAPDH targeting probe/primer comprises or consists of, or comprises or consists of a sequence complementary to, SEQ ID NO: 9.

(SEQ ID NO: 9)
GAACGGGAAGCTTGTCATCAATGGAAATCCCATCACCATCTTCCAGGAG
CGAGATCCCTCCAAAATCAAGTGGGGCGATGCTGGCGCTGAGTACGTCG
TG.

In some embodiments, a probe/primer targets a GFRA1 gene (Accession NM_005264.4). In some embodiments, a GFRA1 targeting probe/primer comprises or consists of, or comprises or consists of a sequence complementary to, SEQ ID NO: 10.

(SEQ ID NO: 10)
CCAGCAGGGTCTGAGAATGAAATTCCCACTCATGTTTTGCCACCGTGTG
CAAATTTACAGGCACAGAAGCTGAAATCCAATGTGTCGGGCAATACACA
CC.

In some embodiments, a probe/primer targets a GJA1 gene (Accession NM_000165.3). In some embodiments, a GJA1 targeting probe/primer comprises or consists of, or comprises or consists of a sequence complementary to, SEQ ID NO: 11.

(SEQ ID NO: 11)
GCGAACCTACATCATCAGTATCCTCTTCAAGTCTATCTTTGAGGTGGCC
TTCTTGCTGATCCAGTGGTACATCTATGGATTCAGCTTGAGTGCTGTTT
AC.

In some embodiments, a probe/primer targets a GREB1 gene (Accession NM_014668.3). In some embodiments, a GREB1 targeting probe/primer comprises or consists of, or comprises or consists of a sequence complementary to, SEQ ID NO: 12.

(SEQ ID NO: 12)
CAGCTTCAGTCACCTTTCCAGTGGTGGCCTCTGGAGAACCAGTGTCTGT
TCCTGACAACTTGCTGAAAATATGCAAGGCCAAGCCAGTGATATTTAAA
GG.

In some embodiments, a probe/primer targets a KRT13 gene (Accession NM_002274.3). In some embodiments, a KRT13 targeting probe/primer comprises or consists of, or comprises or consists of a sequence complementary to, SEQ ID NO: 13.

(SEQ ID NO: 13)
TCTCCTGATGGTGGGCCCTCTGTGCTCTTCTCTTCCGGTCGGATCTCTC
TCCTCTCTGACCTGGATACGCTTTGGTTTCTCAACTTCTCTACCCCAAA
GA.

In some embodiments, a probe/primer targets a MAPT gene (Accession NM_001123066.2). In some embodiments, a MAPT targeting probe/primer comprises or consists of, or comprises or consists of a sequence complementary to, SEQ ID NO: 14.

(SEQ ID NO: 14)
ACTGCCAACAGTTTCGGCTGCATTTCTTCACGCACCTCGGTTCCTCTTC
CTGAAGTTCTTGTGCCCTGCTCTTCAGCACCATGGGCCTTCTTATACGG
AA.

In some embodiments, a probe/primer targets a MPPED2 gene (Accession NM_001584.2). In some embodiments, a MPPED2 targeting probe/primer comprises or consists of, or comprises or consists of a sequence complementary to, SEQ ID NO: 15.

(SEQ ID NO: 15)
TGAAACCAGAGGACTTTGACAATGTTCAGTCCCTCCTGACAAACAGTAT
TTACTTACAAGATTCGGAGGTAACAGTGAAGGGATTCAGGATATACGGT
GC.

In some embodiments, a probe/primer targets a MYB gene (Accession NM_001130173.1). In some embodiments, a MYB targeting probe/primer comprises or consists of, or comprises or consists of a sequence complementary to, SEQ ID NO: 16.

(SEQ ID NO: 16)
CCGCGCCCGCCGCGCCATGGCCCGAAGACCCCGGCACAGCATATATAGC
AGTGACGAGGATGATGAGGACTTTGAGATGTGTGACCATGACTATGATG
GG.

In some embodiments, a probe/primer targets a NPY1R gene (Accession NM_000909.4). In some embodiments, a NPY1R targeting probe/primer comprises or consists of, or comprises or consists of a sequence complementary to, SEQ ID NO: 17.

(SEQ ID NO: 17)
ACATACTTCTCAGCTGCAAATATTATGGAGAATTGGGGCACCCACAGGA
ATGAAGAGAGAAAGCAGCTCCCTAACTTCAAAACCATTTTGGTACCTGA
CA.

In some embodiments, a probe/primer targets a OLFM1 gene (Accession NM_006334.3). In some embodiments, an OLFM1 targeting probe/primer comprises or consists of, or comprises or consists of a sequence complementary to, SEQ ID NO: 18.

(SEQ ID NO: 18)
CACAGCAGACCATGTGTTCACGGGATGCCCGCACAAAACAGCTGAGGCA
GCTACTGGAGAAGGTGCAGAACATGTCTCAATCCATAGAGGTCTTGGAC
AG.

In some embodiments, a probe/primer targets a PDZK1 gene (Accession NM_002614.4). In some embodiments, a PDZK1 targeting probe/primer comprises or consists of, or comprises or consists of a sequence complementary to, SEQ ID NO: 19.

(SEQ ID NO: 19)
ACCGAGGGCCACCTGGTCCGGGTGGTTGAGAAGTGTAGCCCAGCAGAGA
AGGCTGGCCTTCAAGATGGAGACAGAGTTCTTAGGATCAATGGTGTCTT
TG.

In some embodiments, a probe/primer targets a PGR gene (Accession NM_000926.4). In some embodiments, a PGR targeting probe/primer comprises or consists of, or comprises or consists of a sequence complementary to, SEQ ID NO: 20.

(SEQ ID NO: 20)
AGCCAGCCAGAGCCCACAATACAGCTTCGAGTCATTACCTCAGAAGATT
TGTTTAATCTGTGGGGATGAAGCATCAGGCTGTCATTATGGTGTCCTTA
CC.

In some embodiments, a probe/primer targets a PSMC4 gene (Accession NM_006503.2). In some embodiments, a probe/primer that targets PSMC4 gene is a control (e.g., as a “housekeeping” control). In some embodiments, a PSMC4 targeting probe/primer comprises or consists of, or comprises or consists of a sequence complementary to, SEQ ID NO: 21.

(SEQ ID NO: 21)
CATCGGACAATTTCTGGAGGCTGTGGATCAGAATACAGCCATCGTGGGC
TCTACCACAGGCTCCAACTATTATGTGCGCATCCTGAGCACCATCGATC
GG.

In some embodiments, a probe/primer targets a PUM1 gene (Accession NM_001020658.1). In some embodiments, a probe/primer that targets PUM1 gene is a control (e.g., as a “housekeeping” control). In some embodiments, a PUM1 targeting probe/primer comprises or consists of, or comprises or consists of a sequence complementary to, SEQ ID NO: 22.

(SEQ ID NO: 22)
CTGGGGAACATCAGATCATTCAGTTTCCCAGCCAATCATGGTGCAGAGA
AGACCTGGTCAGAGTTTCCATGTGAACAGTGAGGTCAATTCTGTACTGT
CC.

In some embodiments, a probe/primer targets a RASGRP1 gene (Accession NM_005739.3). In some embodiments, a RASGRP1 targeting probe/primer comprises or consists of, or comprises or consists of a sequence complementary to, SEQ ID NO: 23.

(SEQ ID NO: 23)
ACGATCTCATTGACAGCTGCATTCAATCTTTTGATGCAGATGGAAACCT
GTGTCGAAGTAACCAACTGTTGCAAGTCATGCTGACCATGCACCGAATT
GT.

In some embodiments, a probe/primer targets a SGK1 gene (Accession NM_005627.2). In some embodiments, a SGK1 targeting probe/primer comprises or consists of, or comprises or consists of a sequence complementary to, SEQ ID NO: 24.

(SEQ ID NO: 24)
GTGTGAACCGTCGTGTGAGTGTGGTATGCCTGATCACAGATGGATTTTG
TTATAAGCATCAATGTGACACTTGCAGGACACTACAACGTGGGACATTG
TT.

In some embodiments, a probe/primer targets a STC2 gene (Accession NM_003714.2). In some embodiments, a STC2 targeting probe/primer comprises or consists of, or comprises or consists of a sequence complementary to, SEQ ID NO: 25.

(SEQ ID NO: 25)
ATTTCTATGTGTAATTTCTGAGCCATTGTACTGTCTGGGCTGGGGGGGA
CACTGTCCAAGGGAGTGGCCCCTATGAGTTTATATTTTAACCACTGCTT
CA.

In some embodiments, a probe/primer targets a TFF1 gene (Accession NM_003225.2). In some embodiments, a TFF1 targeting probe/primer comprises or consists of, or comprises or consists of a sequence complementary to, SEQ ID NO: 26.

(SEQ ID NO: 26)
TTTCGACGACACCGTTCGTGGGGTCCCCTGGTGCTTCTATCCTAATACC
ATCGACGTCCCTCCAGAAGAGGAGTGTGAATTTTAGACACTTCTGCAGG
GA.

In some embodiments, a probe/primer targets a TGM2 gene (Accession NM_004613.2). In some embodiments, a TGM2 targeting probe/primer comprises or consists of, or comprises or consists of a sequence complementary to, SEQ ID NO: 27.

(SEQ ID NO: 27)
AGGGCTTTATCTACCAGGGCTCGGCCAAGTTCATCAAGAACATACCTTG
GAATTTTGGGCAGTTTGAAGATGGGATCCTAGACATCTGCCTGATCCTT
CT.

In some embodiments, a probe/primer targets a VCAN gene (Accession NM_004385.3). In some embodiments, a VCAN targeting probe/primer comprises or consists of, or comprises or consists of a sequence complementary to, SEQ ID NO: 28.

(SEQ ID NO: 28)
GCAGGGTGCCCATCTCACAAGCATCCTGTCTCACGAAGAACAAATGTTT
GTTAATCGTGTGGGCCATGATTATCAGTGGATAGGCCTCAATGACAAGA
TG.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In some embodiments, probes/primers for a gene are selected from regions which significantly diverge from the sequences of other genes. Such regions can be determined by checking the probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length (W) in the query sequence, which either match or satisfy some positive-valued threshold score (T) when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by one of ordinary skill in the art.

In some embodiments, RT-qPCR (using an internal probe, such as TaqMan, ABI, etc., or without an internal probe e.g., SYBR green, etc.) is used for detecting and comparing the levels of RNA transcripts (e.g., mRNA transcripts) in cancer samples (e.g., breast cancer samples. RT-qPCR involves reverse transcription (RT) of RNA (e.g., mRNA) to cDNA followed by relative quantitative PCR (qPCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products preferably are carried out when the PCR reactions are in the linear portion of their curves. In addition, relative concentrations of the amplifiable cDNAs preferably are normalized to some independent standard, which may be based on either internally existing RNA species or externally introduced RNA species. The abundance of a particular mRNA species may also be determined relative to the average abundance of all mRNA species in the sample.

In some embodiments, PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in clinical samples is that they are of variable quantity and/or quality. This problem can be overcome if the RT-PCR is performed as a relative RT-qPCR with an internal standard in which the internal standard is an amplifiable cDNA fragment that is similar or larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative RT-qPCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs.

Nucleic acid arrays can also be used to detect and compare the differential expression patterns of breast cancer biomarkers in breast cancer cells. The probes suitable for detecting the corresponding breast cancer biomarkers can be stably attached to known discrete regions on a solid substrate. As used herein, a probe is “stably attached” to a discrete region if the probe maintains its position relative to the discrete region during the hybridization and the subsequent washes. Construction of nucleic acid arrays is well known in the art. Suitable substrates for making polynucleotide arrays include, but are not limited to, membranes, films, plastics and quartz wafers.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be 50, 100, 200, 300, 400, 500 or more probes/cm2.

Specifically contemplated by the present inventors are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more breast cancer biomarkers with respect to diagnostic, prognostic, and treatment methods of the disclosure.

The present disclosure may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO 0138580; WO 0168255; WO 03020898; WO 03040410; WO 03053586; WO 03087297; WO 03091426; WO 03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm2. The surface area of the array can be about or less than about 1, 1.6, 2, 3, 4, 5, 6, 7, 8, 9, or 10 cm2.

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448, all of which are specifically incorporated by reference.

In some embodiments, nuclease protection assays are used to quantify RNAs (e.g., mRNAs) derived from the breast cancer samples. There are many different versions of nuclease protection assays known to those practiced in the art. The common characteristic that these nuclease protection assays have is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

In some embodiments, gene expression is determined from a biological sample using 3′ RNA sequencing, using products such as Lexogen QuantSeq, QioSeq UPX 3′ Transcriptome, etc. In some embodiments, 3′ RNA sequencing does not require mRNA samples to be fragmented before reverse transcription, and cDNAs are reverse transcribed only from the 3′ RNA sequencing end of the mRNAs, resulting in only one copy of cDNA for each transcript, resulting in a direct 1:1 ratio between RNA and cDNA copy numbers.

In some embodiments, gene expression is determined from a biological sample using specific targeted sequencing, using products such as BioSpyder Temp0-Seq, Ion Ampliseq Transcriptome, etc. In some embodiments, specific targeted sequencing targets RNA (e.g., mRNA) sequences by hybridization to DNA oligos followed by removal of unhybridized oligos and amplification of remaining products.

### B. Proteins and Polypeptides

In other embodiments, the differential expression patterns of breast cancer biomarkers can be determined by measuring the levels of polypeptides encoded by these genes in breast cancer cells. Methods suitable for this purpose include, but are not limited to, immunoassays such as ELISA, RIA, FACS, dot blot, immunoblotting, immunohistochemistry, and antibody-based radioimaging. Protocols for carrying out these immunoassays are well known in the art. Other methods such as 2-dimensional SDS-polyacrylamide gel electrophoresis can also be used. These procedures may be used to recognize any of the polypeptides encoded by the breast cancer biomarker genes described herein.

One example of a method suitable for detecting the levels of target proteins in biological samples is ELISA. In an exemplifying ELISA, antibodies capable of binding to the target proteins encoded by one or more breast cancer biomarker genes are immobilized onto a selected surface exhibiting protein affinity, such as wells in a polystyrene or polyvinylchloride microtiter plate. Then, breast cancer cell samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Before being added to the microtiter plate, cells in the peripheral blood samples can be lysed using various methods known in the art. Proper extraction procedures can be used to separate the target proteins from potentially interfering substances.

In another ELISA embodiment, the breast cancer cell samples containing the target proteins are immobilized onto the well surface and then contacted with antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another typical ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control and/or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex then requires a labeled secondary binding ligand or antibody, or a secondary binding ligand or antibody in conjunction with a labeled tertiary antibody or third binding ligand.

After all of the incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In some embodiments, a label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl)-benzhiazoline-6-sulfonic acid (ABTS) and hydrogen peroxide, in the case of peroxidase as the enzyme label. Quantitation can be achieved by measuring the degree of color generation, e.g., using a spectrophotometer.

In some embodiments, another suitable method is RIA (radioimmunoassay). An example of RIA is based on the competition between radiolabeled-polypeptides and unlabeled polypeptides for binding to a limited quantity of antibodies. Suitable radiolabels include, but are not limited to, I125. In some embodiments, a fixed concentration of I125-labeled polypeptide is incubated with a series of dilution of an antibody specific to the polypeptide. When the unlabeled polypeptide is added to the system, the amount of the I125-polypeptide that binds to the antibody is decreased. A standard curve can therefore be constructed to represent the amount of antibody-bound I125-polypeptide as a function of the concentration of the unlabeled polypeptide. From this standard curve, the concentration of the polypeptide in unknown samples can be determined. Various protocols for conducting RIA to measure the levels of polypeptides in breast cancer cell samples are well known in the art.

In some embodiments, suitable antibodies for biomarker detection include, but are not limited to, polyclonal antibodies, monoclonal antibodies, chimeric antibodies, humanized antibodies, single chain antibodies, Fab fragments, and fragments produced by a Fab expression library.

In some embodiments, antibodies can be labeled with one or more detectable moieties to allow for detection of antibody-antigen complexes. In some embodiments, detectable moieties can include compositions detectable by spectroscopic, enzymatic, photochemical, biochemical, bioelectronic, immunochemical, electrical, optical or chemical means. In some embodiments, detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Protein array technology is discussed in detail in Pandey and Mann (2000) and MacBeath and Schreiber (2000), each of which is herein specifically incorporated by reference. These arrays typically contain thousands of different proteins or antibodies spotted onto glass slides or immobilized in tiny wells and allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as a breast cancer biomarker proteins.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

In some embodiments, the levels of polypeptides in a biological sample can be determined by detecting the biological activities associated with the polypeptides. If a biological function/activity of a polypeptide is known, suitable in vitro bioassays can be designed to evaluate the biological function/activity, thereby determining the amount of the polypeptide in the sample. In some embodiments, levels of polypeptides and/or biological activities associated with the polypeptides can be determined using assays comprising flow cytometry.

In some embodiments, the levels of polypeptides in a biological sample can be determined by mass spectrometry, including but not limited to methods such as SILAC, TMT labeling, and immunoprecipitation (IP)-MS/MS.

### IV. Breast Cancer Therapy

In some embodiments, methods described herein are not limited to breast cancer, but are applicable to other ER+ cancers, such as ovarian and/or endometrial cancer (e.g., serous carcinoma, mucinous carcinoma, endometrioid carcinoma, clear cell carcinoma, etc.), or secondary tumors derived from metastatic breast, ovarian, and/or endometrial cancers. In some embodiments, methods described herein are suitable for ER+ ovarian cancer.

Certain embodiments are directed to methods of treating breast cancer based on responsiveness to ER antagonism of breast cancer tissue.

In certain aspects, there may be provided methods for treating a subject determined to have cancer and with a predetermined expression profile of one or more biomarkers disclosed herein.

In certain embodiments, methods are directed to treating breast cancer patients who have metastatic ER+ breast cancer but have failed and/or are failing endocrine therapy. In certain embodiments, a patient sample is taken during endocrine therapy administration, and is a Formalin-Fixed Paraffin-Embedded (FFPE) sample.

Therapy provided herein may comprise administration of a combination of therapeutic agents, such as for example, a first cancer therapy (e.g., radiotherapy) and a second cancer therapy (e.g., ET). The therapies may be administered in any suitable manner known in the art. For example, the first and second cancer treatment may be administered sequentially (at different times) or concurrently (at the same time).

In some aspects, the first cancer therapy and the second cancer therapy are administered substantially simultaneously. In some aspects, the first cancer therapy and the second cancer therapy are administered sequentially. In some aspects, the first cancer therapy, the second cancer therapy, and a third therapy are administered sequentially. In some aspects, the first cancer therapy is administered before administering the second cancer therapy. In some aspects, the first cancer therapy is administered after administering the second cancer therapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

Therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose (also “effective amount” or “therapeutically effective amount”) is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120,125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 10 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the un-metabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

In a further aspect, biomarkers and related systems that can establish a prognosis of cancer patients with respect to ER antagonist/inhibitor therapy can be used to identify patients who may get benefit of conventional single or combined modality therapy.

In certain aspects, conventional cancer therapy may be applied to a subject wherein the subject is identified or reported as likely responsive to an ER antagonist/inhibitor based on the assessment of the biomarkers as disclosed. On the other hand, at least an alternative cancer therapy may be prescribed, as used alone or in combination with conventional cancer therapy, if a poor prognosis is determined by the disclosed methods, systems, or kits.

Certain embodiments concern an ER antagonist. In some embodiments, an ER antagonist is a selective estrogen receptor antagonist. In some embodiments, an ER antagonist is a non-selective estrogen receptor antagonist. In some embodiments, an ER antagonist is steroidal. In some embodiments, an ER antagonist is nonsteroidal. It is specifically contemplated that one or more of the antagonists discussed herein or in the incorporated references may be excluded in embodiments of the invention. It is also contemplated that in some embodiments, more than one ER antagonist is employed, while in other embodiments, only one is employed as part of the therapeutic method (though it may be administered multiple times), while in still other embodiments no ER antagonists are employed. It is contemplated that the second one may be administered concurrently with the first one or they may be administered at different times.

Conventional cancer therapies include one or more selected from the group of chemical or radiation based treatments and surgery. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), taxol, gemcitabine, navelbine, farnesyl-protein transferase inhibitors, transplatinum, capecitabine, vincristin, vinblastin and methotrexate, or any analog or derivative variant of the foregoing. A subset of these are ET and include but are not limited to, e.g., tamoxifen, raloxifene, fulvestrant, and other ER binding agents.

Suitable therapeutic agents include, for example, vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), serine kinase targeting agents, transitional metal complexes, proteasome inhibitors, antimetabolites (such as nucleoside analogs), alkylating agents, platinum-based agents, anthracycline antibiotics, topoisomerase inhibitors, macrolides, therapeutic antibodies, retinoids (such as all-trans retinoic acids or a derivatives thereof); geldanamycin or a derivative thereof (such as 17-AAG), and other standard chemotherapeutic agents well recognized in the art.

Certain chemotherapeutics are well known for use against breast cancer. In some embodiments, these breast cancer chemotherapeutics include for example, capecitabine, carboplatin, cyclophosphamide (Cytoxan), daunorubicin, docetaxel (Taxotere), doxorubicin (Adriamycin), epirubicin (Ellence), fluorouracil (also called 5-fluorouracil or 5-FU), gemcitabine, eribulin, ixabepilone, methotrexate, mitomycin C, mitoxantrone, paclitaxel (Taxol), thiotepa, vincristine, and vinorelbine.

In some embodiments, a chemotherapeutic agent is any of (and in some embodiments selected from the group consisting of) adriamycin, colchicine, cyclophosphamide, actinomycin, bleomycin, daunorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Herceptin®, vinorelbine, Doxil®, capecitabine, Gleevec®, Alimta®, Avastin®, Velcade®, Tarceva®, Neulasta®, Lapatinib, STI-571, ZD1839, Iressa® (gefitinib), SH268, genistein, CEP2563, SU6668, SU11248, EMD121974, and Sorafenib.

In some embodiments, a chemotherapeutic agent is a composition comprising nanoparticles comprising a thiocolchicine derivative and a carrier protein (such as albumin).

In some embodiments a combination of chemotherapeutic agents is administered to breast cancer cells. In some embodiments, chemotherapeutic agents may be administered serially (within minutes, hours, or days of each other) or in parallel; they also may be administered to the patient in a pre-mixed single composition. In some embodiments, composition may or may not contain an estrogen receptor antagonist.

In some embodiments, systemic therapy regimens suitable for treatment of ER+ cancer include, but are not limited to: anthracyclines (e.g., doxorubicin or liposomal doxorubicin); taxanes (e.g., paclitaxel); anti-metabolites (e.g., capecitabine or gemcitabine); microtubule inhibitors (e.g., vinorelbine or eribulin); for BRCA1 or BRCA2 mutations olaparib, talazoparib, platinum, carboplatin, and/or cisplatin; for NTRK fusion, larotrectinib or entrectinib; for MSI-H/dMMR, pembrolizumab; cyclophosphamide; docetaxel; albumin-bound paclitaxel; epirubicin; ixabepilone; AC (doxorubicin with cyclophosphamide); EC (epirubicin with cyclophosphamide); CMF (cyclophosphamide with methotrexate and fluorouracil); docetaxel with capecitabine; GT (gemcitabine with paclitaxel); gemcitabine with carboplatin; paclitaxel with bevacizumab; and/or carboplatin with paclitaxel or albumin-bound paclitaxel.

In some embodiments, ER+ cancer therapeutic regimens include, but are not limited to: endocrine therapies, such as a SERD (e.g., fulvestrant); aromatase inhibitor with CDK4/6 inhibitor (e.g., abemaciclib, palbociclib, or ribociclib); SERD (e.g., fulvestrant) with or without a non-steroidal aromatase inhibitor (e.g., anastrozole, letrozole); fulvestrant with CDK4/6 inhibitor (e.g., abemaciclib, palbociclib, or ribociclib); non-steroidal aromatase inhibitor (e.g., anastrozole, letrozole); SERM (e.g., tamoxifen or toremifene); steroidal aromatase inactivator (e.g., exemestane); if PIK3CA is mutated, a PI3Kα-specific inhibitor (e.g., alpelisib) and fulvestrant; mammalian target of rapamycin (mTOR) inhibitor (e.g., everolimus) with endocrine therapy (e.g., exemestane, fulvestrant, tamoxifen); megestrol acetate; fluoxymesterone; ethinylestradiol; abemaciclib; for BRCA1 or BRCA2 mutations, poly(ADP-ribose) polymerase (PARP) inhibitors (e.g., olaparib or talazoparib); for NTRK fusion, larotrectinib or entrectinib, for MSI-H/dMMR, pembrolizumab; and/or a CDK4/6 inhibitor (e.g., abemaciclib, palbociclib, or ribociclib) without a combinatorial ET.

Various combinations with an ER antagonist and an anticancer agent or compound (or a combination of such agents and/or compounds) may be employed, for example ER antagonist is “A” and an anticancer agent or compound (or a combination of such agents and/or compounds) given as part of an anticancer therapy regimen, is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of a therapeutic compounds or agents to a patient will follow general protocols for the administration of such compounds, taking into account the toxicity, if any, of a therapy. It is expected that treatment cycles would be repeated as necessary. It also is contemplated that various standard therapies, as well as surgical intervention, may be applied in combination with a described therapy.

The term “a serine/threonine kinase inhibitor”, as used herein, relates to a compound which inhibits serine/threonine kinases. An example of a target of a serine/threonine kinase inhibitor includes, but is not limited to, dsRNA-dependent protein kinase (PKR). Examples of indirect targets of a serine/threonine kinase inhibitor include, but are not limited to, MCP-1, NF-kappaB, eIF2alpha, COX2, RANTES, IL8, CYP2A5, IGF-1, CYP2B1, CYP2B2, CYP2H1, ALAS-1, HIF-1, erythropoietin and/or CYP1A1. An example of a serine/threonine kinase inhibitor includes, but is not limited to, Sorafenib and 2-aminopurine, also known as 1H-purin-2-amine(9CI). Sorafenib is marketed as NEXAVAR.

Other examples of anticancer therapy that may be used in conjunction with ER antagonist therapy include but are not limited to checkpoint inhibitors such as those that inhibit PD-1 (e.g., Pembrolizumab and Nivolumab), PD-L1 (e.g., Atezolizumab, Avelumab, Durvalumab), or CTLA-4 (e.g., Ipilimumab).

The term “an angiogenesis inhibitor”, as used herein, relates to a compound which targets, decreases or inhibits the production of new blood vessels. Targets of an angiogenesis inhibitor include, but are not limited to, methionine aminopeptidase-2 (MetAP-2), macrophage inflammatory protein-1 (MIP-la), CCL5, TGF-beta, lipoxygenase, cyclooxygenase, and topoisomerase. Indirect targets of an angiogenesis inhibitor include, but are not limited to, p21, p53, CDK2, and collagen synthesis. Examples of an angiogenesis inhibitor include, but are not limited to, Fumagillin, which is known as 2,4,6,8-decatetraenedioic acid, mono[3R,4S,5S,6R)-5-methoxy-4-[(2R,3R)-2-methyl-3-(3-methyl-2-butenyl)oxi-ranyl]-1-oxaspiro[2.5]oct-6-yl]ester, (2E,4E,6E,8E)-(9CI); Shikonin, which is also known as 1,4-naphthalenedione, 5,8-dihydroxy-2-[(1R)-1-hydroxy-4-methyl-3-pentenyl]-(9CI); Tranilast, which is also known as benzoic acid, 2-[[3-(3,4-dimethoxyphenyl)-1-oxo-2-propenyl]amino]-(9CI); ursolic acid; suramin; thalidomide and lenalidomide, and marketed as REVLIMID.

Radiation therapy that cause DNA damage and have been used extensively include what are commonly known as 7-rays, X-rays, and/or the directed delivery of radioisotopes to tumor cells. Proton beam therapy or proton therapy is frequently used for cancer treatment. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In some aspects, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A non-limiting example of ionizing radiation is x-radiation. Means for delivering x-radiation to a target tissue or cell are well known in the art.

In some aspects, the radiotherapy can comprise external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of radiotherapy administered to a subject may be presented as a total dose of radiotherapy, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy, and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, pre-cancers, or incidental amounts of normal tissue.

Laser therapy is the use of high-intensity light to destroy tumor cells. Laser therapy affects the cells only in the treated area. Laser therapy may be used to destroy cancerous tissue and relieve a blockage in the esophagus when the cancer cannot be removed by surgery. The relief of a blockage can help to reduce symptoms, especially swallowing problems.

Photodynamic therapy (PDT), a type of laser therapy, involves the use of drugs that are absorbed by cancer cells; when exposed to a special light, the drugs become active and destroy the cancer cells. PDT may be used to relieve symptoms of esophageal cancer such as difficulty swallowing.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well. A patient may be administered a single compound or a combination of compounds described herein in an amount that is, is at least, or is at most 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg (or any range derivable therein). A patient may be administered a single compound or a combination of compounds described herein in an amount that is, is at least, or is at most 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500 mg/kg/day (or any range derivable therein).

Alternative cancer therapy include any cancer therapy other than surgery, chemotherapy and radiation therapy in the present invention, such as immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

For example, the alternative cancer therapy may be a targeted therapy. The targeted therapy may be an anti-EGFR treatment. In some embodiments an anti-EGFR agent used is a tyrosine kinase inhibitor. Examples of suitable tyrosine kinase inhibitors are the quinazoline derivatives described in WO 96/33980, in particular gefitinib (Iressa). Other examples include quinazoline derivatives described in WO 96/30347, in particular erlotinib (Tarceva), dual EGFR/HER2 tyrosine kinase inhibitors, such as lapatinib, or pan-Erb inhibitors. In a preferred embodiment of the method or use of the invention, the anti-EGFR agent is an antibody capable of binding to EGFR, i.e. an anti-EGFR antibody.

In a further embodiment, the anti-EGFR antibody is an intact antibody, i.e. a full-length antibody rather than a fragment. An anti-EGFR antibody used in the method of the present invention may have any suitable affinity and/or avidity for one or more epitopes contained at least partially in EGFR. Preferably, the antibody used binds to human EGFR with an equilibrium dissociation constant (KD) of 10-8 M or less, more preferably 10-10 M or less.

In some embodiments, particularly antibodies for use include zalutumumab (2F8,), cetuximab (Erbitux), nimotuzumab (h-R3), panitumumab (ABX-EGF), and matuzumab (EMD72000), or a variant antibody of any of these, or an antibody which is able to compete with any of these, such as an antibody recognizing the same epitope as any of these. Competition may be determined by any suitable technique. In some embodiments, competition is determined by an ELISA assay. Often competition is marked by a significantly greater relative inhibition than 5% as determined by ELISA analysis.

Immunotherapeutics, generally, rely on the use of immune effector cells and molecules to target and destroy cancer cells. An immune effector may be, for example, an antibody specific for some marker on the surface of a tumor cell. An antibody alone may serve as an effector of therapy or it may recruit other cells to actually effect cell killing. An antibody also may be conjugated to a drug or toxin (chemotherapeutic, radionuclide, ricin A chain, cholera toxin, pertussis toxin, etc.) and serve merely as a targeting agent. Alternatively, an effector may be a lymphocyte carrying a surface molecule that interacts, either directly or indirectly, with a tumor cell target. Various effector cells include cytotoxic T cells and NK cells.

Gene therapy is the insertion of polynucleotides, including DNA or RNA, into an individual's cells and tissues to treat a disease. Antisense therapy is also a form of gene therapy in the present invention. A therapeutic polynucleotide may be administered before, after, or at the same time of a first cancer therapy. Delivery of a vector encoding a variety of proteins is encompassed within the invention. For example, cellular expression of exogenous tumor suppressor oncogenes would exert their function to inhibit excessive cellular proliferation, such as p53, p16, and C-CAM.

In some embodiments, additional agents can be used to improve therapeutic efficacy of treatment. In some embodiments, additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. In some embodiments, immunomodulatory agents include but are not limited to, tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that upregulation of cell surface receptors or their ligands such as Fas/Fas ligand, DR4 or DR5/TRAIL would potentiate apoptotic inducing abilities establishment of an autocrine or paracrine effect on hyperproliferative cells. In some embodiments, increases of intercellular signaling by elevating the number of GAP junctions would increase anti-hyperproliferative effects on the neighboring hyperproliferative cell population. In some embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. In some embodiments, it is further contemplated that other agents that increase the sensitivity of a hyperproliferative cell to apoptosis, such as the antibody c225, could be used in combination with the present invention to improve the treatment efficacy.

In some embodiments, hormonal therapy may be recommended alone or in combination with any other cancer therapy previously described. Use of hormones may be employed in treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

In some embodiments, ovarian ablation or suppression is recommended alone or in combination with any other cancer therapy described herein.

In some embodiments, technologies provided herein identify ESR1 fusion protein activity. In some embodiments, technologies provided herein identify ESR1 point mutations and/or ESR1 translocations. In some embodiments, technologies provided herein identify functionality of an ESR1 gene and associated ESR1 protein product. In some embodiments, gene expression profiling techniques described herein can be used to diagnose ESR1 point mutations and/or ESR1 translocations.

In some embodiments, evaluation of biomarkers as described herein may occur following a patient's exposure to adjuvant ET or in patients with metastatic breast cancer previously treated with non-combinatorial ET. In some embodiments, a biological sample (e.g., a tumor biopsy) is taken while a patient is receiving an ET. In some embodiments, a biological sample is taken after a patient has stopped receiving an ET.

In some embodiments, evaluation of biomarkers as described herein facilitates determination of ESR1 mutation status. In some embodiments, a patient's biological sample will return a positive score indicative of an ESR1 mutation, in such conditions an ET, e.g., a SERD etc. (e.g., preferably one that has not been utilized previously) with or without a CDK4/6 inhibitor can be a preferred therapeutic regimen. In some embodiments, a patient's biological sample will return a positive score indicative of a mutated ESR1 loci, in such conditions a reflexive diagnostic technique (e.g., a follow-up/secondary diagnostic technique) such as RNA sequencing can be utilized to determine if the mutated ESR1 represents a fusion. In some embodiments, when a mutated ESR1 is a fusion, no further ET is prescribed, and a CDK4/6 inhibitor monotherapy (e.g., abemaciclib) can be a preferred therapeutic regimen. In some embodiments, a patient's biological sample will return a positive score indicative of ESR1 protein activity, but no ESR1 fusion or mutant is identified, in such conditions an ET, e.g., a SERD etc. (e.g., preferably one that has not been utilized previously) with or without CDK4/6 inhibitor can be a preferred therapeutic regimen. In some embodiments, a patient's biological sample will return a negative score indicative of a WT ESR1 locus, in such conditions an ET, e.g., a SERD etc. (e.g., preferably one that has not been utilized previously) with or without CDK4/6 inhibitor can be used as a preferred therapeutic regimen.

### V. Kits

In some embodiments, the present invention also concern kits containing compositions of the disclosure or compositions to implement methods of the disclosure. In some aspects, kits can be used to evaluate one or more biomarkers (e.g., as described herein). In some aspects, kits can be used to detect, for example, genomic loss, reduced expression, or increased expression of a gene (e.g., those described herein). In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 100, 132, 500, 1,000 or more probes, primers or primer sets, synthetic molecules or inhibitors, or any value or range and combination derivable therein.

In certain embodiments, a kit comprises one or more probes/primers comprising or consisting of, or comprising or consisting of a sequence complementary to, SEQ ID NO: 1 to SEQ ID NO: 28. In certain embodiments, a kit comprises 1, 2, 3, 4, 5, 6, 7, 8, 9, or 10 probes/primers targeting a reference control gene. In some embodiments, a reference control gene may be B2M, GAPDH, PSMC4, and/or PUM1.

In some embodiments, a kit can be prepared from readily available materials and reagents. For example, such kits can comprise any one or more of the following materials: enzymes, reaction tubes, buffers, detergent, primers, probes, antibodies. In some embodiments, a kit allows a practitioner to obtain samples of neoplastic cells in fresh tumor biopsies, fixated tumor biopsies (e.g., those fixed and/or embedded using formalin, paraformaldehyde, paraffin, etc.), blood, tears, semen, saliva, urine, tissue, serum, breast milk, lymph fluid, stool, sputum, cerebrospinal fluid, and supernatant from cell lysate. In another preferred embodiment these kits include the needed apparatus for performing RNA extraction, RT-PCR, oligonucleotide quantification, and/or gel electrophoresis. Instructions for performing associated assays can also be included in a kit.

In some embodiments, a kit may comprise a number of agents for assessing differential expression of a number of biomarkers, for example, at least one of ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

In some embodiments, a kit may comprise a number of agents for assessing differential expression of a plurality of biomarkers, for example, at least one of CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR, KRT13, VCAN, COL3A1, CXCL12, GJA1, and TGM2.

In some embodiments, a kit may comprise a number of agents for assessing differential expression of a plurality of biomarkers, for example, at least one of ADCY1, GREB1, MYB, NPY1R, PGR, and TFF1.

In some embodiments, a kit may comprise reagents for detection of 1, 2, 3, 4, 5, 6, 7, 8, 9, 10, 11, 12, 13, 14, 15, 16, 17, 18, 19, 20, 21, 22, 23, and/or 24 biomarkers. In some embodiments, a kit may comprise reagents for detection of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, and/or 66 biomarkers.

In some embodiments, a kit is housed in a container. Kits may further comprise instructions for using the kit for assessing expression, means for converting the expression data into expression values and/or means for analyzing expression values to generate prognosis. Agents in a kit for measuring biomarker expression may comprise a plurality of PCR probes and/or primers for qRT-PCR and/or a plurality of antibody or fragments thereof for assessing expression of biomarkers. In another embodiment, agents in a kit for measuring biomarker expression may comprise an array of polynucleotides complementary to mRNAs of biomarkers identified herein. Possible means for converting expression data into expression values and for analyzing expression values to generate scores that predict survival or prognosis may be also included.

In certain embodiments, a kit is capable of detecting one or more target nucleic acids in situ. In some embodiments, a kit comprises sets of paired target probes capable of hybridizing to target nucleic acids and reagents necessary for a hybridization-based amplification method. In some embodiments, the hybridization-based amplification method can comprise one or more of, but is not limited to, branched DNA-based amplification and/or hybridization chain reaction. In some embodiments utilizing branched DNA amplification, a kit comprises preamplifier and amplifier oligonucleotides capable of hybridizing to each set of target probes, and a set of label probes capable of hybridizing to the preamplifiers. In some embodiments utilizing hybridization chain reaction, a kit comprises a set of DNA hairpins each comprising a label probe, capable of self-assembling into a plurality of DNA hairpins and label probes after hybridizing to the corresponding set of target probes. In some embodiments, a kit may include a general signal amplification component comprising biotin-(strept)avidin, antibodies, and/or tyramide signal amplification that interacts with label probes.

In some multiplex embodiments of kits, sets of paired target probes for two or more different RNA molecules are included. In some embodiments, kits can target 24 or more unique nucleic acid molecules using panels of up to four different sets of paired target probes in increments of up to four. In some embodiments, each set of target probes can have a unique set of hybridization-based amplification components such as a unique set of preamplifiers, amplifiers and/or label probes, and/or a set of unique DNA hairpins. In some embodiments, each set of up to four unique paired target probes may be hybridized to a tissue sample and imaged, target probes or amplification components may be removed, and the next set of up to four paired target probes can be hybridized and imaged. In some embodiments, the aforementioned steps may be repeated until all sets of target paired probes are hybridized and imaged. In some embodiments, each set of up to four unique target probes are hybridized to subsequent tissue sections that are then combined into a single image.

In some embodiments, a kit may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit aspects. In addition, a kit may include a sample that is a negative or positive control for copy number or expression of one or more biomarkers.

Any aspect of the disclosure involving specific biomarker by name is contemplated also to cover aspects involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

When applicable, we have included information about the experimental design, sample size, rules for stopping data collection, selection of endpoints, experimental replicates, and randomization for each individual experiment in the Materials and Methods below.

### Example 1—Materials and Methods

Cell Culture—Growth conditions for T47D (ATCC Cat #HTB-133, RRID:CVCL_0553) and MCF7 (ATCC Cat #HTB-22, RRID:CVCL_0031) cells are described in Lei et al. (7) and detailed in Supplementary information. ERα ligands (E2 and fulvestrant) were purchased from Sigma (E4389) and Selleckchem (S1191), respectively.

Subcloning of ESR1 mutants into a lentiviral expression vector—Lentiviral vectors expressing C-terminal HA-tagged YFP, truncated ESR1-e6, ESR1-WT, ESR1-Y537S, ESR1-D538G, ESR1-e6>YAP1, and ESR1-e6>PCDH11X were previously described (6,7). HA-tagged cDNAs encoding the new ESR1-e6 fusions in this study were constructed in a similar fashion as detailed in Supplementary information.

Generation of lentiviral stable ESR1 mutant expressing cell lines—Lentivirus were produced as described (6) by co-transfecting the above ESR1 cDNA lentiviral vectors with the packaging plasmids pMD2.G (RRID:Addgene_12259) and psPAX2 (RRID:Addgene_12260) into HEK293T (ATCC Cat #CRL-3216, RRID:CVCL_0063) cells using lipofectamine 2000 (Invitrogen, cat #11668-027). Transduced breast cancer cells were selected with 2 g/mL puromycin (Sigma, cat #P8833) for 7 days. Expression of various ESR1 proteins was validated using immunoblotting.

Immunoblotting and immunoprecipitation—Cells were harvested and whole cell lysates were prepared in RIPA lysis buffer as described (7) or in MIB lysis buffer (10) supplemented with 1× protease inhibitors and 1× phosphatase inhibitors (Roche) by sonication for 2 minutes. To make ER+ PDX tumor lysates, frozen PDX tumors were cryopulverized with a Covaris CP02 Pulverizer and then protein was extracted in MIB lysis buffer with sonication. Protein concentration determination and SDS-PAGE (20 g protein per lane) were performed as described (7). Immunoblotting of nitrocellulose membranes was performed as described (7). Primary and HRP-conjugated secondary antibodies employed are listed in the Supplementary information.

Immunoprecipitation was performed as described (7), using 2 mg of lysates from hormone-deprived T47D cells with or without E2 treatment (100 nM for 45 minutes). Lysates were incubated with 2 μg anti-HA tag antibody (Santa Cruz Biotechnology Cat #sc-7392, RRID:AB_627809) or mouse IgG (Cell Signaling Technology Cat #61656, RRID:AB_2799613) control, followed by capture of antibody-antigen complexes with protein A magnetic beads (Bio-Rad, cat #1614013) as described (7). Immunoprecipitated proteins, as well as 20 g of whole cell lysates (1% inputs), were analyzed by immunoblotting.

Cell growth, motility and invasion assays—Cell growth assays of different ESR1 fusion protein expressing breast cancer cells that were first hormone-deprived and then subsequently treated either with 100 nM fulvestrant in the presence or absence of 10 nM E2 for 7-10 days were performed in 96-well plates using an alamarBlue™ assay as described (7). Cell growth reading values were normalized to that of control YFP cells, −E2.

Cell motility was detected using a scratch wound assay of hormone-deprived stable cells in a 96-well ImageLock plate (Essen BioScience) that were pre-treated for 2 h with mitomycin C (50 ng/mL for T47D and 200 ng/mL for MCF7; Sigma, M4287) before wounding as described (7). Wound images were acquired every 6 hours for 72 hours by an IncuCyte camera (Essen Bioscience) in a cell culture incubator. Relative wound densities (RWD) were calculated as density in the wound area relative to that outside the wound area to account for confounding proliferation.

The cell invasion assay was performed and analyzed in a similar manner to the scratch wound assay except that cells were plated on Matrigel-coated plate. After the scratch was generated on cell monolayer, 50 μL Matrigel solution was added to the wells thus filling the scratch region and 100 μl of additional culture media containing mitomycin C.

RNA-Seq and analysis—Different ESR1 cDNA stably expressing T47D cell lines were cultured in CSS media for 5 days followed by treatment with or without 10 nM E2 for 2 days. RNA was isolated using RNeasy Mini Kit (QIAGEN, cat #74106) and treated with DNase (QIAGEN, cat #79254) to remove genomic DNA. The Genomic and RNA Profiling (GARP) Core at BCM confirmed concentration (using a NanoDrop spectrophotometer) and integrity (using an Agilent Bioanalyzer). The GARP core then made mRNA libraries and performed sequencing on an Illumina NovaSeq 6000 sequencing instrument as described in detail in Supplementary information. For RNA-Seq on isolated ER+ PDX tumors, frozen PDX tumors were cryopulverized as above and total RNA was isolated using the RNeasy kit. RNA-Seq was performed at the Human Genome Sequencing Center at BCM as described in detail in Supplementary information.

For RNA-seq analysis, paired-end 150 bp reads were aligned to the hg19 (GRCh37) reference genome using RSEM v1.2.31 (11) (RSEM, RRID:SCR_013027) and Bowtie 2 (12). Transcripts per million values calculated by RSEM were log 2 transformed and subjected to heatmap generation using Morpheus (https://software.broadinstitute.org/morpheus) (Morpheus, RRID:SCR_014975). Unsupervised hierarchical clustering and identification of differentially expressed genes in active ESR1 fusion protein expressing cells to cells expressing inactive fusions and controls are described in Supplementary information.

Whole exome sequencing (WES) and analysis—DNA was isolated from the ER+ PDX tumors using a QIAamp DNA Mini Kit (QIAGEN, cat #51304). WES data was generated by the Human Genome Sequencing Center at BCM using the Illumina platform as described in detail in Supplementary information. Tools used for somatic ESR1 ligand-binding domain (LBD) gene variant calling were Strelka2, Mutect2, and CARNAC (v 0.2.5b9) as described in Supplementary information. ESR1-e6>YAP1 fusion was detected in WHIM18 previously (6).

Reverse transcription-quantitative PCR (RT-qPCR)—RNA was isolated from hormone-deprived stable T47D cells as above with concentration determined using a NanoDrop spectrophotometer. One step RT-qPCR was conducted using 50 ng RNA incubated with SsoAdvanced Universal SYBR Green Supermix (Bio-Rad, cat #1725274), iScript reverse transcriptase (Bio-Rad, cat #170-8891) and 0. μM primers (Sigma) as described (7). All samples were run in triplicate on a CFX96 thermal cycler (Bio-Rad).

Immunofluorescence—Immunofluorescence was performed of different HA-tagged ESR1 fusion proteins expressed in hormone-deprived T47D cells as described (7). These proteins were detected using an anti-HA antibody (Cell Signaling Technology Cat #2367, RRID:AB_10691311, 1:50) and goat anti-mouse IgG secondary antibody (Alexa Fluor 568, Molecular Probes Cat #A-11004, RRID:AB_2534072, 1:1000) as described in Supplementary information. Nuclei were detected by DAPI staining as described (7).

PDX models—The PDX models were previously described (6,13,14). All animal procedures were approved by the Institutional Animal Care and Use Committee at BCM (protocol #AN-6934). Two-three mm pieces from PDX tumors were engrafted into cleared mammary fat pads of 3 to 4-week-old ovariectomized SCID/beige mice (Charles River). Mice were randomized to receive sterile drinking water with or without 8 g/ml E2 supplementation (n=7-16 per PDX line per arm). Tumor volumes were measured by caliper every 3-4 days, and were calculated by V=4/3×π×(width/2)2×(length/2). Mice were sacrificed when tumors reached 1.5 cm³ or at the study end point. Tumors were harvested and frozen in liquid nitrogen for storage. Additional information on BCM and HCl PDX models is available at pdxportal.research.bcm.edu/.

Gene signature and ROC curve analysis—The signature performance was calculated as follows: Accuracy=(TP+TN)/(TP+TN+FP+FN), Sensitivity=TP/(TP+FN), Specificity=TN/(TN+FP), in which TP, true positive; TN, true negative; FP, false positive; FN, false negative. ROC curve analysis was performed using “pROC” package in R (15).

ERE DNA pulldown assays—These assays were modified from the established protocol of HeLa cell nuclear extract (NE) supplemented with recombinant estrogen receptors (16,17). Briefly, nuclear extracts were made from T47D cell lines expressing YFP or different ESR1 fusion proteins (15-25 15 cm dishes employed) exactly as published (18). Pulldown assays employed 1 mg of T47D cell NE to resuspend 60 μl Dynabeads M-280 Streptavidin that was pre-bound to 3 g biotinylated 4×ERE-E4 921 bp DNA. Incubation occurred at 4° C. with gentle rotation for roughly 2 hours, followed by pelleting beads with a magnetic rack and quick washes as described (16,17). Final beads were resuspended in 30 μl 2×SDS-sample buffer, boiled, and 30% of the final supernatants were loaded onto 4-15% gradient SDS-PAGE gels. After transfer to nitrocellulose, immunoblots were probed with N-terminal ERα (Millipore Cat #04-820, RRID:AB_1587018) or DNA-PKcs (Santa Cruz Biotechnology Cat #sc-5282, RRID:AB_2172848) antibodies with ECL-based signal detection on a Bio-Rad Imaging System.

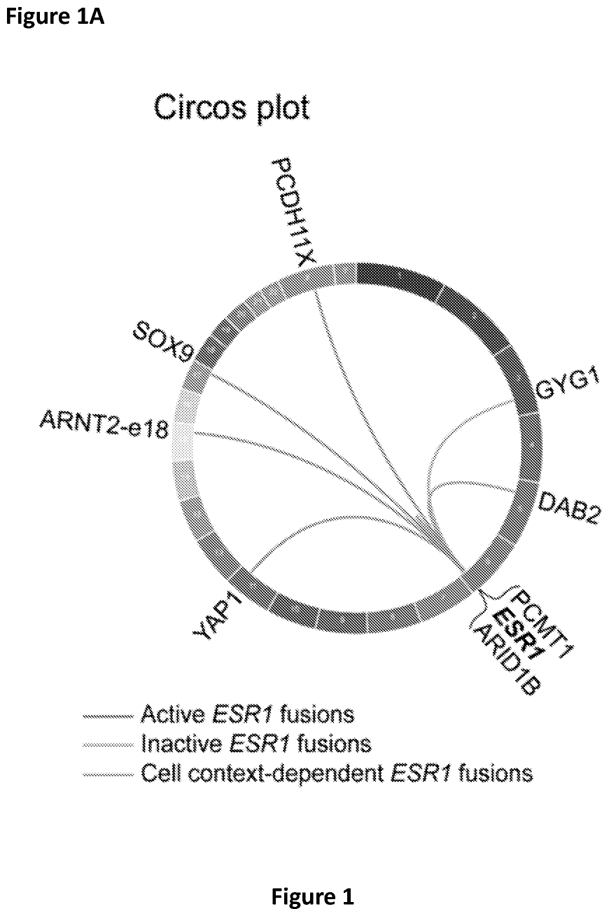

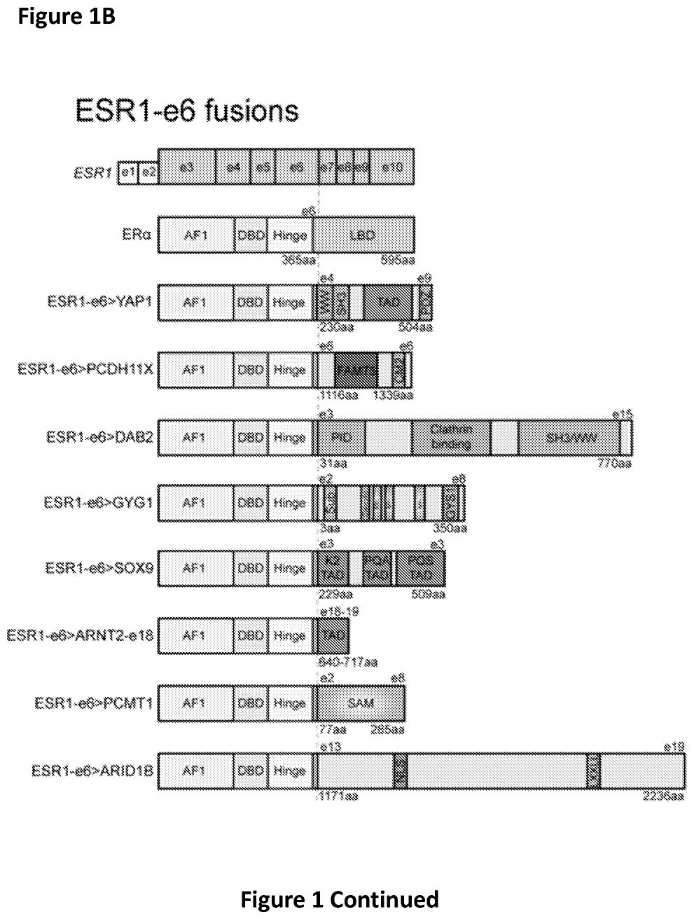

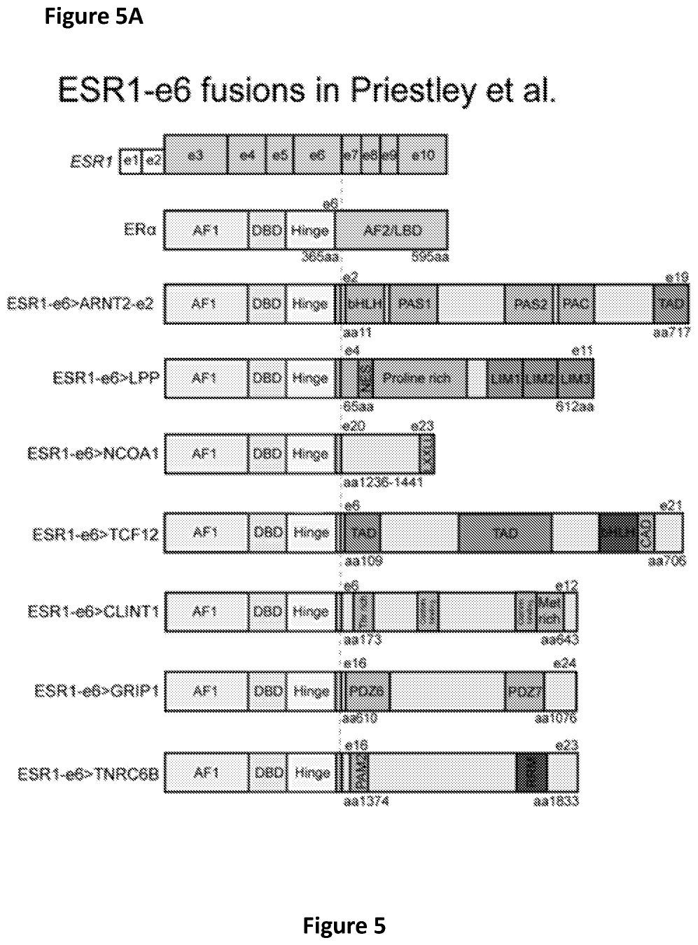

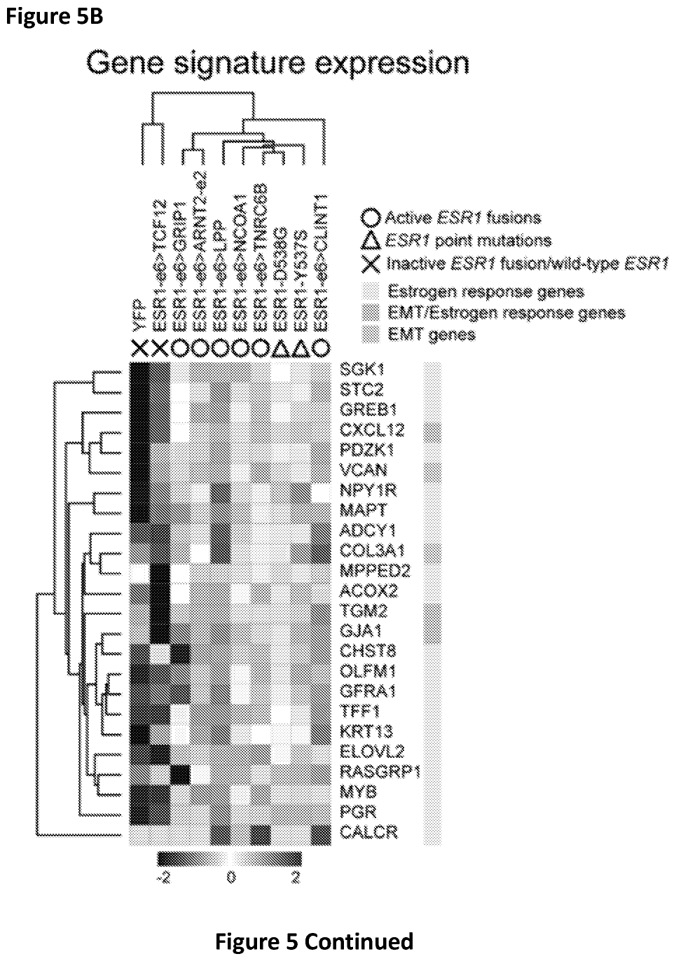

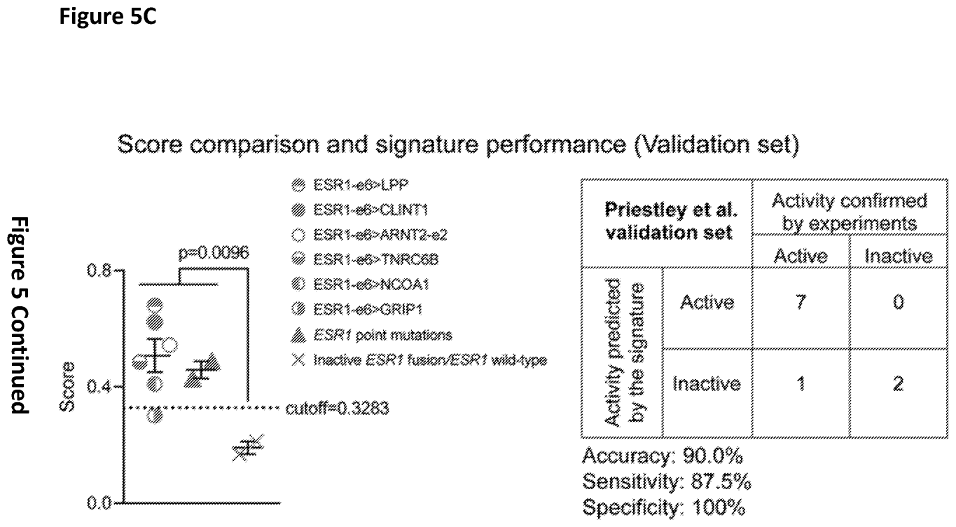

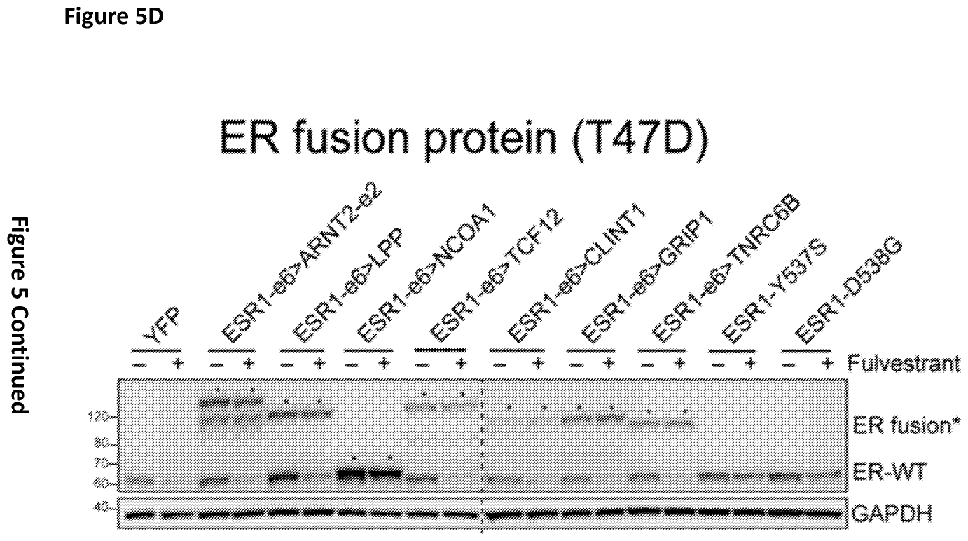

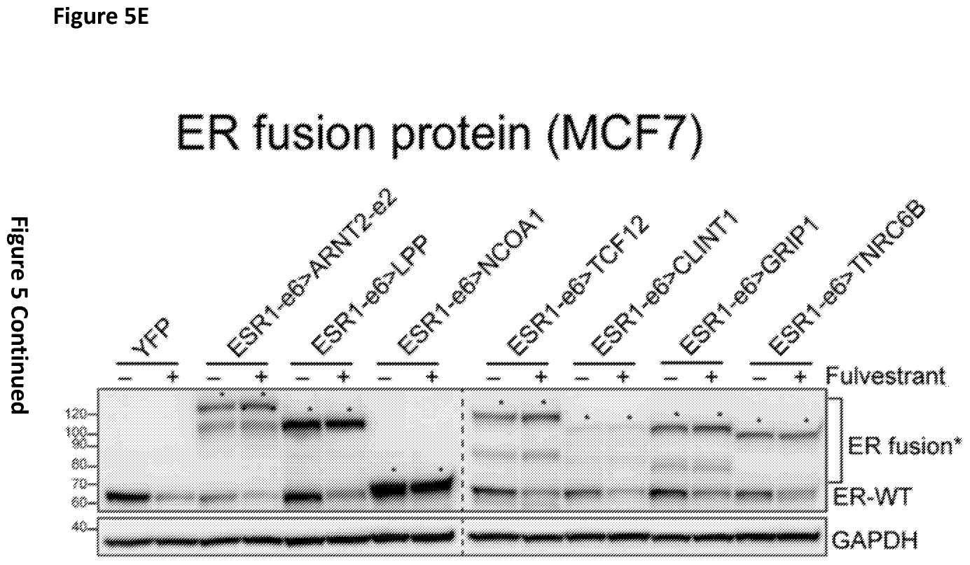

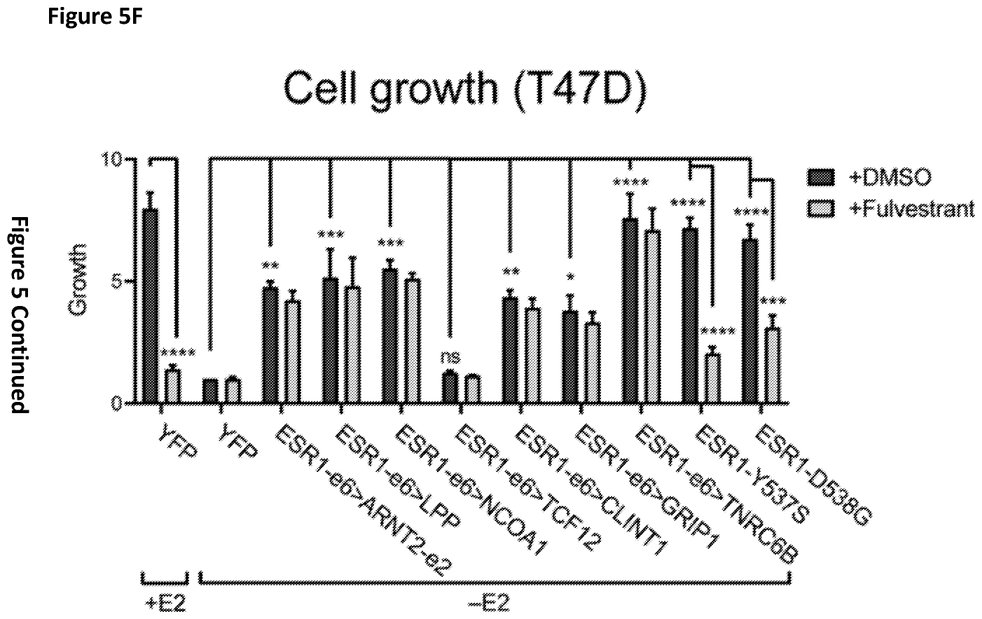

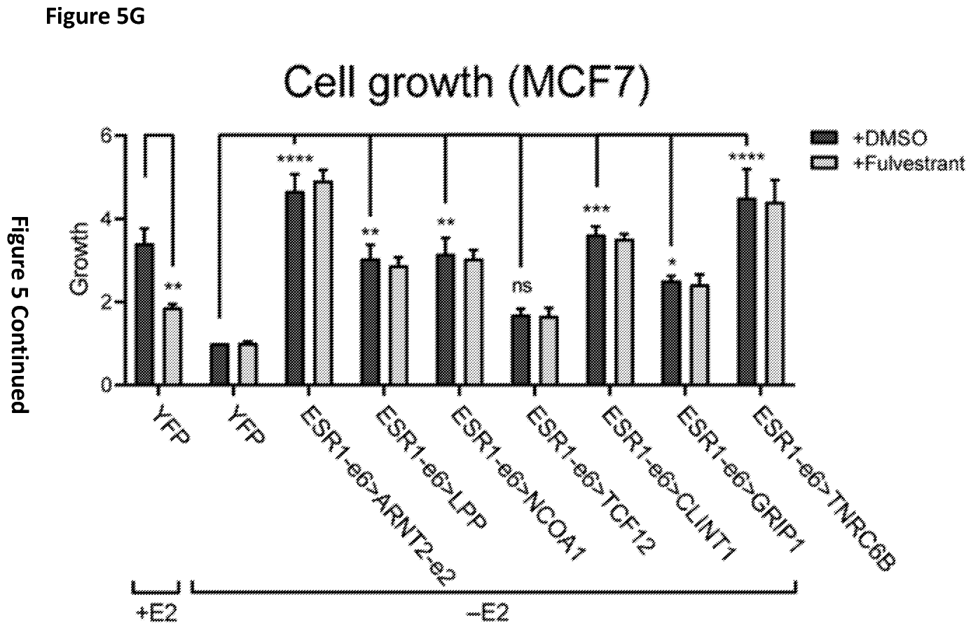

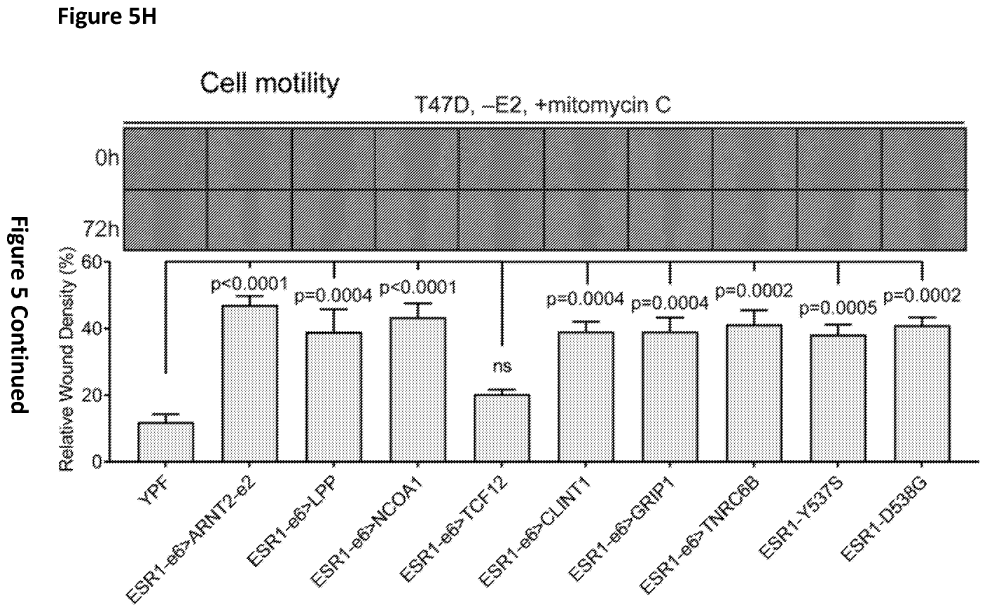

Statistical tests and analyses of publicly-available data—Unpaired two-tailed Student's t-test and ANOVA were performed with GraphPad Prism 9 (GraphPad Prism, RRID:SCR_002798), as indicated in the figure legends. P-values less than 0.05 were considered statistically significant. The protein domains with functional information in FIGS. 1B and 5A were extracted from UniProt Knowledgebase (19). RNA-Seq data derived from ER+ MBC along with ESR1 mutation status (n=55) were downloaded from the MET500 web portal (https://met500.path.med.umich.edu/). Cases with an ESR1 mRNA expression >1 FPKM were considered as ER+, using a previously described criteria (20).

### Example 2—a Subset of In-Frame ESR1-e6 Fusions Identified in ER+ MBC Patients Drive ET-Resistant Growth and Promote Hormone-Independent Motility and Invasion of ER+ Breast Cancer Cells

Six newly identified in-frame ESR1 fusions detected in samples from MBC patients were compared to the ESR1-e6>YAP1 and ESR1-e6>PCDH11× examples we described previously (7). Some fusion examples arose from inter-chromosomal translocations, such as ESR1-e6>DAB2, ESR1-e6>GYG1, ESR1-e6>SOX9 (Lee laboratory (8)) and ESR1-e6>ARNT2-e18 (Robinson, D. personal communication). Two other fusions were formed by rearrangements within chromosome 6, ESR1-e6>PCMT1 and ESR1-e6>ARID1B (Robinson, D. personal communication) (FIG. 1A). All six examples followed a structure established by the original ESR1-e6>YAP1 fusion whereby the first six exons of ESR1 were fused in-frame to C-terminal partner genes, completely replacing the ERα LBD with an alternative C-terminus. We noted two classes functionally, 1) transcription factor (TF) and transcription coactivator (CoA) fusions or 2) fusions with genes without previously established (direct) functions in gene transcription (FIG. 1B).

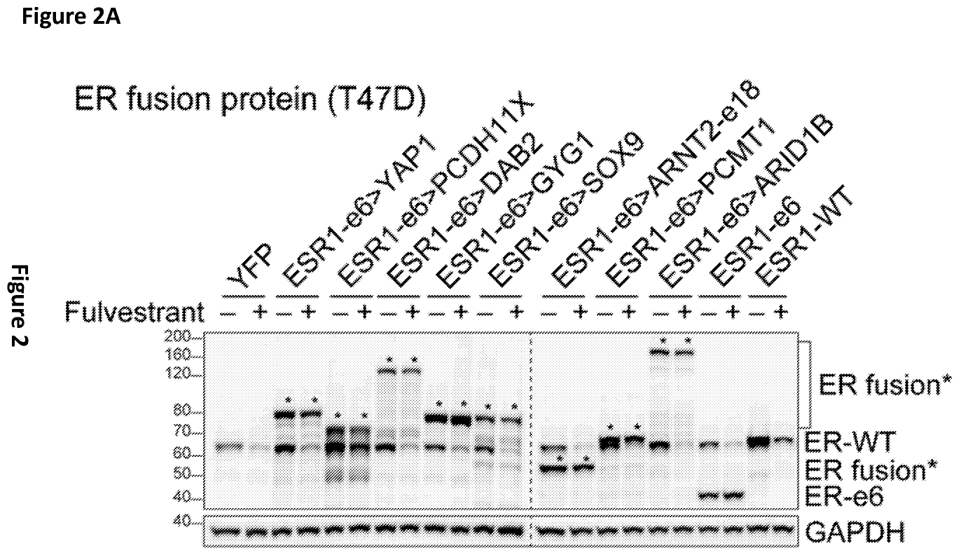

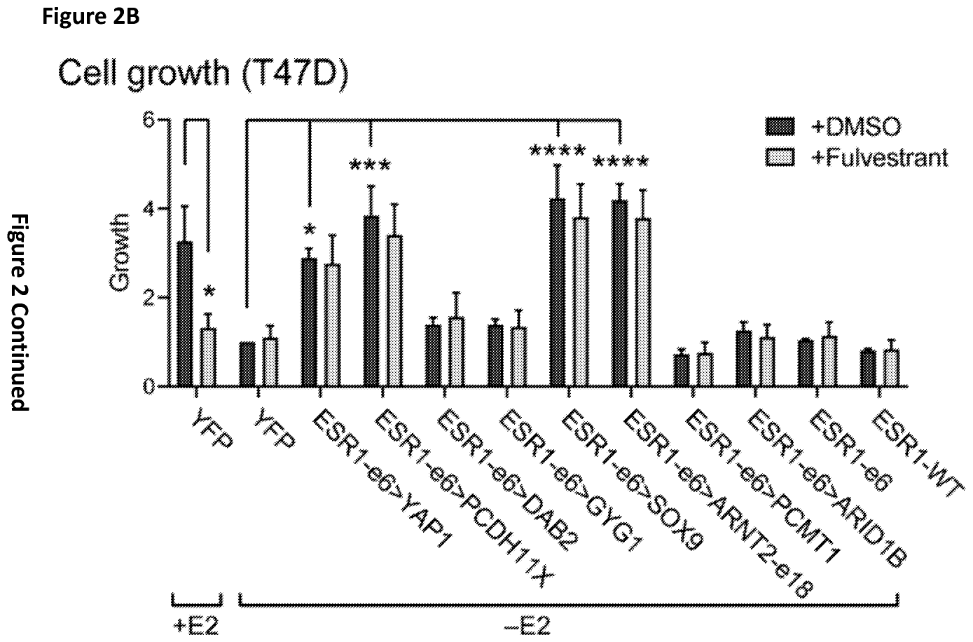

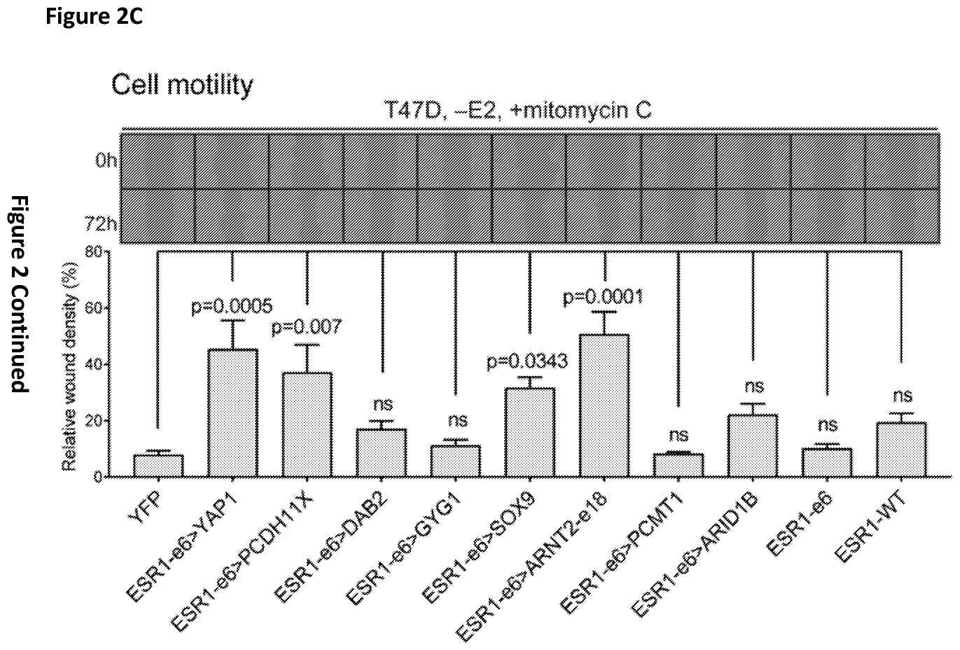

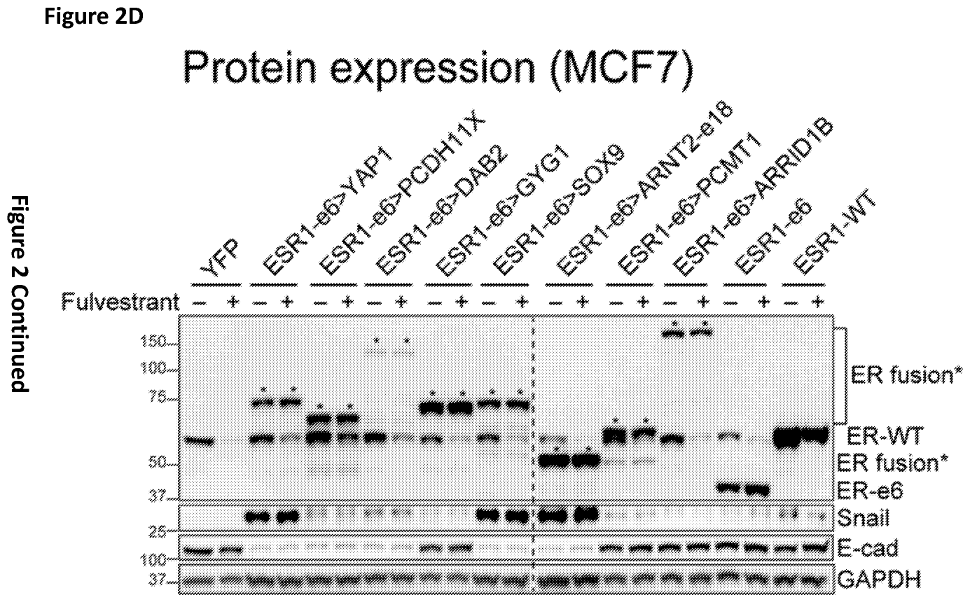

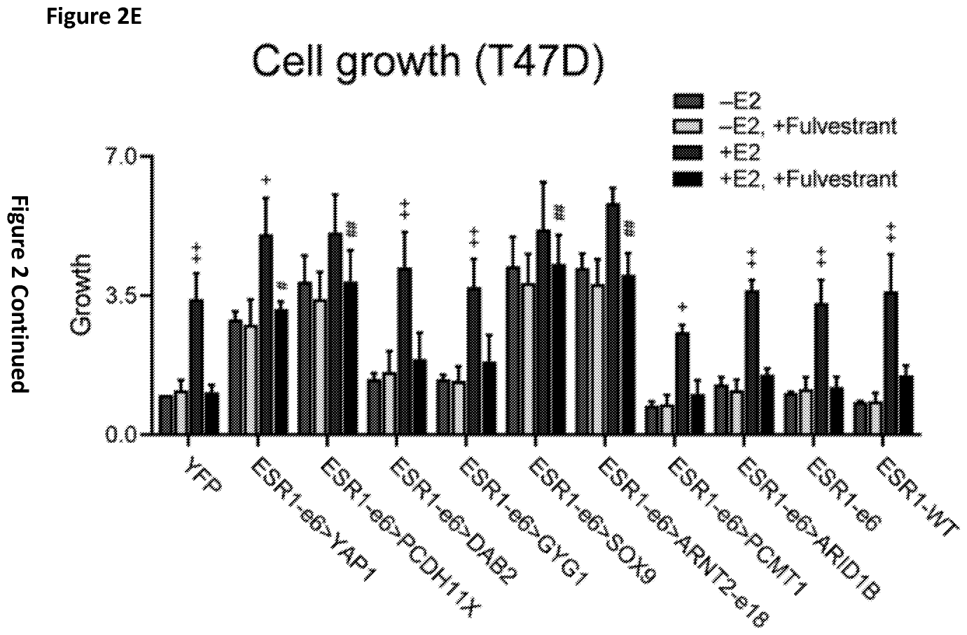

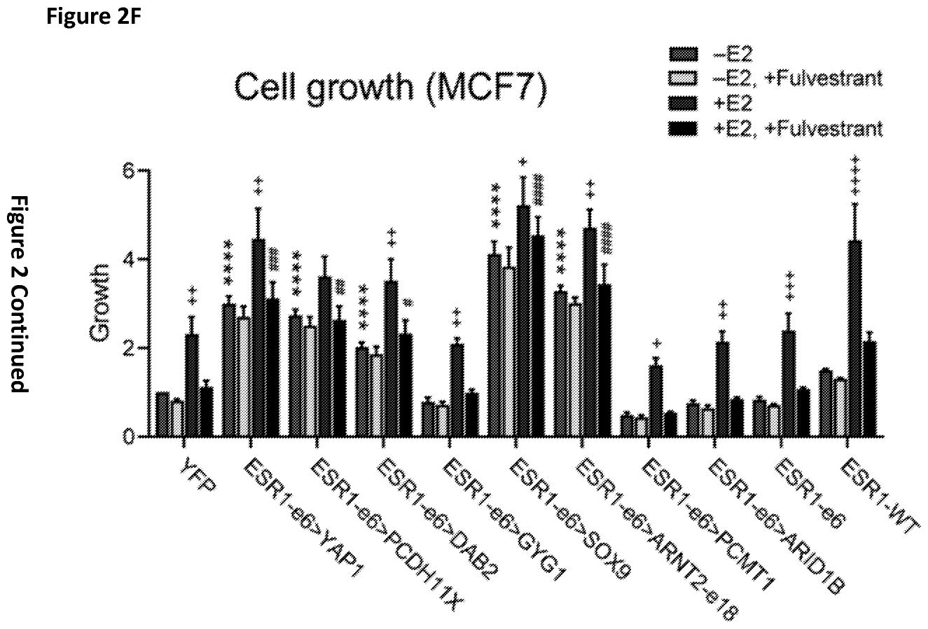

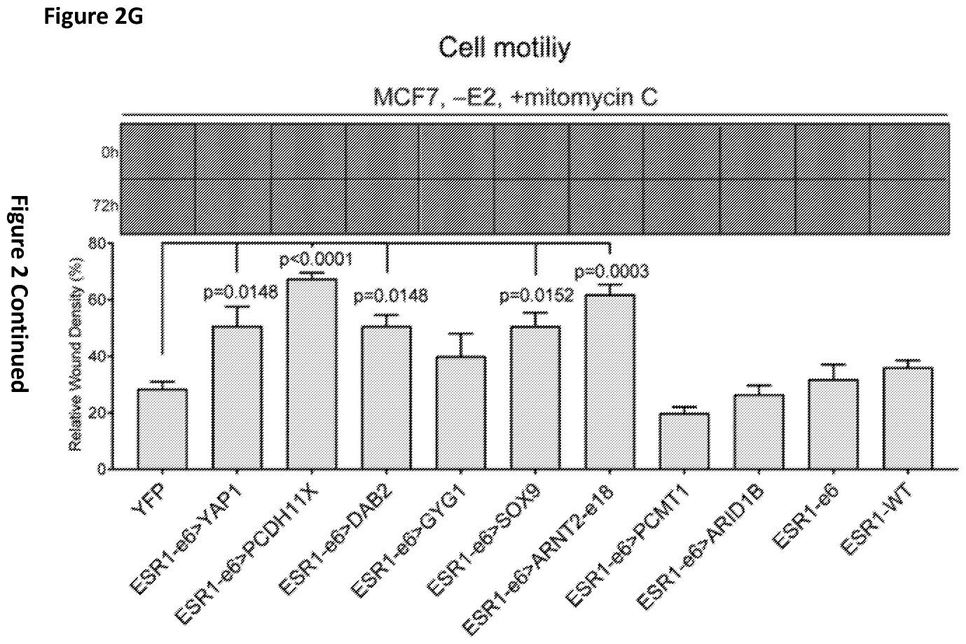

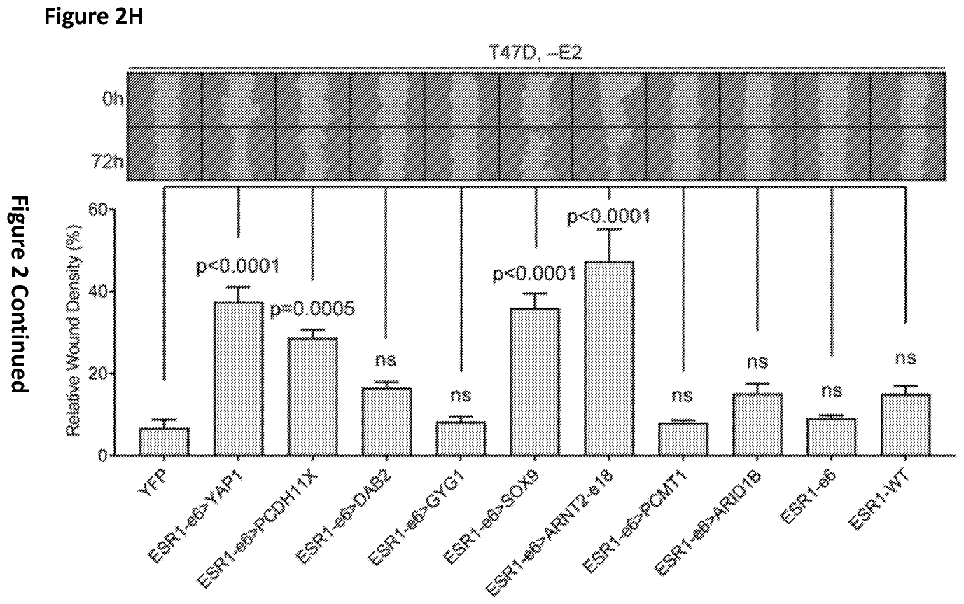

To characterize each chimeric ESR1 fusion protein, HA-tagged cDNA constructs were expressed in two ER+ breast cancer cell lines (T47D and MCF7) by lentiviral transduction. Stable cell lines expressing yellow fluorescent protein (YFP) were generated as negative controls. Truncated ESR1 (ESR1-e6 protein) and wild-type ESR1 (ESR1-WT protein) were also stably expressed to provide over-expression controls (FIGS. 2A and 2D). When cells were treated with 100 nM fulvestrant, a selective ERα degrader that inhibits endogenous ERα (21), the level of ESR1 fusion protein was predictably unaffected. In comparison, the WT ERα protein was reliably degraded, providing an endogenous control for fulvestrant activity (FIGS. 2A and 2D). To investigate whether ESR1 fusion proteins drove ET resistance, cell lines expressing ESR1 fusion cDNAs were hormone-deprived for 7 days in charcoal stripped serum-containing phenol red-free, RPMI media (CSS media) and then treated for 7-10 days with or without 10 nM E2 and with or without 100 nM fulvestrant. Cell growth was measured using an alamarBlue assay. Similar to ESR1-e6>YAP1 and ESR1-e6>PCDH11X (7), ESR1-e6>SOX9 and ESR1-e6>ARNT2-e18 conferred E2-independent growth of T47D cells compared to the YFP controls (−E2, +DMSO) (FIG. 2B, all four conditions are shown in FIG. 2E) in a manner that was uniformly resistant to fulvestrant (FIG. 2B). Although the four other ESR1-e6 fusions studied (ESR1-e6>DAB2, ESR1-e6>GYG1, ESR1-e6>PCMT1, and ESR1-e6>ARID1B) produced stable proteins, they did not promote ET-resistant growth of T47D cells with inactivity resembling the controls (truncated ESR1-e6 protein alone, ESR1-WT and YFP). The GYG1 example is an important exception, since this is an in-frame, inter-chromosomal translocation that might have been expected to be active. While cell growth was induced by E2 treatment regardless of the presence of an ESR1 fusion protein, only ESR1-e6>YAP1, ESR1-e6>PCDH11X, ESR1-e6>SOX9 and ESR1-e6>ARNT2-e18 drove significantly higher growth than YFP control cells in presence of fulvestrant (+E2, +Fulvestrant) (FIG. 2E). The elevated E2-independent, fulvestrant-resistant growth phenotypes were further validated in MCF7 cells (FIG. 2F). Interestingly, ESR1-e6>DAB2 demonstrated E2-independent, fulvestrant-resistant growth in MCF7 cells, but not in T47D cells, suggesting the activity of this fusion was cell line selective.

To determine whether each fusion protein promoted cell motility, as an initial measure of metastasis-driving potential, stable T47D or MCF7 cells were hormone-deprived and pre-treated with mitomycin-C to inhibit cellular proliferation. Cell monolayers were scratched and wound images were monitored for 72 hours. Relative wound densities (RWD) were measured as density in the wound area relative to that outside the wound area. All four growth-promoting ESR1 fusion proteins, ESR1-e6>YAP1, ESR1-e6>PCDH11X, ESR1-e6>SOX9 and ESR1-e6>ARNT2-e18, induced higher cell migration than controls in a hormone-independent manner (−E2) (FIGS. 2C and 2G). Consistent with the proliferation data, ESR1-e6>DAB2 also promoted cell motility in MCF7, but not in T47D cells (FIG. 2H). Importantly, the expression levels of the functionally active ESR1 fusion proteins were similar to the inactive examples (FIGS. 2A and 2E), suggesting that the inactivity of individual ESR1 fusion proteins was not due to differential expression or stability. ESR1-e6>YAP1, ESR1-e6>PCDH11X, ESR1-e6>SOX9 and ESR1-e6>ARNT2-e18 also promoted more invasion through Matrigel than either the controls (YFP, ESR1-e6, and ESR1-WT) or the inactive fusions (FIG. 2A-H).

### Example 3—Active ESR1 Fusion Proteins Upregulate Expression of Estrogen Response Genes and EMT Genes

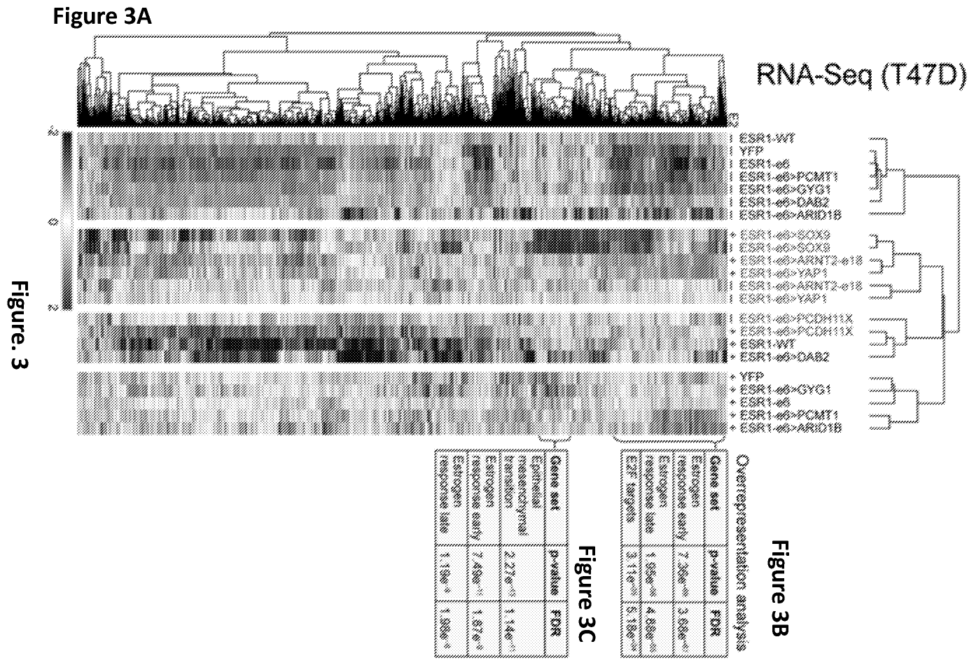

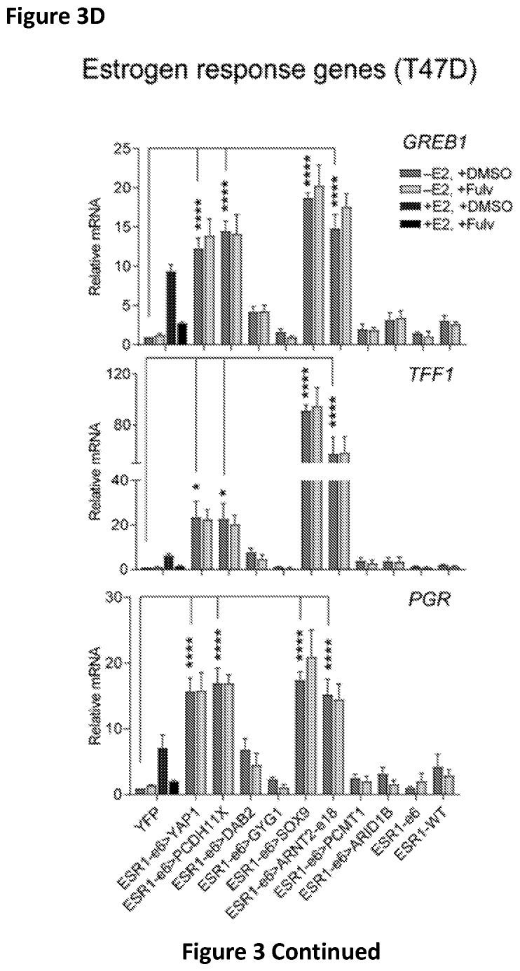

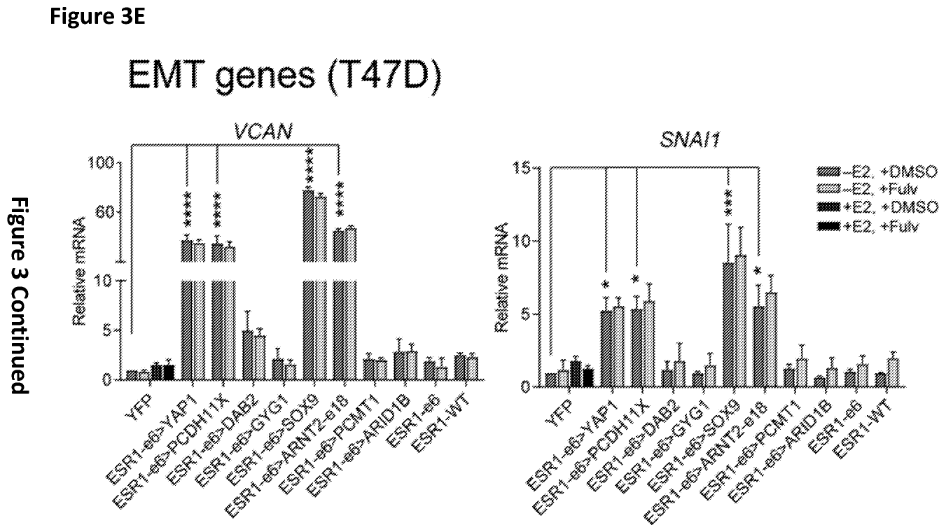

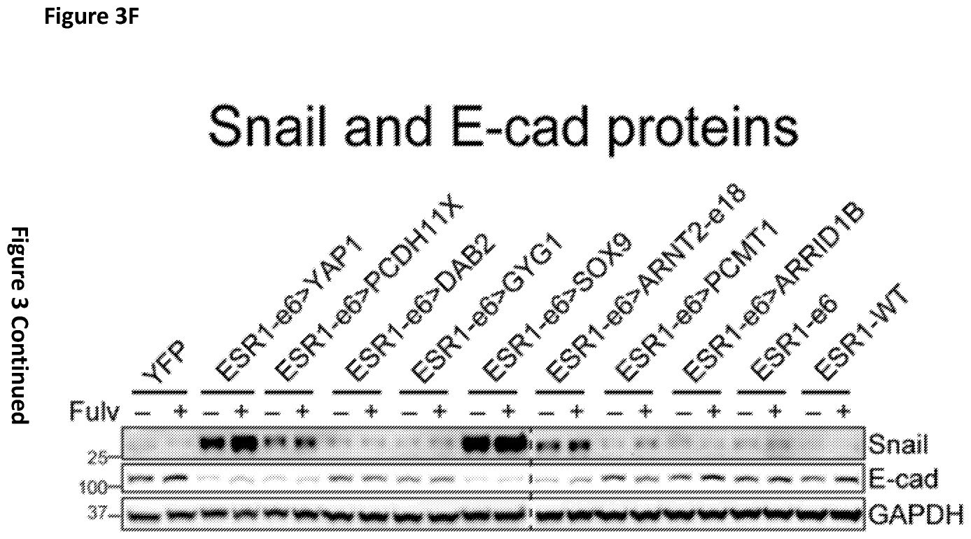

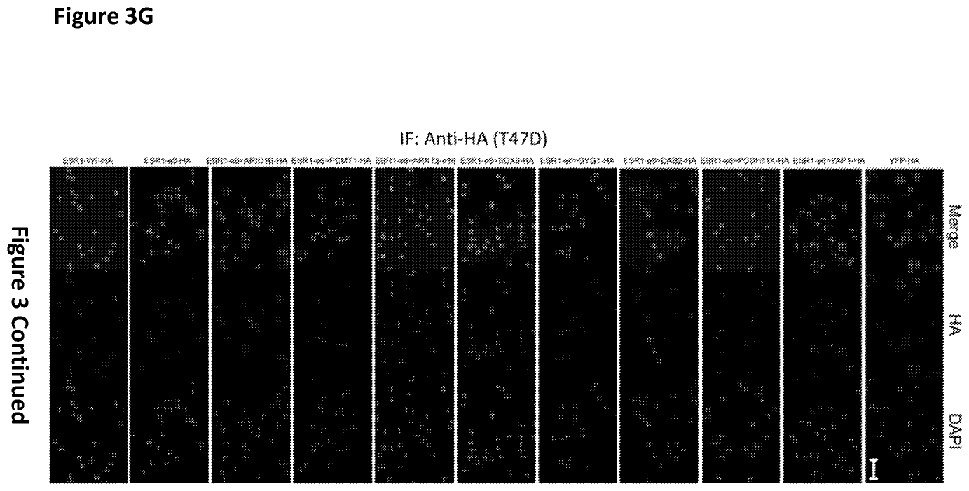

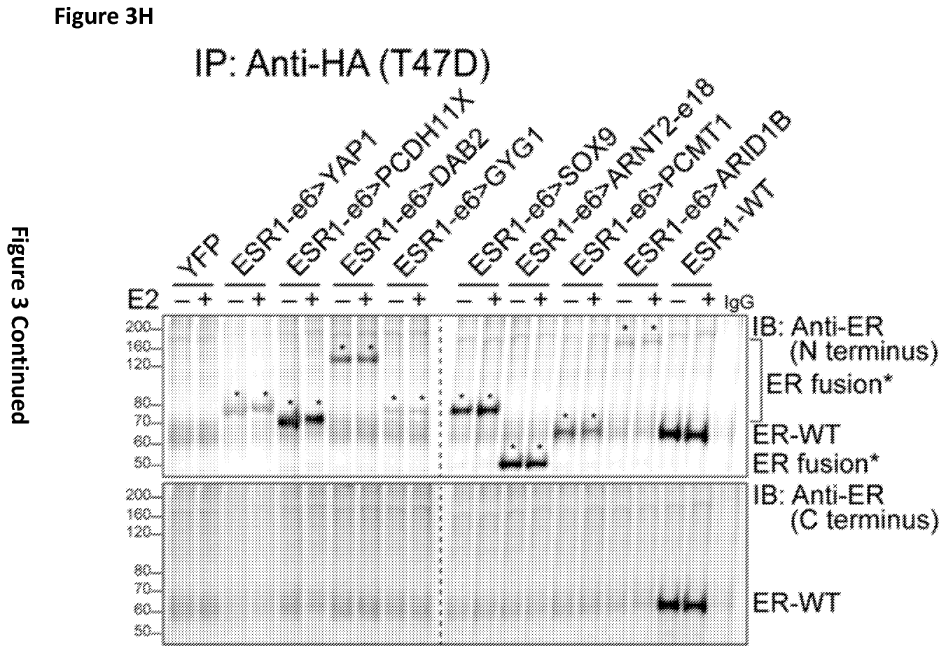

To define the transcriptional profile driven by active ESR1 fusion proteins, RNA-Seq was performed on T47D cells expressing these ESR1 fusion cDNAs as well as control (YFP, ESR1-e6, and ESR1-WT) cells in the presence and absence of E2. Hierarchical clustering showed that T47D cells expressing ESR1-e6>YAP1, ESR1-e6>PCDH11X, ESR1-e6>SOX9 and ESR1-e6>ARNT2-e18 fusions clustered distinctly from other ESR1-e6 fusions and control cells under E2-deprived conditions (−E2) (FIG. 3A). All four active fusions demonstrated an expression pattern similar to control cells treated with E2, consistent with potent hormone-independent transcriptional activation of “estrogen response” genes (FIG. 3B). Interestingly, active ESR1 fusion proteins also upregulated a cluster of genes that were not observed in the control cells stimulated by E2 (FIG. 3C). Over-representation analysis revealed a significant enrichment of “estrogen response” pathways and an epithelial-to-mesenchymal transition (EMT) signature specific to active ESR1 fusion proteins and thus consistent with data presented above on invasion and motility (FIGS. 3B and C). The expression of three canonical estrogen response genes in stably transfected T47D cells were validated using reverse transcription-quantitative PCR (RT-qPCR). ESR1-e6>YAP1, ESR1-e6>PCDH11X, ESR1-e6>SOX9 and ESR1-e6>ARNT2-e18 significantly induced the expression of GREB1, TFF1, and PGR mRNA, all three known as direct ERα targets (22) (FIG. 3D), in a hormone-independent, fulvestrant-resistant manner compared to YFP controls. These transcriptionally active ESR1 fusion proteins also upregulated two EMT-related genes, SNAI1 (Snail), encoding a master TF that induces EMT (23) by transcriptional repression of epithelial genes such as E-cadherin (24), and VCAN (versican) (FIG. 3E). The elevated expression of Snail protein and a corresponding decrease of E-cadherin (E-cad) were confirmed by immunoblotting (FIG. 3F). As expected, the expression of these genes were unaffected by fulvestrant treatment. The other ESR1-e6 fusion examples, ESR1-e6>DAB2, ESR1-e6>GYG1, ESR1-e6>PCMT1, and ESR1-e6>ARID1B did not induce E2-independent activation of ERα target genes and EMT-related genes in T47D cells. The induction of Snail protein was also reproduced in MCF7 cells, however ESR1-e6>PCDH11X displayed a minor upregulation compared to other transcriptionally active fusions (FIG. 2D). Consistent with the observed MCF7 cell line-selective increase in cell growth and migration, ESR1-e6>DAB2 also upregulated Snail expression compared to YFP control and the inactive fusions. Consistent with above T47D cell line data, E-cadherin protein was reduced in MCF7 cells expressing active ESR1 fusion proteins (FIG. 2D).

Additional experiments were conducted to demonstrate that inactive ESR1 fusion proteins enter the nucleus as the nuclear translocation signal is preserved. Also there is no biochemical evidence for heterodimer formation with WT ERα (FIG. 3G-3I). These data imply that the inactive ESR1 fusion genes are not dominant negative, a result consistent with the normal E2-induced growth in inactive fusion expressing cells.

### Example 4—Active ESR1 Fusion Proteins Induce a Characteristic, Hormone-Independent Transcriptional Signature

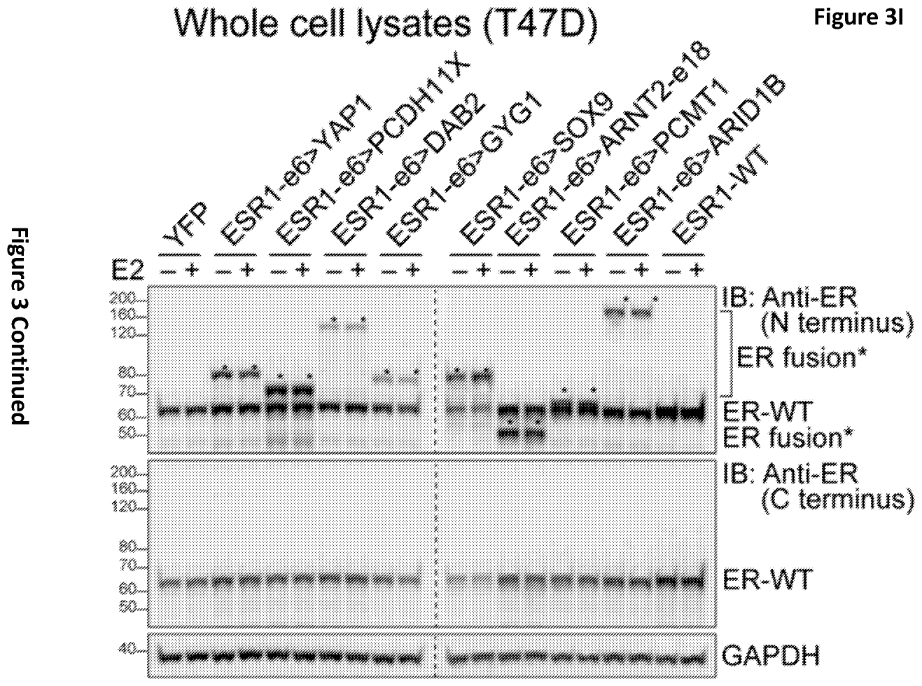

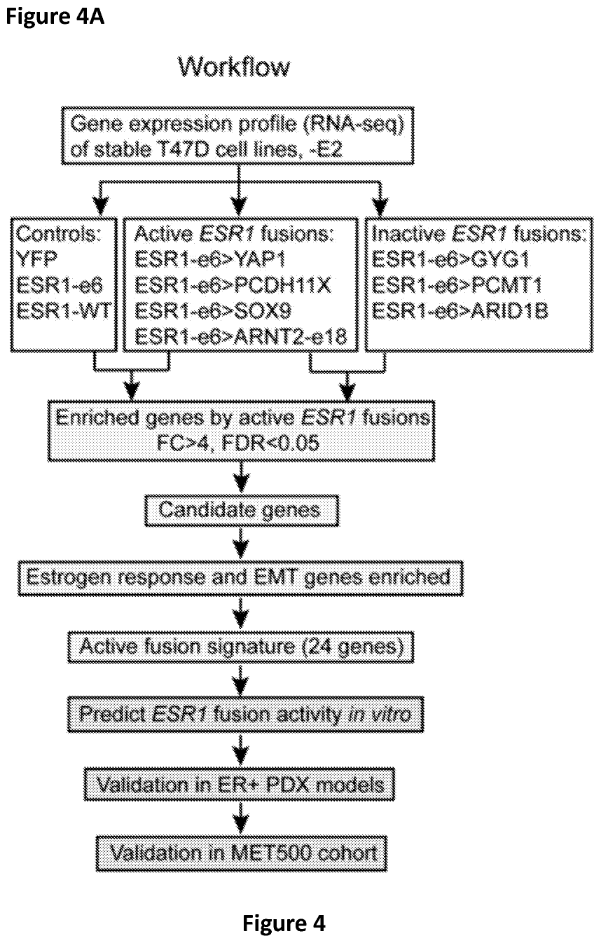

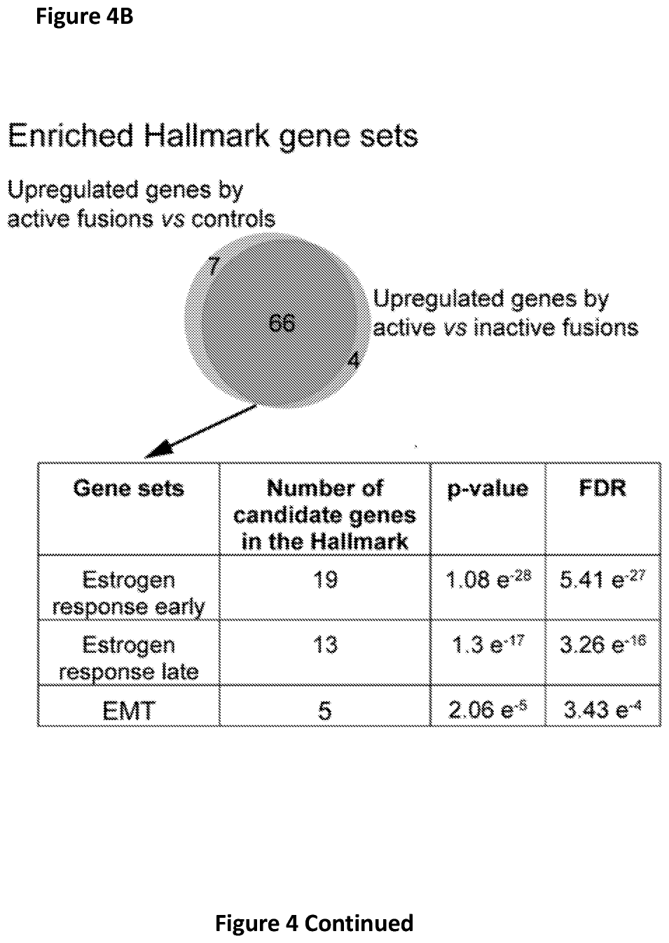

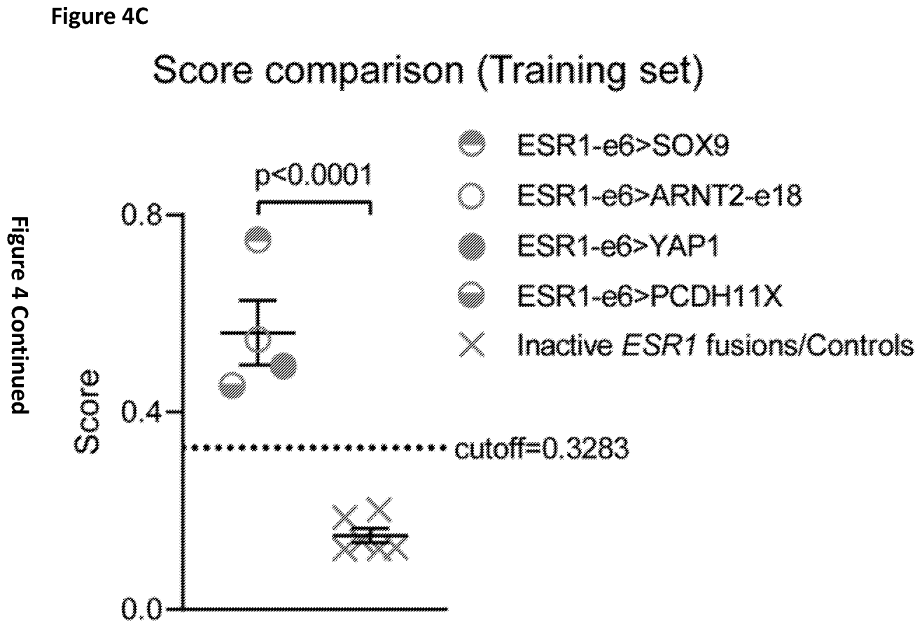

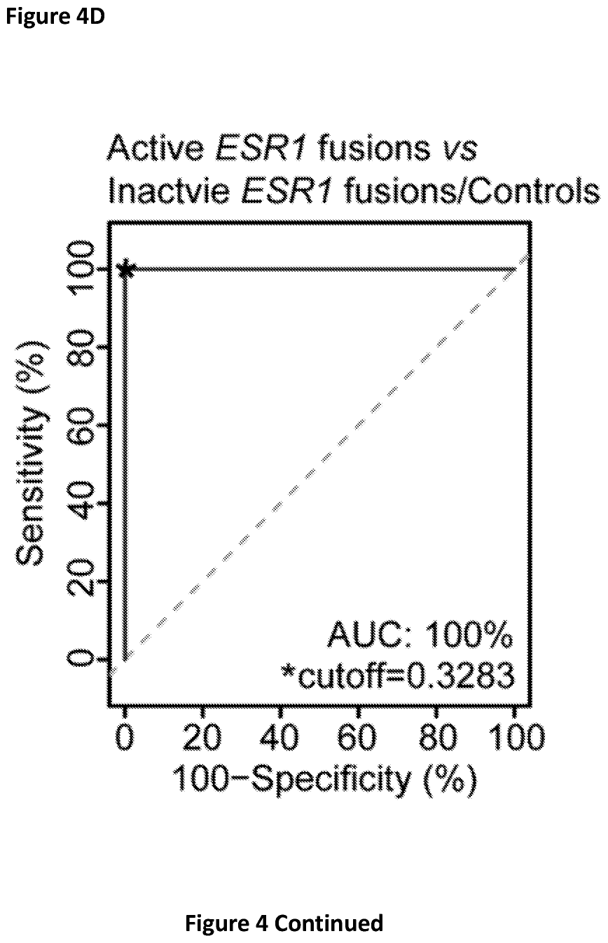

The 3′ partners of ESR1 fusion genes are highly diverse, consequently their presence is only revealed by unbiased genomic techniques such as whole genome sequencing or RNA-Seq. These techniques are not routinely used clinically, and it is currently unknown how sensitive unbiased techniques are as screens for an ESR1 gene fusion event, because an orthogonal assay is required to determine sensitivity. Adding to diagnostic complexity, some ESR1 fusion proteins are inactive and therefore not clinically actionable. An in vitro assay such as the ones described above are feasible but difficult to conduct within a clinically useful time-frame. We therefore sought to develop a gene expression signature that is diagnostic for the presence of a transcriptionally active ESR1 fusion protein. RNA-Seq was applied to T47D cells expressing ESR1 fusion cDNAs to identify genes that were selectively upregulated by the four transcriptionally active ESR1 fusion proteins as compared to: 1) three inactive ESR1 fusions and 2) three controls (FIG. 4A). These two comparisons yielded an overlapping group of 66 candidate genes with a fold change (FC) greater than 4 and a false discovery rate (FDR) less than 0.05. (see Table 1) Over-representation analysis using Hallmark pathways from MSigDB (25,26) identified candidate genes that were overrepresented in the estrogen response (early and late) and EMT gene sets (FIG. 4B). An exemplary active ESR1 fusion signature was then devised based on estrogen response and EMT genes, as these were the top two pathways modulated by expression of an active ESR1 fusion protein. Specifically, the exemplary active ESR1 fusion signature comprises 24 Hallmark genes, including 19 genes in the estrogen response set (CHST8, MAPT, OLFM1, PDZK1, RASGRP1, MPPED2, GREB1, MYB, GFRA1, PGR, ELOVL2, ADCY1, NPY1R, TFF1, ACOX2, SGK1, STC2, CALCR and KRT13), two genes in the EMT gene set (VCAN and COL3A1), and three genes in both gene sets (CXCL12, GJA1 and TGM2). The expression of each gene was ranked by percentile within each sample and scores were computed as the mean percentile of the signature gene sets. ESR1 fusions were predicted as encoding active or inactive proteins according to the cutoff obtained by the receiver operating characteristic (ROC) curve analysis (cutoff, 0.3283) (FIG. 4D). In this training set, transcriptionally active ESR1 fusion proteins showed significantly higher scores as compared to inactive fusions and controls, as expected (FIG. 4C).

TABLE 1
66 Upregulated Active ESR1 Fusion Biomarker Genes

| | | | | | |
|---|---|---|---|---|---|
| ACOX2 | CD34 | GFRA1 | MPPED2 | RASGRP1 | SPINK5 |
| ADCY1 | CHST8 | GJA1 | MYB | RBM24 | STC1 |
| ADRA2A | COL3A1 | GREB1 | NKAINI | RIMS4 | STC2 |
| AFF3 | CT62 | GREM2 | NPYIR | ROBO3 | SUSD3 |
| AMZI | CXCL12 | HEY2 | NXPH3 | SEMA3A | SYTL5 |
| BFSP2 | DOK7 | IFITM10 | OLFM1 | SERPINA6 | TFF1 |
| BMPRIB | DSCAMLI | IGF2 | PDZKI | SGK1 | TGM2 |
| C14orf182 | ELOVL2 | KCNHI | PGLYRP2 | SLC47A1 | UGT3A2 |
| CALCR | FLT4 | KRT13 | PGR | SOX5 | VCAN |
| CCDC88A | FMN1 | MAPT | PPP2R2C | SPINK13 | WT1 |
| CD109 | GATA4 | MDGA1 | PRSS56 | SPINK4 | ZNF385B |

### Example 5—an Exemplary 24-Gene Transcriptional Signature Predicts the In Vitro Activity of Additional ESR1-e6 Fusion Genes

To validate the exemplary 24-gene ESR1 fusion activity signature, we studied seven additional ESR1 gene fusions published by Priestly et al. (27). These in-frame ESR1-e6 fusions were identified in ER+ MBC patients by whole-genome sequencing, including four fusions with TF or CoA partners, ESR1-e6>ARNT2-e2, ESR1-e6>LPP, ESR1-e6>NCOA1 and ESR1-e6>TCF12 (FIG. 5A). Another three fusions, analogous to PCDH11X, involved genes encoding protein-protein interaction motifs that serve non-transcriptional cellular functions, including ESR1-e6>CLINT1, ESR1-e6>GRIP1 and ESR1-e6>TNRC6B. The same approach as in FIG. 2 was taken to assess the function of these new fusions in vitro. All of the seven ESR1 fusion cDNAs expressed stable chimeric proteins in T47D and MCF7 cells (FIGS. 5D and 5E). Three fusions that involve TF/CoA partners, ESR1-e6>ARNT2-e2, ESR1-e6>LPP, and ESR1-e6>NCOA1, drove E2-independent and fulvestrant-resistant growth, as well as increased motility of T47D cells, when compared to the YFP controls (−E2, +DMSO) (FIG. 5F-5H). Surprisingly the fourth fusion, ESR1-e6>TCF12, which involves a TF in the basic helix-loop-helix (bHLH) E-box family, expressed a stable chimeric protein, but was inactive in both T47D and MCF7 cells (FIG. 5F-5H). The ESR1-e6>TCF12 fusion was able to bind to concatenated EREs in a pulldown assay similar to active fusion examples (ESR1-e6>YAP1, ESR1-e6>SOX9 and ESR1-e6>CLINT1) (FIG. 5I), thus suggesting that the transcriptional inactivity of ESR1-e6>TCF12 was not due to lack of an ability to bind DNA.

Three gene fusions that did not involve a known TF/CoA partner, ESR1-e6>CLINT1, ESR1-e6>GRIP1 and ESR1-e6>TNRC6B, but all demonstrated ET-resistant cell growth and enhanced E2-independent motility, although the effect of ESR1-e6>GRIP1 on proliferation was statistically marginal (FIG. 5F-5H). RNA-Seq was then performed on RNA extracted from T47D cells that expressed each new ESR1 fusion protein, as well on RNA from YFP control cells. In this experiment we also included two common ESR1 LBD point mutations (Y537S and D538G) to compare the active ESR1 fusion signature with the transcriptional profile associated with known activating ESR1 point mutants. Five out of six active ESR1 fusions (ESR1-e6>ARNT2-e2, ESR1-e6>LPP, ESR1-e6>NCOA1, ESR1-e6>CLINT1 and ESR1-e6>TNRC6B) demonstrated similar elevated expression of the exemplary 24-gene signature in sum, although there was some variability at the level of individual genes (FIG. 5B). ESR1-e6>GRIP1 induced lower expression of the exemplary 24-gene signature than other active fusion examples, consistent with its weaker activity in proliferation assays compared to the five other fusions studied. Interestingly, the two ESR1 LBD point mutant proteins expressed in T47D cells induced similar levels of gene expression from the exemplary 24-gene signature as active ESR1 fusion proteins, suggesting that despite different mutational mechanisms for ESR1 protein activation, LBD point mutants and translocated ERs activate a similar pathogenic transcriptional pattern (FIG. 5B). The mean signature scores of active ESR1 fusions and LBD point mutants were significantly increased compared to those of the inactive ESR1-e6>TCF12 fusion and YFP (endogenous ERα) control (FIG. 5C). As expected, the mean score of the weakly active ESR1-e6>GRIP1 fusion fell below the cutoff value. The validation statistics of the independent Priestley et al. (27) set showed an accuracy of 90.0% (specificity, 100%; sensitivity, 87.5%) (FIG. 5C). Since the exemplary 24-gene signature was similarly induced by ESR1 LBD point mutants and active ESR1 fusion proteins, it was given the moniker “MOTERA” for Mutant or Translocated Estrogen Receptor Alpha.

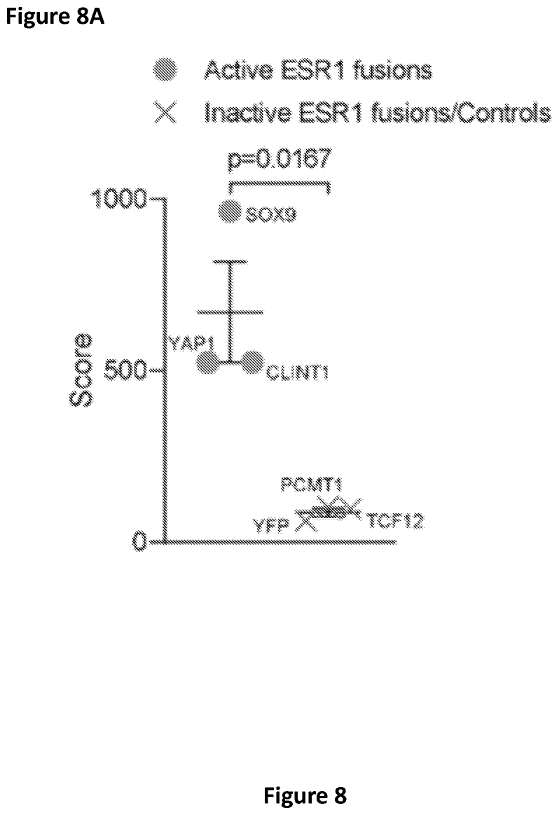

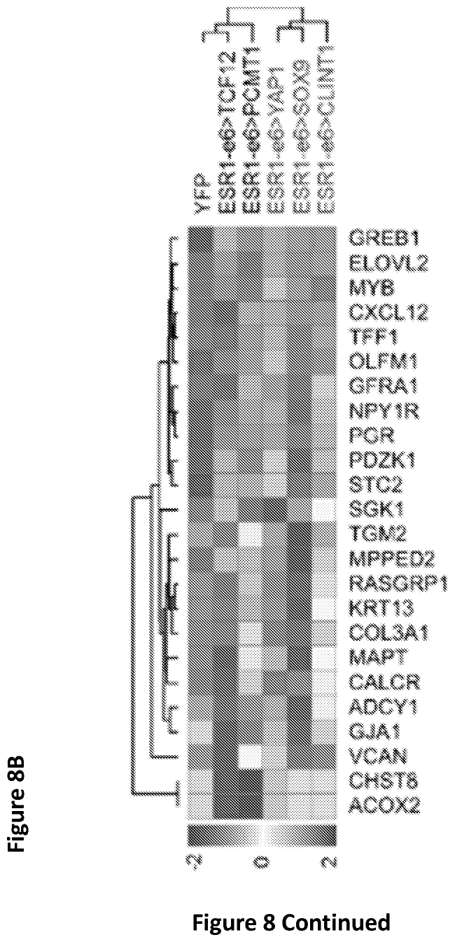

It was determined whether a 24-gene MOTERA gene expression signature induced by active, but not inactive, ESR1 fusion proteins could accurately inform ER+ MBC patients' status for subsequent treatment regimen determination. A labeled probe-based hybridization analysis assay platform (e.g., a NanoString platform) was developed that included probes for 24 MOTERA genes (ACOX2, ADCY1, CALCR, CHST8, COL3A1, CXCL12, ELOVL2, GFRA1, GJA1, GREB1, KRT13, MAPT, MPPED2, MYB, NPY1R, OLFM1, PDZK1, PGR, RASGRP1, SGK1, STC2, TFF1, TGM2, and VCAN) and 4 housekeeping genes as internal controls (B2M, GAPDH, PSMC4, and PUM1). As proof-of-principle that custom MOTERA probes can detect an elevated MOTERA signature, the inventors assayed RNA isolated from an ER+ breast cancer cell line, T47D, expressing three active ESR1 fusion proteins (YAP1, SOX9 and CLINT1) as compared to YFP control and two inactive ESR1 fusions (PCMT1 and TCF12). The labeled probe-based hybridization analysis probes (e.g., NanoString probes) for an exemplary MOTERA signature genes and control genes targeted SEQ ID NO: 1 to SEQ ID NO: 28, assays comprising these probes successfully detected significantly higher MOTERA scores in active ESR1 fusions when compared to control YFP and inactive ESR1 fusion expressing cells (FIGS. 8A and 8B).

Alternative internal control genes and/or number of internal control genes can be used, and the number of MOTERA signature genes can be reduced to a subset (e.g., 6, 10, 14, 18, or 22, or any range derivable therein) of the MOTERA genes described herein.

### Example 6—an Exemplary MOTERA Signature Accurately Predicts the Presence and Functional Status of ESR1 Mutations and Gene Fusions in ER+ PDX Tumors and Clinical Samples

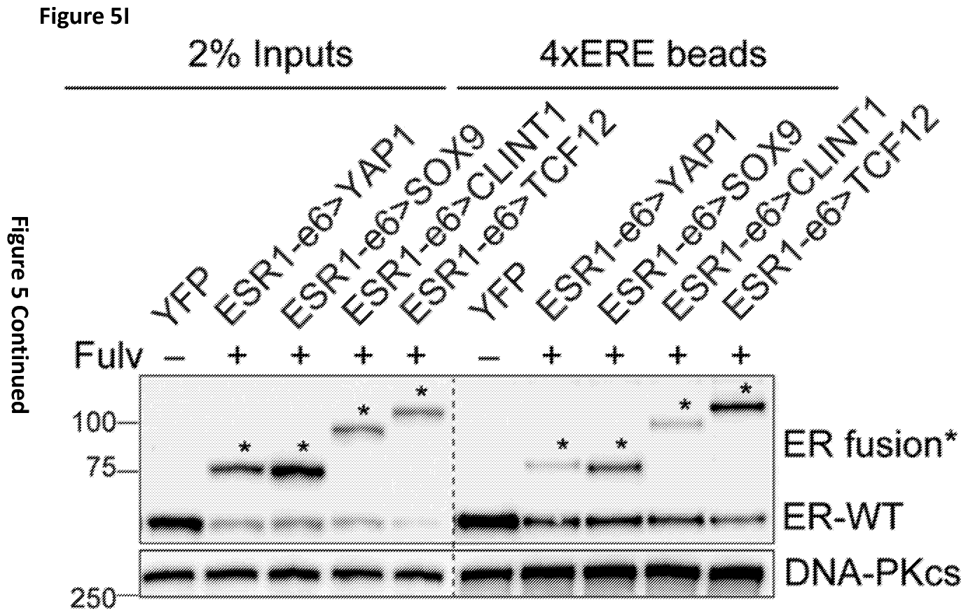

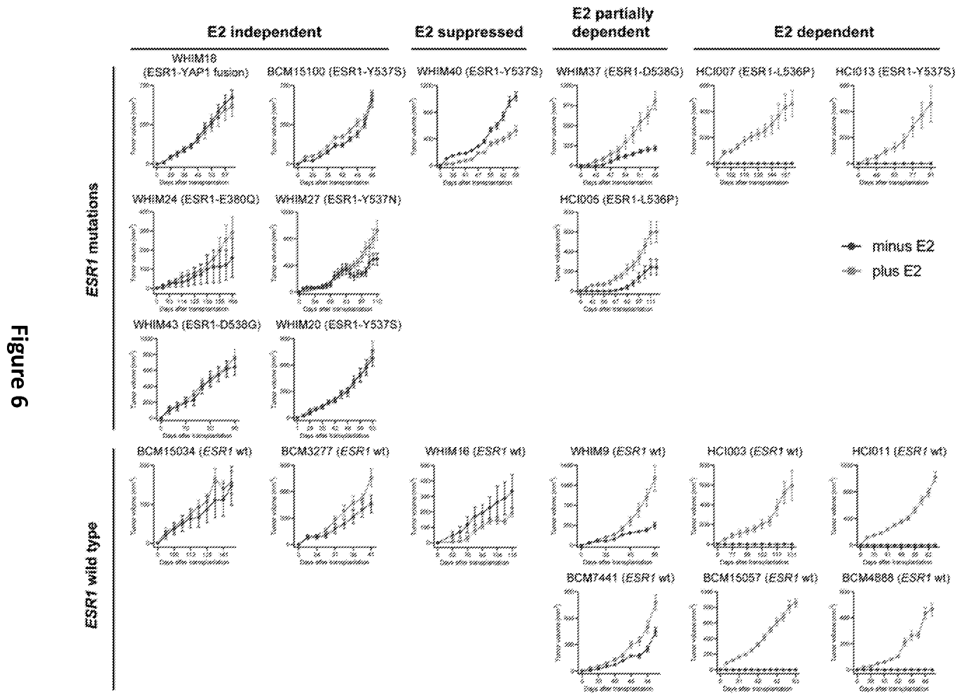

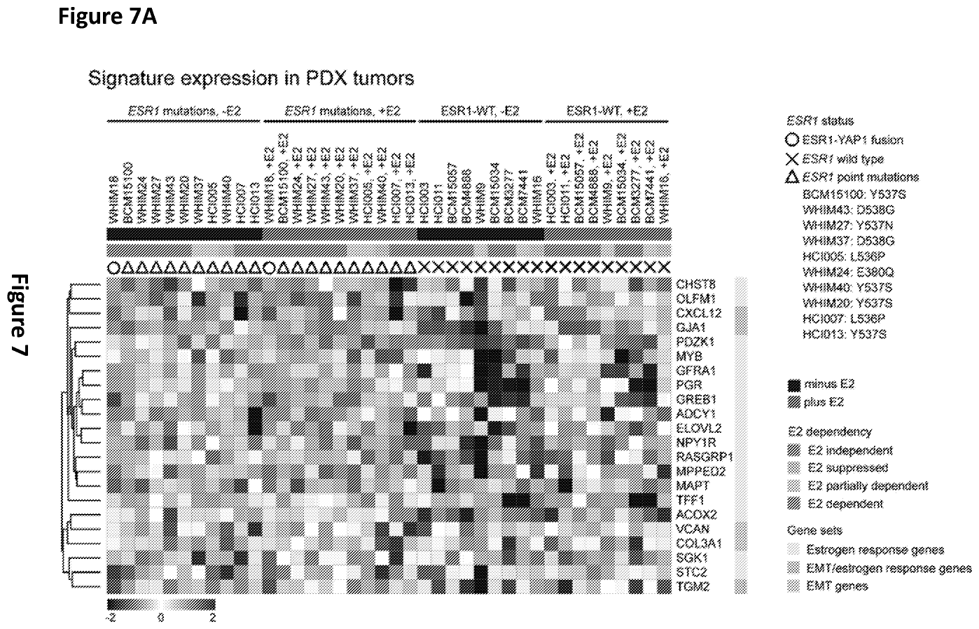

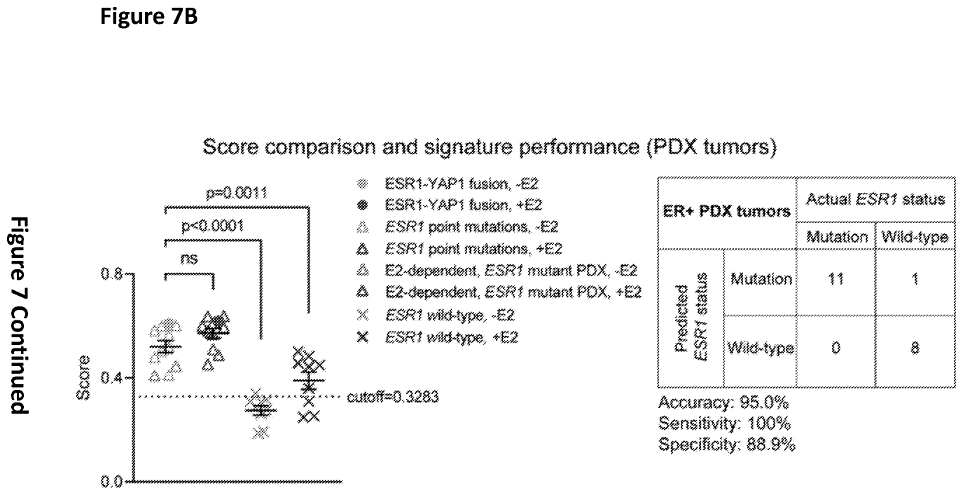

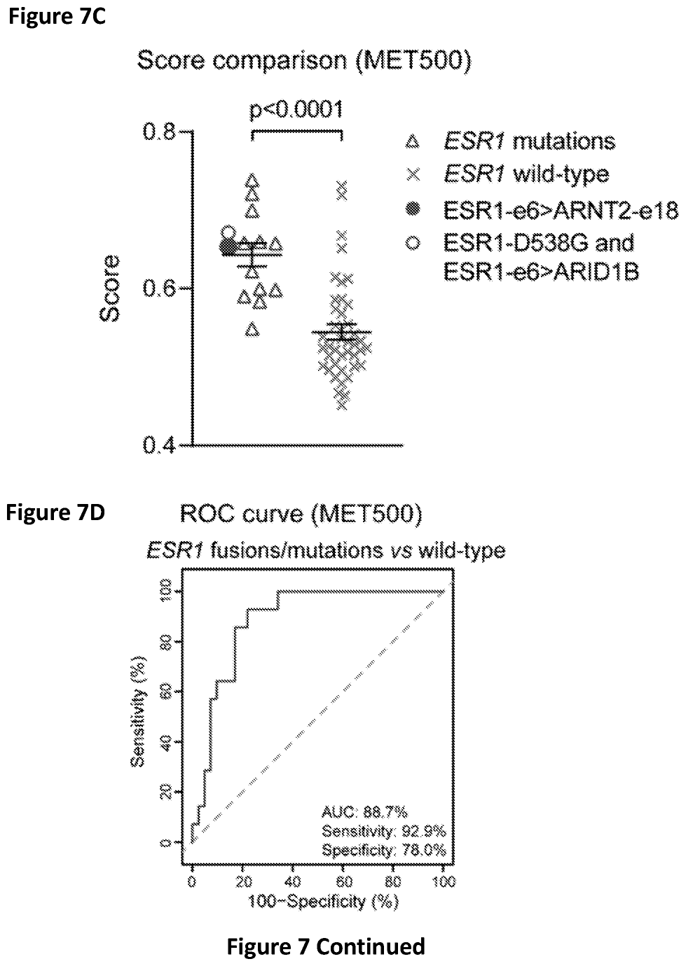

To test the properties of the exemplary MOTERA signature in human tumors that naturally express either ESR1 gene fusions or ESR1 LBD point mutations, we examined performance for each expressed ESR1 fusion or ESR1 LBD point mutant detection in a panel of 20 ER+ PDX tumors. The E2 dependence of each PDX tumor was evaluated in ovariectomized SCID/beige mice with or without 8 g/ml E2 in the drinking water (FIG. 6). ESR1 mutation status was determined by whole exome sequencing (WES) and gene expression was determined by RNA-Seq under both plus estradiol (+E2) and minus estradiol (−E2) conditions. When tumors were completely E2 dependent, the −E2 transcriptome was established by replacing the +E2 water with control (−E2) water for one week and then harvesting the tumors. As expected, the exemplary MOTERA signature was highly expressed in the E2-independent WHIM18 PDX naturally expressing the ESR1-YAP1 fusion protein (6) (FIG. 7A), thus demonstrating a high degree of similarity between the experimental context of the ESR1-e6>YAP1 cDNA in T47D cells and the natural context in a PDX where this fusion was first identified. Consistent with T47D-based gene expression findings displayed in FIG. 5B, ET-resistant PDXs bearing a variety of ESR1 LBD point mutations also induced the exemplary MOTERA signature, confirming an overlap between the transcriptional properties of active ESR1 fusion proteins and LBD point mutants noted in T47D cell experiments (FIG. 7A). For example, the exemplary MOTERA signature score was enriched over the cutoff derived from the T47D training set in the cases of BCM15100, WHIM20, WHIM40, and HCI013 (all expressing ESR1-Y537S), WHIM37 and WHIM43 (expressing ESR1-D538G), WHIM24 (expressing ESR1-E380Q), WHIM27 (expressing ESR1-Y537N), and HCI005 and HCI007 (expressing ESR1-L536P) mutants (FIG. 7B). PDX tumors expressing ESR1-WT (HCI003, HCI011, BCM15057, BCM4888, BCM15034, BCM3277, BCM7441, WHIM9 and WHIM16) had MOTERA scores below the cutoff in low estradiol (−E2) conditions in each case (FIG. 7B). We note that the mean signature scores for ESR1-WT tumors increased with E2, consistent with some genes in the signature being E2-induced (FIGS. 7A and B). Thus, as a screening tool, the exemplary MOTERA signature may potentially be more specific if the biopsy sample is taken while the patient is taking an AI or an anti-estrogen. Paradoxically, the HCI013 PDX example harbors the Y537S ESR1 mutation but remained E2-dependent as previously reported by Welm et al. (28) (FIG. 6). Similarly, HCI007 harbors an ESR1 L536P mutation, but also grew in an E2-dependent manner. These tumors have lower exemplary MOTERA scores but still above the training set defined cutoff. Presumably in these examples, ESR1-WT is functionally dominant over the LBD mutant ERα, although the mechanism remains obscure. Under −E2 conditions, the MOTERA signature successfully distinguished between ET-resistant tumors driven by mutant or translocated ESR1 proteins from ESR1-WT PDXs, with an accuracy of 95.0% (specificity, 88.9%; sensitivity, 100%) (FIG. 7B). Although the exemplary MOTERA transcriptional signature was largely composed of estrogen response genes, expression levels were not affected by E2 supplementation to the WHIM18 ESR1-YAP1 expressing PDX or other PDXs expressing ESR1 LBD point mutations, underscoring sensitivity for the activated ESR1 mutant/translocated protein state (FIGS. 7A and B). Upon E2 treatment, the exemplary MOTERA scores of ESR1-WT bearing PDX lines still remained significantly lower than those of ESR1 mutated tumors, although in several cases expression levels rose above the cutoff established in E2-deprived conditions (FIG. 7B).

An independent RNA-Seq data set of 55 ER+ mRNA positive MBC cases from the MET500 study (9) was used to further evaluate the performance of the exemplary MOTERA gene signature in tumor samples. Signature scores were significantly elevated in tumors expressing ESR1 LBD point mutations, such as Y537S and D538G, versus ESR1-WT samples (FIG. 7C). Two ESR1 fusions that were functionally studied in FIG. 2 (ESR1-e6>ARNT2-e18 and ESR1-e6>ARID1B) were both originally identified from the MET500 study. As expected, the ESR1-e6>ARNT2-e18 fusion drove a high exemplary MOTERA score in the sample in which it was identified. Against predictions, the functionally inactive ESR1-e6>ARID1B fusion also had a positive exemplary MOTERA signature score. However, this patient sample also harbored an ESR1-D538G LBD mutation, likely explaining the discordance. In terms of performance, the exemplary MOTERA signature score significantly distinguished active ESR1 mutations (Y537S, D538G, and Y537C point mutations and the ESR1-e6>ARNT2-e18 fusion) from WT ESR1, with a sensitivity of 92.9% and a specificity of 78.0% for an AUC of 88.7% (95% confidence interval, 80.0%-97.3%; FIG. 7D).

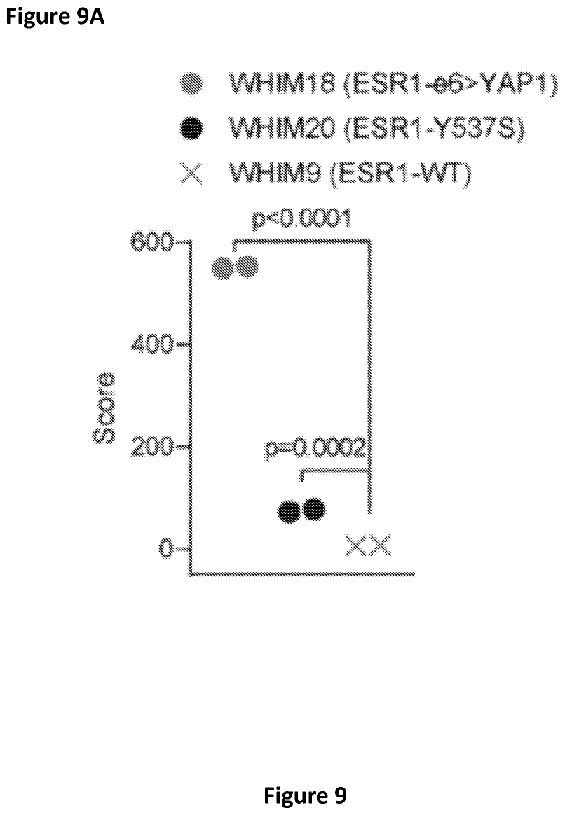

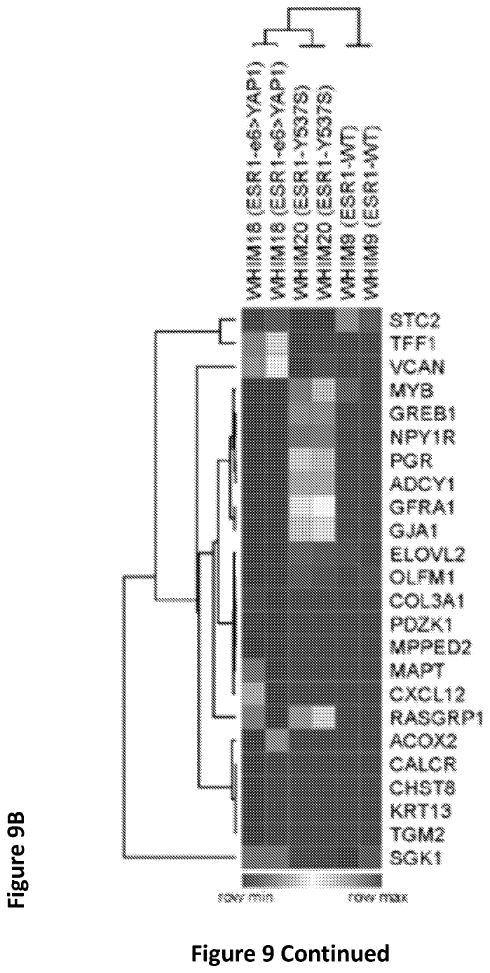

The efficacy of assays comprising labeled probe-based hybridization analysis (e.g., comprising NanoString probes) (e.g., probes targeting SEQ ID NO: 1 to SEQ ID NO: 28) in accurately predicting higher MOTERA scores was validated in patient-derived xenograft (PDX) tumors grown in immune-deficient mice that either expressed an active ESR1 fusion protein (e.g., WHIM18; ESR1-YAP1 fusion) or ESR1 LBD point mutation (e.g., WHIM20; ESR1-Y537S) compared to WHIM9 expressing wild-type (WT) ESR1. The inventors performed this testing from freshly isolated tumors. The assays comprising labeled probe-based hybridization analysis probes (e.g., NanoString probes) successfully detected significantly higher MOTERA scores in PDXs expressing an active ESR1 fusion protein (WHIM18) or ESR1 LBD point mutant (WHIM20) when compared to the WT ESR1 expressing WHIM9 PDX (FIGS. 9A and 9B).

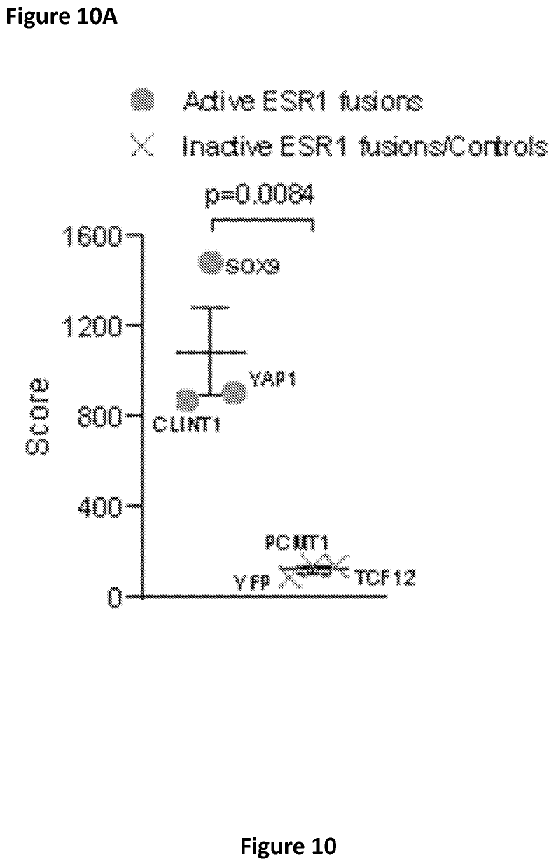

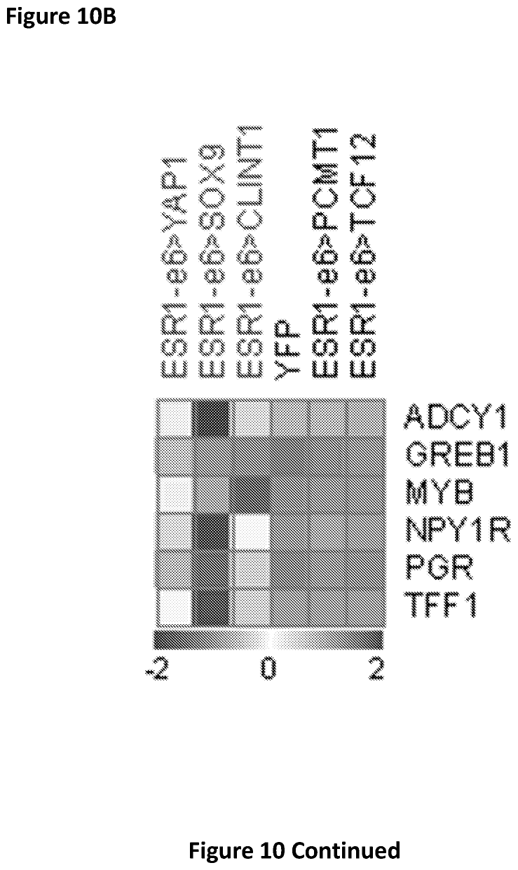

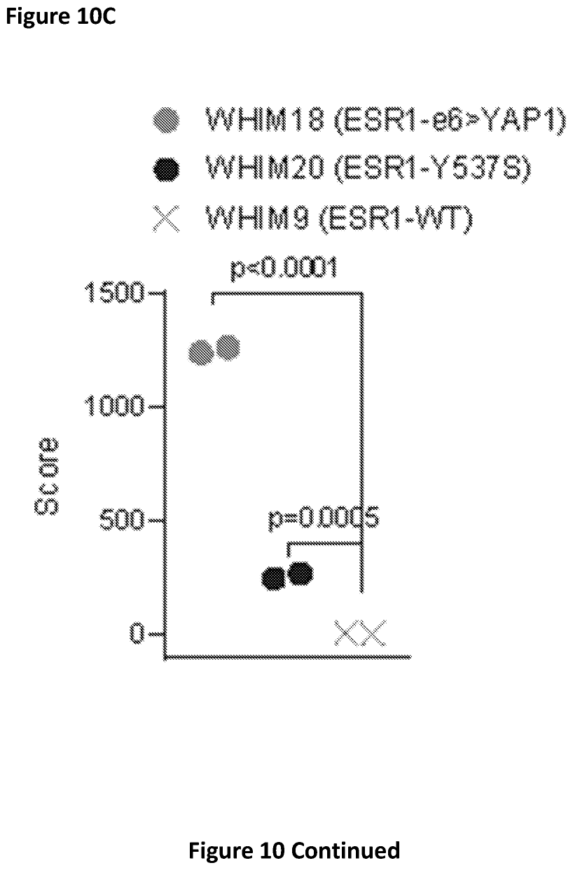

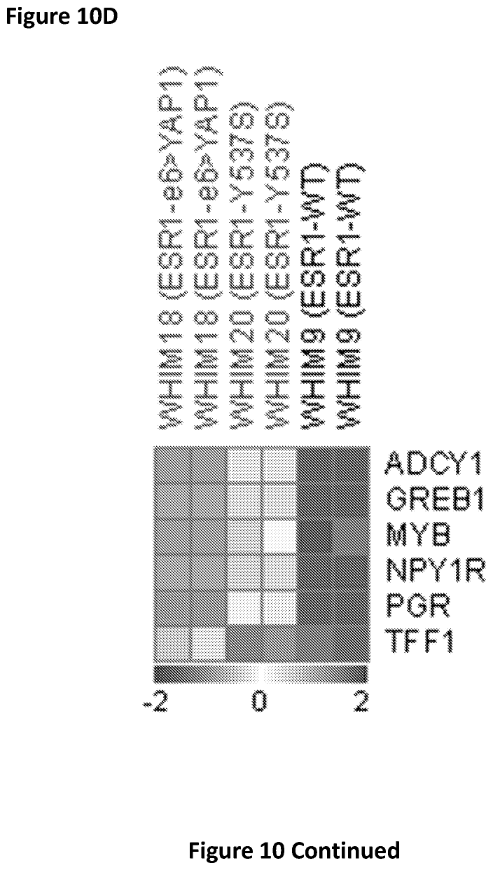

Efficacy of ESR1 activity discriminatory power of a MOTERA signature of 6 genes (ADCY1, GREB1, MYB, NPY1R, PGR, and TFF1) was determined. Results showed that a 6 gene panel provided ESR1 activity discriminatory power (FIG. 10A to 10D) in both cell line and PDX models. The 6 gene signature successfully predicted the presence of active ESR1 fusions/mutant proteins in T47D cells and PDX tumors, indicated by significantly higher MOTERA scores.

Assays comprising labeled probe-based hybridization analysis (e.g., NanoString probes) are utilized to predict MOTERA scores in formalin-fixed paraffin-embedded (FFPE) sample PDX tumor models. The efficacy of MOTERA scores to determine fusion ESR1 mutant activity is validated in FFPE sample PDX models.

Alternative internal control genes and/or number of internal control genes can be used, and the number of MOTERA signature genes can be reduced to a subset (e.g., 6, 10, 14, 18, or 22, or any range derivable therein) of the MOTERA genes described herein.

### Example 7—Discussion and Significance

The data presented herein clearly demonstrate that most in-frame ESR1-e6 fusion proteins derived from inter-chromosomal translocations are drivers of ET resistance. Hitherto the clinical importance of ESR1 gene translocation has been underappreciated because the diversity of C-terminal partner genes creates a considerable diagnostic challenge. Fluorescence In Situ Hybridization (FISH) and PCR approaches that require the identification of both partners in a gene fusion event are not applicable. Even in the case of ESR1-e6>ARNT2, where we identified two examples, the ARNT2 exons present at the fusion junctions were different. Break-apart FISH probes can be considered in a setting where only one partner in the fusion is known. However, this approach does not identify the unknown 3′ partner gene or the reading frame, which is critical because inactive out-of-frame fusions are common (Table 2). RNA-Seq is clearly an applicable unbiased discovery approach, but sensitive detection requires the identification of a sufficient number of fusion junction reads to confidently diagnose the presence of an in-frame translocation. When RNA-Seq coverage is low, or the RNA is of low quality, fusion junction sequences could easily remain undetected.

TABLE 2
Summary of ESR1 gene fusions in ER+ breast cancer

| ESR1 | | | | |
|---|---|---|---|---|
| fusions | Frame | Examples | Source | Mechanisms |
| Active | In-frame | ESR1−e6 > YAP1 (4) | Metastatic | Generated by inter-chromosomal |
| fusions | | ESR1−e6 > PCDH11X (5) | tumors | translocation |
| | | ESR1−e6 > SOX9 (6) | | Produce stable ERα fusion protein |
| | | ESR1−e6 > ARNT2−e18* | | Upregulate transcriptional activation of ERα |
| | | ESR1−e6 > ARNT2−e2 (7) | | target and EMT genes |
| | | ESR1−e6 > LPP (7) | | Drive ET-resistant tumor growth and |
| | | ESR1−e6 > NCOA1 (7) | | metastasis |
| | | ESR1−e6 > CLINT1 (7) | | |
| | | ESR1−e6 > GRIP1 (7) | | |
| | | ESR1−e6 > TNRC6B (7) | | |
| | 5′ UTR- | ESR1−e2 > CCDC170 (8) | Primary | Generated by tandem duplication |
| | CDS | ESR1−e2 > C6orf211 (9) | tumors | Produce truncated partner protein (rather |
| | | | | than a chimeric protein) |
| | | | | Reduce ET sensitivity |
| Cell | In-frame | ESR1−e6 > DAB2 (6) | Metastatic | Generated by inter-chromosomal |
| context- | | | tumors | translocation |
| dependent | | | | Produce stable ERα fusion protein |
| fusion | | | | Drive hormone-independent growth in |
| | | | | MCF7 but not T47D cells |
| Inactive | In-frame | ESR1−e6 > GYG1 (6) | Metastatic | Generated by inter-, or intra-chromosomal |
| fusions | | ESR1−e6 > PCMT1* | tumors | translocation |
| | | ESR1−e6 > ARID1B* | | Produce stable ERα fusion protein |
| | | ESR1−e6 > TCF12 (7) | | Do not drive E2-independent growth |
| | | ESR1−e6 > NOP2 (5) | Primary | Generated by inter-chromosomal |
| | | ESR1−e7 > POLH (5) | tumors | translocation or tandem duplication |
| | | ESR1−e6 > AKAP12 (5, 9) | | |
| | Out-of- | ESR1−e3 > CCDC170 (5) | | Do not drive E2-independent growth |
| | frame | ESR1−e4 > CCDC170 (5) | | |
| | | ESR1−e5 > CCDC170 (5) | | |
| | | ESR1−e6 > AKR1D1 (5) | | |

*Personal communication, Dr. Dan Robinson

Adding to the difficulty of understanding the clinical significance of ESR1 gene fusions is the fact that only a subset of ESR1 fusion proteins are active, and therefore clinically actionable. Consistent rules to diagnose whether a fusion is active based on the known functions of the C-terminal fusion partners proved hard to define. While ESR1-e6 fusions with YAP1, SOX9, ARNT2, LPP, and NCOA1 are all known positive regulators of transcription and produce active fusion proteins, our analysis of the ESR1-e6>TCF12 fusion protein produced an interesting exception. TCF12 encodes a bHLH E-box TF and its two TADs (29) are present in the fusion. Nonetheless the synthetic ESR1-e6>TCF12 cDNA was inactive in both T47D and MCF7 cells. We cannot exclude the possibility that this particular fusion is only active in the context of the cancer in which it evolved, i.e. the indicator cell lines we used lack the requisite coactivators. If truly inactive, however, the ESR1-e6>TCF12 fusion event raises the question of how this example could have been selected during clonal evolution. A potential explanation is provided by the ESR1-e6>ARID1B fusion protein, which is transcriptionally inactive with a 3′ partner gene related to the established tumor suppressor ARID1A (30,31). It has been proposed that TCF12 encodes a tumor suppressor (29,32). Thus, selection of transcriptionally inactive ESR1 fusions could be explained if these fusions inactivate tumor suppressor functions encoded by the 3′ partner gene. One could speculate that these putative ESR1 tumor suppressor fusion proteins act in a dominant negative fashion, thereby interrupting the function of the remaining intact TCF12 or the ARID1A activity. Multiple active non-TF/CoA fusions (PCDH11X, DAB2, CLINT1, GRIP1 and TNRC6B) dramatically add to the complex landscape of ESR1 fusion genes. The activity of these fusions cannot, by definition, be predicted from an understanding of the normal function of each 3′ partner gene involved since none are known to be a TF or CoA and the wild-type protein is not nuclear-localized. Presumably the fusion partners have diverse protein-protein interaction domains that are subverted for the purposes of activating gene transcription in the context of a pathological fusion with ESR1. These questions can be addressed in follow up mechanistic studies.

One diagnostic approach after the detection of an in-frame ESR1 fusion gene would be to test the newly identified example in vitro. However, this is generally considered inefficient for clinical care, and may not always produce an accurate result. These concerns stimulated the development of MOTERA gene signatures to screen for tumors driven by the diverse somatic events that activate ESR1 through the presence of a diagnostic gene signature. In a setting where an ESR1 activating mutation has already been identified, a MOTERA signature could be used to confirm the mutant ESR1 gene is indeed driving ET-resistance. However, a MOTERA signature is likely to be of significant value in the setting where a canonical ESR1 LBD point mutation has not been detected. Here, a high MOTERA score would warrant further investigation to detect a functional in-frame ESR1-e6 fusion, and can influence clinical options. Reflex diagnostic approaches for these cases could include unbiased RNA-Seq, ESR1-specific 3′ Rapid Amplification of cDNA Ends (3′-RACE) or break-apart ESR1 FISH. While break-apart FISH would not identify the C-terminal partner, its presence has already been signaled by a positive MOTERA score implying the unknown partner in the chimera is transcriptionally active.

Analysis of the MET500 data indicates that MBC with high exemplary MOTERA scores but without an ESR1 point mutation detected by genome sequencing or translocation detected by RNA-Seq are not infrequent (FIG. 7C). Possibilities for these cases include: 1) the RNA-Seq result was false-negative for the presence of an active ESR1 fusion; 2) the exome sequencing was a false-negative for the presence of an ESR1 mutation; 3) the MOTERA score was a false-positive that reflects wild-type ERα activity because the sample was taken when the patient was not taking ET and the tumor was still E2-dependent; and 4) some wild-type ERα MBC persist by expressing a similar transcriptional signature that might be driven by other mechanisms, like transcription factors other than ERα.

An important focus for future studies will be to determine the clinical characteristics of ESR1 fusion-driven tumors. Of particular interest is an examination of the metastatic spread associated with tumors expressing ESR1 gene fusions, as in mouse xenograft systems active ESR1 fusions drive lung metastasis (7). Distinct from WT ERα, active ESR1 fusions strongly induce EMT-related genes, which we functionally annotated using motility and invasion assays. This property differentiates MOTERA signatures from other gene sets that measure activity of the ERα pathway, such as the Hallmark early/late estrogen response gene set. Consistent with this, the exemplary MOTERA scores of ESR1 mutated PDX tumors were still significantly higher than those of ESR1-WT bearing lines that received E2 treatment. Interestingly, EMT-related gene expression is elevated during mammary gland development as the nascent ducts invade the mammary fat pad, and then EMT gene expression is reduced after puberty (33,34). Thus, active ESR1-e6 fusion proteins may be reactivating a developmental EMT program that is usually silenced in mature breast epithelial cells. Specific examples of ESR1 fusion-induced genes in the exemplary MOTERA signature that are related to metastasis include SGK1, which encodes serum- and glucocorticoid-inducible kinase 1 and promotes breast cancer bone metastasis (35). VCAN encodes versican, whose expression is significantly correlated with metastasis and poor overall survival (36). GJA1 encodes connexin-43, a gap junction protein that mediates tumor cell migration and invasion (37,38). GFRA1 encodes GFRα that acts as a co-receptor in conjunction with the RET receptor, and activation of GFRα-RET signaling by binding the glial derived neurotrophic factor (GDNF) ligand leads to ERα serine phosphorylation and enhanced transcriptional activity (39).

At least one ESR1 fusion partner gene described herein has been observed in other settings. Gene fusions involving LPP, the gene encoding the Lipoma Preferred Partner protein, such as a recurrent HMGA2-LPP fusion have been found in multiple tumors, including lipoma (40), pulmonary chondroid hamartomas (41), and chondromas (42). In leukemia, an MLL-LPP fusion has been identified (43). Similar to the ESR1-e6>LPP fusion, these fusions preserve the three C-terminal LIM domains encoded by the LPP gene, which serve as the binding site for the ETS domain transcription factor PEA3 and contain coactivator activity (44). It is therefore likely that in larger studies, some ESR1 gene fusions will be observed to be recurrent, making the diagnosis of some ESR1 translocations easier.

ESR1-e6 gene fusions are part of the spectrum of the somatic mutations that constitutively activate ESR1 proteins in advanced ER+ breast cancer to drive poor outcomes. A MOTERA signature can help answer the question of how common these events are, because it will focus sensitive fusion detection approaches on cases where there is transcriptional evidence for an activating ESR1 fusion (or mutation) that has not been diagnosed yet. As the clinical significance of ESR1 gene fusions becomes more widely recognized and the diagnostic approach becomes more efficient, specific treatment approaches for tumors expressing active ESR1 fusion proteins can be developed.

### REFERENCES

1. Osborne C K, Schiff R. Mechanisms of endocrine resistance in breast cancer. Annu Rev Med 2011; 62:233-47
2. Ma C X, Reinert T, Chmielewska I, Ellis M J. Mechanisms of aromatase inhibitor resistance. Nat Rev Cancer 2015; 15:261-75
3. Dustin D, Gu G, Fuqua S A W. ESR1 mutations in breast cancer. Cancer 2019; 125:3714-28
4. Fribbens C, O'Leary B, Kilburn L, Hrebien S, Garcia-Murillas I, Beaney M, et al. Plasma ESR1 Mutations and the Treatment of Estrogen Receptor-Positive Advanced Breast Cancer. J Clin Oncol 2016; 34:2961-8
5. Spoerke J M, Gendreau S, Walter K, Qiu J, Wilson T R, Savage H, et al. Heterogeneity and clinical significance of ESR1 mutations in ER-positive metastatic breast cancer patients receiving fulvestrant. Nat Commun 2016; 7:11579
6. Li S, Shen D, Shao J, Crowder R, Liu W, Prat A, et al. Endocrine-therapy-resistant ESR1 variants revealed by genomic characterization of breast-cancer-derived xenografts. Cell Rep 2013; 4:1116-30
7. Lei J T, Shao J, Zhang J, Iglesia M, Chan D W, Cao J, et al. Functional Annotation of ESR1 Gene Fusions in Estrogen Receptor-Positive Breast Cancer. Cell Rep 2018; 24:1434-44 e7
8. Hartmaier R J, Trabucco S E, Priedigkeit N, Chung J H, Parachoniak C A, Vanden Borre P, et al. Recurrent hyperactive ESR1 fusion proteins in endocrine therapy-resistant breast cancer. Ann Oncol 2018; 29:872-80
9. Robinson D R, Wu Y M, Lonigro R J, Vats P, Cobain E, Everett J, et al. Integrative clinical genomics of metastatic cancer. Nature 2017; 548:297-303
10. Duncan J S, Whittle M C, Nakamura K, Abell A N, Midland A A, Zawistowski J S, et al. Dynamic reprogramming of the kinome in response to targeted MEK inhibition in triple-negative breast cancer. Cell 2012; 149:307-21
11. Li B, Dewey C N. RSEM: accurate transcript quantification from RNA-Seq data with or without a reference genome. BMC Bioinformatics 2011; 12:323
12. Langmead B, Salzberg S L. Fast gapped-read alignment with Bowtie 2. Nat Methods 2012; 9:357-9
13. Zhang X, Claerhout S, Prat A, Dobrolecki L E, Petrovic I, Lai Q, et al. A renewable tissue resource of phenotypically stable, biologically and ethnically diverse, patient-derived human breast cancer xenograft models. Cancer Res 2013; 73:4885-97
14. DeRose Y S, Wang G, Lin Y C, Bernard P S, Buys S S, Ebbert M T, et al. Tumor grafts derived from women with breast cancer authentically reflect tumor pathology, growth, metastasis and disease outcomes. Nat Med 2011; 17:1514-20
15. Robin X, Turck N, Hainard A, Tiberti N, Lisacek F, Sanchez J C, et al. pROC: an open-source package for R and S+ to analyze and compare ROC curves. BMC Bioinformatics 2011; 12:77
16. Foulds C E, Feng Q, Ding C, Bailey S, Hunsaker T L, Malovannaya A, et al. Proteomic analysis of coregulators bound to ERalpha on DNA and nucleosomes reveals coregulator dynamics. Mol Cell 2013; 51:185-99
17. Gates L A, Gu G, Chen Y, Rohira A D, Lei J T, Hamilton R A, et al. Proteomic profiling identifies key coactivators utilized by mutant ERalpha proteins as potential new therapeutic targets. Oncogene 2018; 37:4581-98
18. Bagchi M K, Tsai S Y, Tsai M J, O'Malley B W. Identification of a functional intermediate in receptor activation in progesterone-dependent cell-free transcription. Nature 1990; 345:547-50
19. UniProt C. UniProt: the universal protein knowledgebase in 2021. Nucleic Acids Res 2021; 49: D480-D9
20. LaRese T P, Rheaume B A, Abraham R, Eipper B A, Mains R E. Sex-Specific Gene Expression in the Mouse Nucleus Accumbens Before and After Cocaine Exposure. J Endocr Soc 2019; 3:468-87
21. Howell A, Osborne C K, Morris C, Wakeling A E. ICI 182,780 (Faslodex): development of a novel, “pure” antiestrogen. Cancer 2000; 89:817-25
22. Carroll J S, Brown M. Estrogen receptor target gene: an evolving concept. Mol Endocrinol 2006; 20:1707-14
23. Ye X, Tam W L, Shibue T, Kaygusuz Y, Reinhardt F, Ng Eaton E, et al. Distinct EMT programs control normal mammary stem cells and tumour-initiating cells. Nature 2015; 525:256-60
24. Cano A, Perez-Moreno M A, Rodrigo I, Locascio A, Blanco M J, del Barrio M G, et al. The transcription factor snail controls epithelial-mesenchymal transitions by repressing E-cadherin expression. Nat Cell Biol 2000; 2:76-83
25. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA 2005; 102:15545-50
26. Liberzon A, Subramanian A, Pinchback R, Thorvaldsdottir H, Tamayo P, Mesirov J P. Molecular signatures database (MSigDB) 3.0. Bioinformatics 2011; 27:1739-40
27. Priestley P, Baber J, Lolkema M P, Steeghs N, de Bruijn E, Shale C, et al. Pan-cancer whole-genome analyses of metastatic solid tumours. Nature 2019; 575:210-6
28. Sikora M J, Cooper K L, Bahreini A, Luthra S, Wang G, Chandran U R, et al. Invasive lobular carcinoma cell lines are characterized by unique estrogen-mediated gene expression patterns and altered tamoxifen response. Cancer Res 2014; 74:1463-74
29. Labreche K, Simeonova I, Kamoun A, Gleize V, Chubb D, Letouze E, et al. TCF12 is mutated in anaplastic oligodendroglioma. Nat Commun 2015; 6:7207
30. Nagl N G, Jr., Patsialou A, Haines D S, Dallas P B, Beck G R, Jr., Moran E. The p270 (ARID1A/SMARCF1) subunit of mammalian SWI/SNF-related complexes is essential for normal cell cycle arrest. Cancer Res 2005; 65:9236-44
31. Shain A H, Giacomini C P, Matsukuma K, Karikari C A, Bashyam M D, Hidalgo M, et al. Convergent structural alterations define SWItch/Sucrose NonFermentable (SWI/SNF) chromatin remodeler as a central tumor suppressive complex in pancreatic cancer. Proc Natl Acad Sci USA 2012; 109: E252-9
32. Chen Q B, Liang Y K, Zhang Y Q, Jiang M Y, Han Z D, Liang Y X, et al. Decreased expression of TCF12 contributes to progression and predicts biochemical recurrence in patients with prostate cancer. Tumour Biol 2017; 39:1010428317703924
33. Sikandar S S, Kuo A H, Kalisky T, Cai S, Zabala M, Hsieh R W, et al. Role of epithelial to mesenchymal transition associated genes in mammary gland regeneration and breast tumorigenesis. Nat Commun 2017; 8:1669
34. Foubert E, De Craene B, Berx G. Key signalling nodes in mammary gland development and cancer. The Snaill-Twistl conspiracy in malignant breast cancer progression. Breast Cancer Res 2010; 12:206
35. Zhang Z, Xu Q, Song C, Mi B, Zhang H, Kang H, et al. Serum- and Glucocorticoid-inducible Kinase 1 is Essential for Osteoclastogenesis and Promotes Breast Cancer Bone Metastasis. Mol Cancer Ther 2020; 19:650-60
36. Mitsui Y, Shiina H, Kato T, Maekawa S, Hashimoto Y, Shiina M, et al. Versican Promotes Tumor Progression, Metastasis and Predicts Poor Prognosis in Renal Carcinoma. Mol Cancer Res 2017; 15:884-95
37. Pollmann M A, Shao Q, Laird D W, Sandig M. Connexin 43 mediated gap junctional communication enhances breast tumor cell diapedesis in culture. Breast Cancer Res 2005; 7: R522-34
38. Kotini M, Mayor R. Connexins in migration during development and cancer. Dev Biol 2015; 401:143-51
39. Plaza-Menacho I, Morandi A, Robertson D, Pancholi S, Drury S, Dowsett M, et al. Targeting the receptor tyrosine kinase RET sensitizes breast cancer cells to tamoxifen treatment and reveals a role for RET in endocrine resistance. Oncogene 2010; 29:4648-57
40. Crombez K R, Vanoirbeek E M, Van de Ven W J, Petit M M. Transactivation functions of the tumor-specific HMGA2/LPP fusion protein are augmented by wild-type HMGA2. Mol Cancer Res 2005; 3:63-70
41. Rogalla P, Lemke I, Kazmierczak B, Bullerdiek J. An identical HMGIC-LPP fusion transcript is consistently expressed in pulmonary chondroid hamartomas with t(3;12)(q27-28;ql4-15). Genes Chromosomes Cancer 2000; 29:363-6
42. Dahlen A, Mertens F, Rydholm A, Brosjo O, Wejde J, Mandahl N, et al. Fusion, disruption, and expression of HMGA2 in bone and soft tissue chondromas. Mod Pathol 2003; 16:1132-40
43. Daheron L, Veinstein A, Brizard F, Drabkin H, Lacotte L, Guilhot F, et al. Human LPP gene is fused to MLL in a secondary acute leukemia with a t(3;11) (q28;q23). Genes Chromosomes Cancer 2001; 31:382-9
44. Guo B, Sallis R E, Greenall A, Petit M M, Jansen E, Young L, et al. The LIM domain protein LPP is a coactivator for the ETS domain transcription factor PEA3. Mol Cell Biol 2006; 26:4529-38

1-58. (canceled)

59. A method of treating metastatic breast cancer comprising,
administering an effective amount of a non-ET resistant therapeutic regimen to a patient determined to have a biological sample with increased tumor cell expression of at least six genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

60-63. (canceled)

64. The method of claim 59, wherein the patient is determined to have a biological sample with increased tumor cell expression of genes: ADCY1, GREB1, MYB, NPY1R, PGR, and TFF1.

65. (canceled)

66. The method of claim 59, wherein the method further comprises measuring expression levels of one or more internal controls, wherein the one or more internal controls comprise B2M, GAPDH, PSMC4, and/or PUM1.

67. (canceled)

68. The method of claim 59, wherein the level of gene activity is increased relative to a control and is identified using a nucleotide quantification assay.

69. (canceled)

70. The method of claim 68, wherein the nucleotide quantification assay comprises a labeled probe-based hybridization analysis assay or RNA sequencing.

71. The method of claim 70, wherein the assay comprises one or more targeting probe/primers which comprises, or comprises a sequence complementary to, any one of SEQ ID NO: 1 to SEQ ID NO: 28.

72. The method of claim 70, wherein the labeled probe-based hybridization analysis assay comprises a NanoString assay.

73. The method of claim 59, wherein the cancer is ERα+ metastatic breast cancer (MBC).

74. The method of claim 59, wherein the non-ET resistant therapeutic regimen comprises a CDK4/6 inhibitor.

75. The method of claim 74, wherein the non-ET resistant therapeutic regimen further comprises one or more of a SERM, an aromatase inhibitor, and/or a SERD.

76. The method of claim 74, wherein the CDK4/6 inhibitor is abemaciclib, palbociclib, or ribociclib.

77. (canceled)

78. The method of claim 59, wherein a reflexive diagnostic test is performed following tumor cell gene expression determination and prior to treatment regimen initiation.

79. The method of claim 78, wherein the reflexive diagnostic test is selected from unbiased RNA-Seq, whole exome sequencing, ESR1-specific 3′ Rapid Amplification of cDNA ends (3′-RACE), and break-apart ESR1 fluorescence in situ hybridization (FISH).

80-83. (canceled)

84. The method of claim 59, wherein the administering of the non-ET resistant therapeutic regimen occurs within 1 month after tumor cell gene expression determination.

85. The method of claim 59, wherein the biological sample is a primary tumor tissue sample or a metastatic tumor lesion sample.

86-115. (canceled)

116. A method for treating cancer in a patient, the method comprising administering a cancer therapy to the patient after determining whether the patient has a wild-type, mutant or translocated estrogen receptor alpha (ERα) protein by
measuring a biological sample from the patient for estrogen response gene expression and/or epithelial to mesenchymal transition (EMT) gene expression, wherein the cancer therapy comprises an effective amount of a CDK 4/6 inhibitor when the patient has a mutant or translocated estrogen receptor alpha ERα protein and wherein the cancer therapy comprises an effective amount of an Endocrine Therapy (ET) and a CDK 4/6 inhibitor when the patient has a wild-type ERα protein.

117-120. (canceled)

121. The method of claim 116, wherein determination of estrogen response gene expression comprises measuring expression levels of genes: ADCY1, GREB1, MYB, NPY1R, PGR, and TFF1.

122-163. (canceled)

164. The method of claim 116, wherein the CDK4/6 inhibitor is abemaciclib, palbociclib, or ribociclib and/or wherein the ET comprises one or more of a SERM, an aromatase inhibitor, and/or a SERD.

165-186. (canceled)

187. A kit comprising oligonucleotides capable of hybridizing to, and facilitating expression level determination of, at least six genes selected from: ACOX2, ADCY1, ADRA2A, AFF3, AMZ1, BFSP2, BMPR1B, C14orf182, CALCR, CCDC88A, CD109, CD34, CHST8, COL3A1, CT62, CXCL12, DOK7, DSCAML1, ELOVL2, FLT4, FMN1, GATA4, GFRA1, GJA1, GREB1, GREM2, HEY2, IFITM10, IGF2, KCNH1, KRT13, MAPT, MDGA1, MPPED2, MYB, NKAIN1, NPY1R, NXPH3, OLFM1, PDZK1, PGLYRP2, PGR, PPP2R2C, PRSS56, RASGRP1, RBM24, RIMS4, ROBO3, SEMA3A, SERPINA6, SGK1, SLC47A1, SOX5, SPINK13, SPINK4, SPINK5, STC1, STC2, SUSD3, SYTL5, TFF1, TGM2, UGT3A2, VCAN, WT1, and ZNF385B.

188-191. (canceled)

192. The kit of claim 187, comprising oligonucleotides capable of hybridizing to, and facilitating expression level determination of six genes: ADCY1, GREB1, MYB, NPY1R, PGR, and TFF1.

193-246. (canceled)
